(19) United States
(12) Patent Application Publication
(10) Pub. No.: US 2007/0244103 A1
(43) Pub. Date: Oct. 18, 2007

(54) Novel Compounds Having an Anti-Bacterial Activity

(75) Inventors: Sabine Pierau, Freiburg (DE); Glenn Dale, Basel (CH); Michael W. Cappi, Munchen (DE); Cornelia Zumbrunn, Basel (CH); Christian Hubschwerlen, Durmenach (FR); Jean P. Surivet, Saint-Louis (FR)

Correspondence Address: EDWARDS ANGELL PALMER & DODGE LLP, P.O. BOX 55874, BOSTON, MA, 02205, US

(73) Assignee: Morphochem Aktiengesellschaft Fur Kombinatorische Chemie, Munchen (DE)

(21) Appl. No.: 11/660,894

(22) PCT Filed: Aug. 25, 2005
(86) PCT No.: PCT/EP05/09204

§ 371 Date: Jun. 20, 2007

(30) Foreign Application Priority Data
Aug. 25, 2004 (DE) 10 2004 041 163.8

(51) Int. Cl. A61K 31/445; A61P 31/04; C07D 401/14; C07D 405/14; C07D 417/14; C07D 419/14

(52) U.S. Cl. 514/227.8; 514/230.5; 514/233.5; 514/249; 514/300; 514/311; 544/105; 544/127; 544/353; 544/47; 546/123; 546/152

(57) ABSTRACT

The present invention describes novel anti-bacterial compounds of formula (I).
Q-A-R³ (I) These compounds are, amongst others, of interest as inhibitors of DNA gyrase.

Resistance to the antibiotics used currently has increased appreciably in many countries of the world in recent years and in some cases has assumed alarming proportions. The main problem is that those pathogens exhibit not just a single resistance but, as a rule, multiple resistances. This is true especially of some gram-positive pathogen groups, such as staphylococci, pneumococci and enterococci (S. Ewig et al., Antibiotika-Resistenz bei Erregern ambulant erworbener Atemwegsinfektionen (Antibiotic resistance in pathogens of outpatient-acquired respiratory tract infections), Chemother. J. 2002, 11, 12-26; F. Tenover, Development and spread of bacterial resistance to antimicrobial agents: an overview, Clin. Infect. Dis. 2001 Sep. 15, 33 Suppl. 3, 108-115).

A long-feared development has recently occurred: In the USA, the first strain of Staphylococcus aureus has been described that is not only resistant to methicillin but also highly resistant to vancomycin (Centers for Disease Control and Prevention, Staphylococcus aureus resistant to vancomycin—United States, 2002, MMWR 2002, 51, 565-567). In addition to hygiene measures in hospitals, therefore, increased efforts are also required to find new antibiotics that as far as possible have a novel structure and a novel mechanism of action so as to be effective against those problem bacteria.

The present invention describes new kinds of compounds having anti-bacterial activity. These compounds are, amongst others, of interest as inhibitors of DNA gyrase.

The present invention relates to compounds of the general formula (I):
Q-A-R³ (I)
wherein
Q is a group having the following structure: 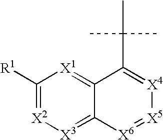
R¹ is a hydrogen atom, a halogen atom, a hydroxy, an amino, a mercapto, an alkyl, a heteroalkyl, an alkyloxy, a heteroalkyloxy, a cycloalkyl, a heterocycloalkyl, an alkylcycloalkyl, a heteroalkylcycloalkyl, a cycloalkyloxy, an alkylcycloalkyloxy, a heterocycloalkyloxy or a heteroalkylcycloalkyloxy group,
X¹, X², X³, X⁴, X⁵ and X⁶ are each independently of the others nitrogen atoms or groups of formula CR²,
R² is a hydrogen atom, a halogen atom, or a hydroxy, amino, alkyl, alkenyl, alkynyl or heteroalkyl group,
R³ is selected from the following groups: 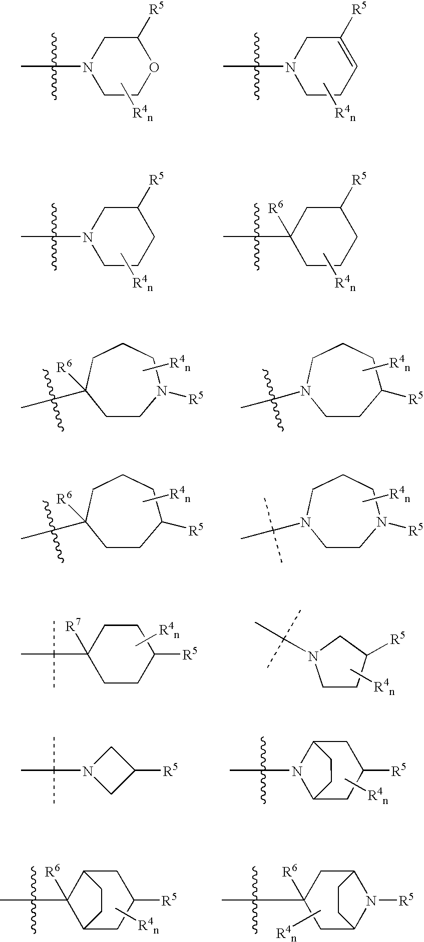
the radicals R⁴, each independently of any other(s), are a halogen atom, a hydroxy, an amino, a nitro or a mercapto group, an alkyl, an alkenyl, an alkynyl, a heteroalkyl, an aryl, a heteroaryl, a cycloalkyl, an alkylcycloalkyl, a heteroalkylcycloalkyl, a heterocycloalkyl, an aralkyl or a heteroaralkyl radical, or two of the radicals R⁴ together form an aryl, heteroaryl, cycloalkyl, heterocycloalkyl, alkylcycloalkyl, heteroalkylcycloalkyl, aralkyl or a heteroaralkyl ring system,
R⁵ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radical,
R⁶ is a hydrogen atom or R⁷,
R⁷ is a halogen atom, or a hydroxy, alkyl, alkenyl, alkynyl or heteroalkyl group,
n is 0, 1 or 2,
A is selected from the following groups: —NR⁸CO—, —CR⁹R¹⁰CO—, —CR⁹R¹⁰SO₂—, —NR⁸SO₂—, —CR⁹R¹⁰CR¹¹(OR¹²)—, —CONR⁸—, —CR⁹R¹⁰NR⁸—, —CR⁹R¹⁰O—, —CR⁹R¹⁰S—, —CR¹¹(OR¹²)CR¹³R¹⁴—, —COCR¹³R¹⁴— and —CR⁹R¹⁰CR¹³R¹⁴—,
R⁸ is a hydrogen atom, a trifluoromethyl, a (C₁₋₆)alkyl, a (C₂₋₆)alkenyl, a (C₁₋₆)alkoxycarbonyl, a (C₁₋₆)alkylcarbonyl or an aminocarbonyl group wherein the amino group, if applicable, may be substituted by a (C₁₋₆)alkoxycarbonyl, a (C₁₋₆)alkylcarbonyl, a (C₂₋₆)alkenyloxycarbonyl, a (C₂₋₆)alkenylcarbonyl, a (C₁₋₆)alkyl, a (C₂₋₆)alkenyl and, if applicable, substituted further on by a (C₁₋₆)alkyl or a (C₂₋₆)alkenyl group,
the radicals R⁹, R¹⁰, R¹¹, R¹³ and R¹⁴ are each independently of the others a hydrogen atom, a halogen atom, an azide, a trifluoromethyl, a hydroxy, an amino, a (C₁₋₆)alkyloxy, a (C₁₋₆)alkylthio, a (C₁₋₆)alkyl, a (C₂₋₆)alkenyl, a (C₁₋₆)alkoxycarbonyl, a (C₂₋₆)alkenyloxycarbonyl, a (C₁₋₆)alkylsulphonyl, a (C₂₋₆)alkenylsulphonyl or a (C₁₋₆)amino-sulphonyl group wherein the amino group may be, if applicable, substituted by a (C₁₋₆)alkyl or a phenyl group,
R¹² is a hydrogen atom, a trifluoromethyl, a (C₁₋₆)alkyl, a (C₂₋₆)alkenyl, a (C₁₋₆)alkoxycarbonyl, a (C₁₋₆)alkylcarbonyl or an aminocarbonyl group wherein the amino group may be, if applicable, substituted by a (C₁₋₆)alkoxycarbonyl, a (C₁₋₆)alkylcarbonyl, a (C₂₋₆)alkenyloxycarbonyl, a (C₂₋₆)alkenylcarbonyl, a (C₁₋₆)alkyl, a (C₂₋₆)alkenyl group and, if applicable, substituted further on by a (C₁₋₆)alkyl or a (C₂₋₆)alkenyl group,
or a pharmacologically acceptable salt, solvate, hydrate or a pharmacologically acceptable formulation thereof.

The expression alkyl refers to a saturated, straight-chain or branched hydrocarbon group that contains from 1 to 20 carbon atoms, preferably from 1 to 12 carbon atoms, especially from 1 to 6 carbon atoms, for example a methyl, ethyl, propyl, iso-propyl, n-butyl, iso-butyl, tert-butyl, n-pentyl, n-hexyl, 2,2-dimethylbutyl or n-octyl group.

The expressions alkenyl and alkynyl refer to at least partially unsaturated, straight-chain or branched hydrocarbon groups that contain from 2 to 20 carbon atoms, preferably from 2 to 12 carbon atoms, especially from 2 to 6 carbon atoms, for example an ethenyl, allyl, acetylenyl, propargyl, isoprenyl or hex-2-enyl group. Preferably, alkenyl groups have one or two (especially one) double bond(s) and alkynyl groups have one or two (especially one) triple bond(s).

Furthermore, the terms alkyl, alkenyl and alkynyl can refer to groups in which one or more hydrogen atoms have been replaced each independently of the others by a halogen atom (preferably F or Cl) such as, for example, a 2,2,2-trichloroethyl or a trifluoromethyl group.

The expression heteroalkyl refers to an alkyl, alkenyl or alkynyl group (for example heteroalkenyl, heteroalkynyl) in which one or more (preferably 1, 2 or 3) carbon atoms have been replaced each independently of the others by an oxygen, nitrogen, phosphorus, boron, selenium, silicon or sulphur atom (preferably oxygen, sulphur or nitrogen). The expression heteroalkyl furthermore refers to a carboxylic acid or to a group derived from a carboxylic acid such as, for example, acyl, acyl-alkyl, alkoxycarbonyl, acyloxy, acyloxyalkyl, carboxyalkylamide or alkoxycarbonyloxy.

Examples of heteroalkyl groups are groups of formulae R^{a}—O—Y^{a}—, R^{a}—S—Y^{a}—, R^{a}—N(R^{b})—Y^{a}—, R^{a}—CO—Y^{a}—, R^{a}—O—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—CO—O—Y^{a}—, R^{a}—N(R^{b})—C(═NR^{d})—N(R^{c})—Y^{a}—, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}—, R^{a}—S—CO—N(R^{b})—Y^{a}—, R^{a}—N(R^{b})—CO—S—Y^{a}—, R^{a}—S—CO—O—Y^{a}—, R^{a}—O—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, R^{a} being a hydrogen atom, a C₁-C₆alkyl, a C₂-C₆alkenyl or a C₂-C₆alkynyl group; R^{b} being a hydrogen atom, a C₁-C₆alkyl, a C₂-C₆alkenyl or a C₂-C₆alkynyl group; R^{c} being a hydrogen atom, a C₁-C₆alkyl, a C₂-C₆alkenyl or a C₂-C₆alkynyl group; R^{d} being a hydrogen atom, a C₁-C₆alkyl, a C₂-C₆alkenyl or a C₂-C₆alkynyl group and Y^{a} being a bond, a C₁-C₆alkylene, a C₂-C₆alkenylene or a C₂-C₆alkynylene group, each heteroalkyl group containing at least one carbon atom and it being possible for one or more hydrogen atoms to have been replaced by fluorine or chlorine atoms. Specific examples of heteroalkyl groups are methoxy, trifluoromethoxy, ethoxy, n-propyloxy, isopropyloxy, tert-butyloxy, methoxymethyl, ethoxymethyl, methoxyethyl, methylamino, ethylamino, dimethylamino, diethylamino, isopropylethylamino, methylaminomethyl, ethylaminomethyl, diisopropylaminoethyl, enol ether, dimethylaminomethyl, dimethylaminoethyl, acetyl, propionyl, butyryloxy, acetyloxy, methoxycarbonyl, ethoxycarbonyl, N-ethyl-N-methylcarbamoyl and N-methylcarbamoyl. Further examples of heteroalkyl groups are nitrile, isonitrile, cyanate, thiocyanate, isocyanate, isothiocyanate and alkylnitrile groups. An example of a heteroalkylene group is a group of formula —CH₂CH(OH)— or —CONH—.

The expression cycloalkyl refers to a saturated or partially unsaturated (for example a cyclic group having one, two or more double bonds, such as a cycloalkenyl group), cyclic group that contains one or more rings (preferably 1 or 2), containing from 3 to 14 ring carbon atoms, preferably from 3 to 10 (especially 3, 4, 5, 6 or 7) ring carbon atoms. The expression cycloalkyl refers furthermore to groups in which one or more hydrogen atoms have been replaced each independently of the others by fluorine, chlorine, bromine or iodine atoms or by OH, ═O, SH, ═S, NH₂, ═NH or NO₂ groups, thus, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, bicyclo[4.3.0]nonyl, tetralin, cyclopentylcyclohexyl, fluorocyclohexyl or cyclohex-2-enyl group.

The expression heterocycloalkyl refers to a cycloalkyl group as defined above in which one or more (preferably 1, 2 or 3) ring carbon atoms have been replaced each independently of the others by an oxygen, nitrogen, silicon, selenium, phosphorus or sulphur atom (preferably oxygen, sulphur or nitrogen). A heterocycloalkyl group has preferably 1 or 2 ring(s) containing from 3 to 10 (especially 3, 4, 5, 6 or 7) ring atoms. The expression heterocycloalkyl refers furthermore to groups in which one or more hydrogen atoms have been replaced each independently of the others by fluorine, chlorine, bromine or iodine atoms or by OH, ═O, SH, ═S, NH₂, ═NH or NO₂ groups. Examples are a piperidyl, piperazinyl, morpholinyl, urotropinyl, pyrrolidinyl, tetrahydrothiophenyl, tetrahydropyranyl, tetrahydrofuryl or 2-pyrazolinyl group and also lactams, lactones, cyclic imides and cyclic anhydrides.

The expression alkylcycloalkyl refers to groups containing both cycloalkyl and also alkyl, alkenyl or alkynyl groups in accordance with the above definitions, for example alkylcycloalkyl, cycloalkylalkyl, alkylcycloalkenyl, alkenylcycloalkyl and alkynylcycloalkyl groups. An alkylcycloalkyl group preferably contains a cycloalkyl group that contains one or two ring systems having from 3 to 10 (especially 3, 4, 5, 6 or 7) carbon atoms, and one or two alkyl, alkenyl or alkynyl groups having 1 or 2 to 6 carbon atoms.

The expression heteroalkylcycloalkyl refers to alkylcycloalkyl groups as defined above in which one or more (preferably 1, 2 or 3) carbon atoms have been replaced each independently of the others by an oxygen, nitrogen, silicon, selenium, phosphorus or sulphur atom (preferably oxygen, sulphur or nitrogen). A heteroalkylcycloalkyl group preferably contains 1 or 2 ring systems having from 3 to 10 (especially 3, 4, 5, 6 or 7) ring atoms, and one or two alkyl, alkenyl, alkynyl or heteroalkyl groups having from 1 or 2 to 6 carbon atoms. Examples of such groups are alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocycloalkenyl, the cyclic groups being saturated or mono-, di- or tri-unsaturated.

The expression aryl or Ar refers to an aromatic group that contains one or more rings containing from 6 to 14 ring carbon atoms, preferably from 6 to 10 (especially 6) ring carbon atoms. The expression aryl (or Ar) refers furthermore to groups in which one or more hydrogen atoms have been replaced each independently of the others by fluorine, chlorine, bromine or iodine atoms or by OH, SH, NH₂ or NO₂ groups. Examples are a phenyl, naphthyl, biphenyl, 2-fluorophenyl, anilinyl, 3-nitrophenyl or 4-hydroxyphenyl group.

The expression heteroaryl refers to an aromatic group that contains one or more rings containing from 5 to 14 ring atoms, preferably from 5 to 10 (especially 5 or 6) ring atoms, and contains one or more (preferably 1, 2, 3 or 4) oxygen, nitrogen, phosphorus or sulphur ring atoms (preferably O, S or N). The expression heteroaryl refers furthermore to groups in which one or more hydrogen atoms have been replaced each independently of the others by fluorine, chlorine, bromine or iodine atoms or by OH, SH, NH₂ or NO₂ groups. Examples are 4-pyridyl, 2-imidazolyl, 3-phenylpyrrolyl, thiazolyl, oxazolyl, triazolyl, tetrazolyl, isoxazolyl, indazolyl, indolyl, benzimidazolyl, pyridazinyl, quinolinyl, purinyl, carbazolyl, acridinyl, pyrimidyl, 2,3′-bifuryl, 3-pyrazolyl and isoquinolinyl groups.

The expression aralkyl refers to groups containing both aryl and also alkyl, alkenyl, alkynyl and/or cycloalkyl groups in accordance with the above definitions, such as, for example, arylalkyl, arylalkenyl, arylalkynyl, arylcycloalkyl, arylcycloalkenyl, alkylarylcycloalkyl and alkylarylcycloalkenyl groups. Specific examples of aralkyls are toluene, xylene, mesitylene, styrene, benzyl chloride, o-fluorotoluene, 1H-indene, tetralin, dihydronaphthalene, indanone, phenylcyclopentyl, cumene, cyclohexylphenyl, fluorene and indan. An aralkyl group preferably contains one or two aromatic ring systems (1 or 2 rings) containing from 6 to 10 carbon atoms and one or two alkyl, alkenyl and/or alkynyl groups containing from 1 or 2 to 6 carbon atoms and/or a cycloalkyl group containing 5 or 6 ring carbon atoms.

The expression heteroaralkyl refers to an aralkyl group as defined above in which one or more (preferably 1, 2, 3 or 4) carbon atoms have been replaced each independently of the others by an oxygen, nitrogen, silicon, selenium, phosphorus, boron or sulphur atom (preferably oxygen, sulphur or nitrogen), that is to say to groups containing both aryl or heteroaryl and also alkyl, alkenyl, alkynyl and/or heteroalkyl and/or cycloalkyl and/or heterocycloalkyl groups in accordance with the above definitions. A heteroaralkyl group preferably contains one or two aromatic ring systems (1 or 2 rings) containing from 5 or 6 to 10 ring carbon atoms and one or two alkyl, alkenyl and/or alkynyl groups containing 1 or 2 to 6 carbon atoms and/or a cycloalkyl group containing 5 or 6 ring carbon atoms, 1, 2, 3 or 4 of those carbon atoms having been replaced each independently of the others by oxygen, sulphur or nitrogen atoms.

Examples are arylheteroalkyl, arylheterocycloalkyl, arylheterocycloalkenyl, arylalkylheterocycloalkyl, arylalkenylheterocycloalkyl, arylalkynylheterocycloalkyl, arylalkylheterocycloalkenyl, heteroarylalkyl, heteroarylalkenyl, heteroarylalkynyl, heteroarylheteroalkyl, heteroarylcycloalkyl, heteroarylcycloalkenyl, heteroarylheterocycloalkyl, heteroarylheterocycloalkenyl, heteroarylalkylcycloalkyl, heteroarylalkylheterocycloalkenyl, heteroarylheteroalkylcycloalkyl, heteroarylheteroalkylcycloalkenyl and heteroarylheteroalkylheterocycloalkyl groups, the cyclic groups being saturated or mono-, di- or tri-unsaturated. Specific examples are a tetrahydroisoquinolinyl, benzoyl, 2- or 3-ethylindolyl, 4-methylpyridino, 2-, 3- or 4-methoxyphenyl, 4-ethoxyphenyl, 2-, 3- or 4-carboxyphenylalkyl group.

The expressions cycloalkyl, heterocycloalkyl, alkylcycloalkyl, heteroalkylcycloalkyl, aryl, heteroaryl, aralkyl and heteroaralkyl can also refer to groups in which one, two or more hydrogen atoms of such groups have been replaced each independently of the others by fluorine, chlorine, bromine or iodine atoms or by OH, ═O, SH, ═S, NH₂, ═NH or NO₂ groups.

The expression “optionally substituted” refers to groups in which one, two or more hydrogen atoms can be replaced each independently of the others by fluorine, chlorine, bromine or iodine atoms or by OH, ═O, SH, ═S, NH₂, ═NH or NO₂ groups. This expression refers furthermore to groups that are substituted by unsubstituted C₁-C₆alkyl, C₂-C₆alkenyl, C₂-C₆alkynyl, C₁-C₆heteroalkyl, C₃-C₁₀cycloalkyl, C₂-C₉heterocycloalkyl, C₆-C₁₀aryl, C₁-C₉heteroaryl, C₇-C₁₂aralkyl or C₂-C₁₁heteroaralkyl groups.

Owing to their substitution, compounds of formulas (I) to (XII) may contain one, two or more centres of chirality. The present invention therefore includes both all pure enantiomers and all pure diastereoisomers and also mixtures thereof in any mixing ratio. The present invention moreover also includes all cis/trans-isomers of the compounds of the general formulas (I) to (XII) and also mixtures thereof. The present invention moreover includes all tautomeric forms of the compounds of formulas (I) to (XII).

Preferred are compounds of formula (I) wherein A is selected from the following groups: —NHCO—, —CH₂CO—, —CH₂SO₂—, —NHSO₂—, —CH₂CH(OH)—, —CH₂CH₂—, —CH(OH)CH₂—, —CONH—, —CH₂N(C₁-C₄-Alkyl)-, —CH₂O— or —CH₂S—.

Especially preferred are compounds of formula (I) wherein A is a group of formula —NHCO— or —CH(OH)CH₂—.

Further preferred are groups of formula (I) having one of the following general structures: Q-NH—CO—R³ or Q-CH(OH)—CH₂—R³.

Also preferred are compounds of formula (I) wherein three, four or five of the groups X¹, X², X³, X⁴, X⁵ and X⁶ each independently of the others are CR² groups.

Especially preferred four of the groups X¹, X², X³, X⁴, X⁵ and X⁶ are each independently of the others CR² groups and two of the groups nitrogen atoms, or five of the groups each independently of the others are CR² groups and one of the groups is a nitrogen atom.

Also preferred are compounds of formula (I) wherein X⁶ is a nitrogen atom.

Moreover preferred are compounds of formula (I) wherein X² and X⁵ are CH groups and X⁴ is a CR² group wherein R² preferably is a hydrogen or a halogen atom.

Further preferred are compounds of formula (I) wherein Q is selected from the following groups:

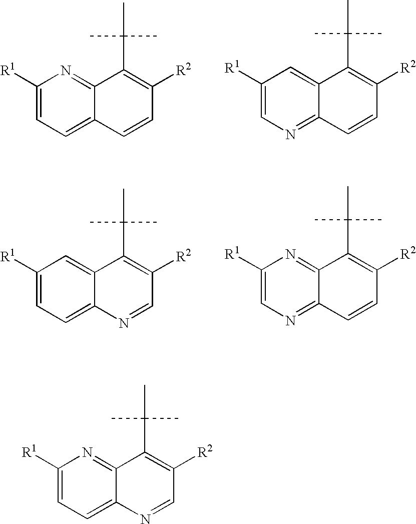

Especially preferred are compounds of formula (I) wherein Q is selected from the following groups:

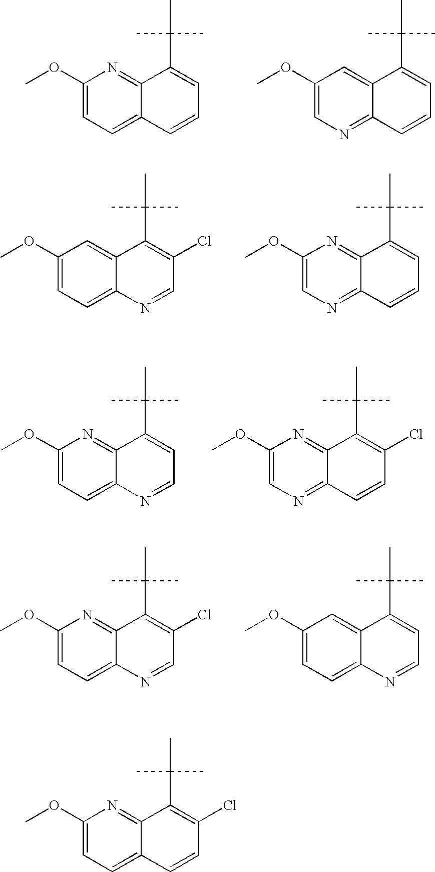

Preferred are compounds of formula (I) wherein R² is a hydrogen atom or a halogen atom; especially preferred R² is a hydrogen atom or a chlorine atom.

Also preferably, R¹ is a C₁-C₄alkyloxy or a C₁-C₄heteroalkyloxy group wherein one or more hydrogen atoms of such groups may have been replaced by fluorine atoms.

Especially preferred are compounds of formula (I) wherein R¹ is a methoxy group.

Preferred are compounds of formula (I) wherein R³ is selected from the following groups:

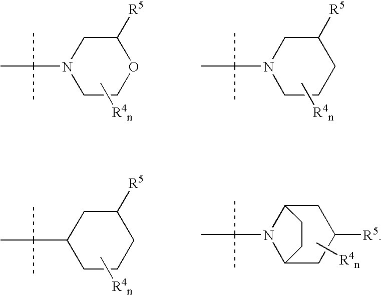

Moreover preferred are compounds of formula (I) wherein R³ is selected from the following groups:

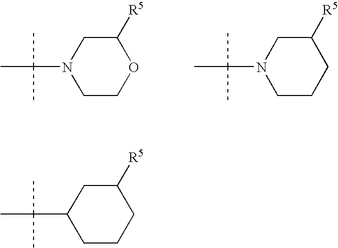

Also preferred is R⁴ a halogen atom, a hydroxy, a C₁-C₄alkyl, a C₁-C₄heteroalkyl or a C₆-C₁₂heteroaralkyl group.

Moreover preferred are compounds of formula (I) wherein n is 0.

Furthermore preferably, R⁵ is a heteroalkylcycloalkyl or a heteroaralkyl group.

R⁵ is especially preferably a group of formula —B—Y, wherein B is an alkylene (especially a C₁-C₄alkylene group), an alkenylene, an alkynylene, a —NH— or a heteroalkylene group (especially a C₁-C₄heteroalkylene group) and Y is an aryl, a heteroaryl, an aralkyl, a heteroaralkyl, a cycloalkyl, a heterocycloalkyl, an alkylcycloalkyl or a heteroalkylcycloalkyl group (especially a heterocycloalkyl, a heteroaryl, an aralkyl, a heteroaralkyl, a heteroarylheterocycloalkyl or an arylheterocyloalkyl group).

Preferably, B is a group of formula —CH₂CH(OH)—, —CH₂NHCH₂—, —CH₂CO—, —NHCH₂CH₂—, —NH—, —CH₂NHCH₂CH₂— or —NHCH₂—.

Especially preferred, B is a group of formula —CH₂NHCH₂— or —NHCH₂—.

Furthermore, Y has preferably one of the following structures: 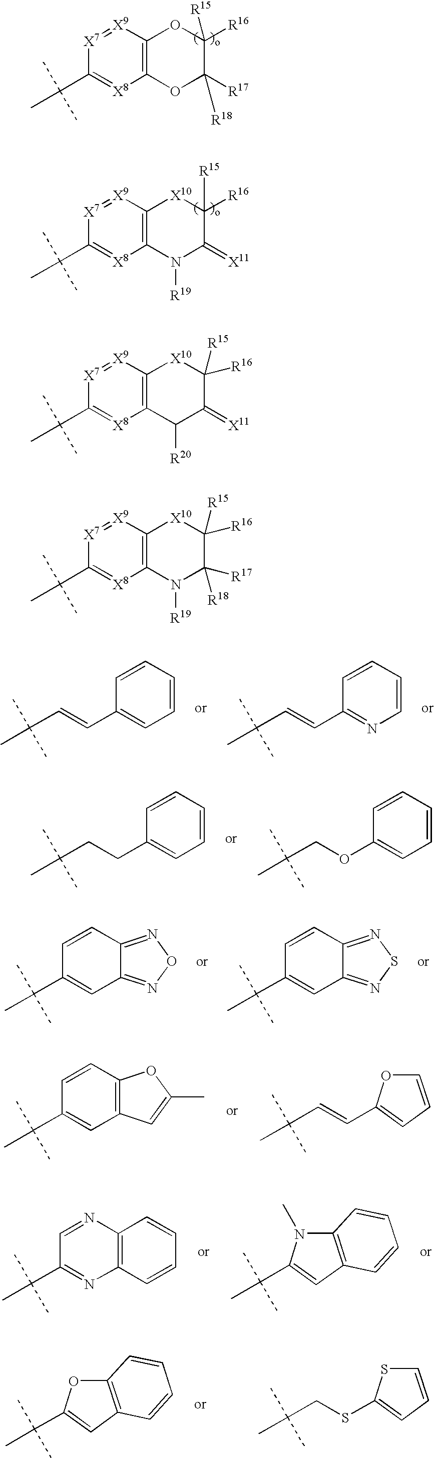 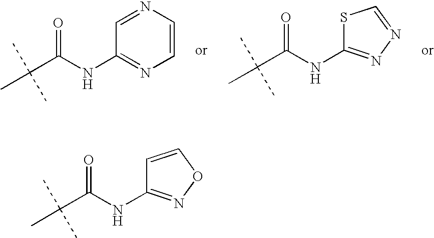
wherein X⁷, X⁸ and X⁹ are each independently of the others nitrogen atoms or groups of formula CR²¹, X¹⁰ and X¹¹ are each independently of the others oxygen or sulphur atoms or groups of formula NR²², o is 0, 1 or 2, R¹⁵, R¹⁶, R¹⁷, R¹⁸, R²⁰ and R²¹ are each independently of the others hydrogen atoms, halogen atoms, hydroxy, alkyl, alkenyl, alkynyl or heteroalkyl groups (especially H, F or Cl) and R¹⁹ and R²² are each independently of the others hydrogen atoms, alkyl, alkenyl, alkynyl or heteroalkyl groups (especially H).

Preferably, Y is selected from one of the following structures:

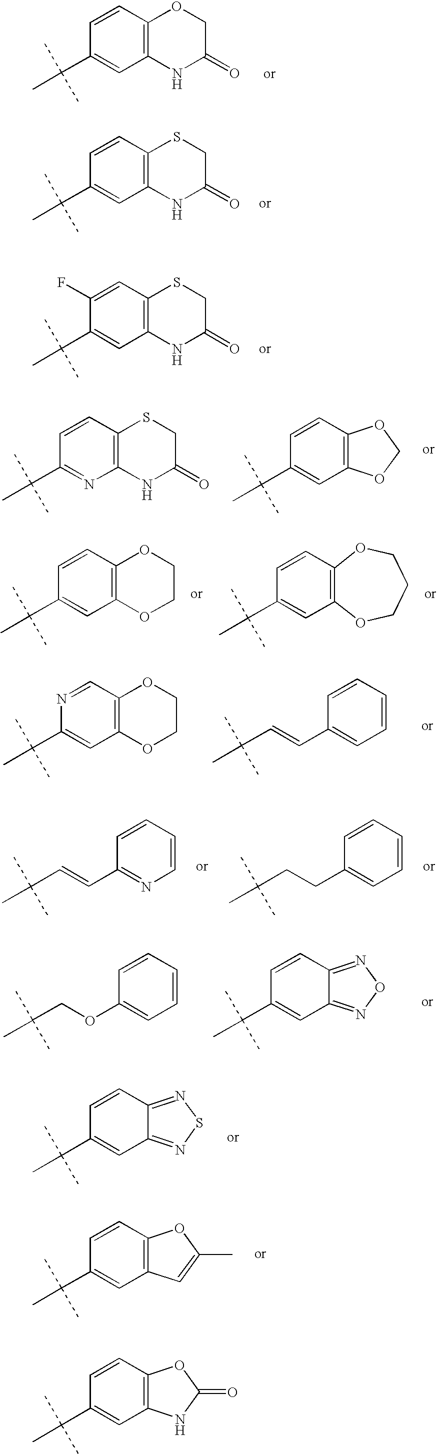

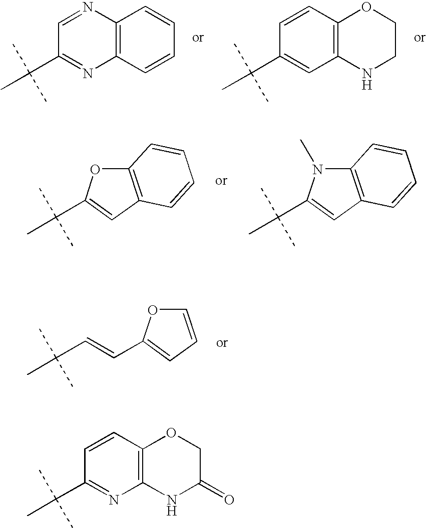

Especially preferably, Y has one of the following structures:

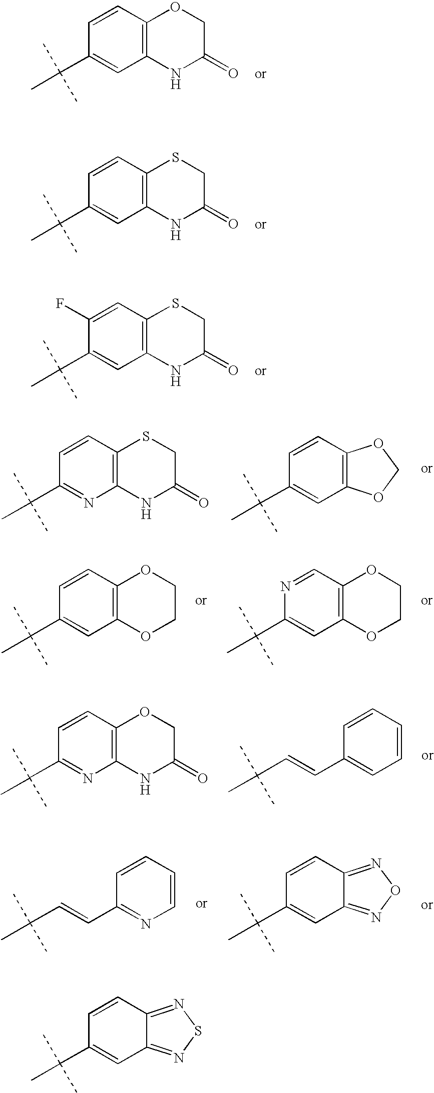

Moreover preferred, Y is selected from one of the following structures:

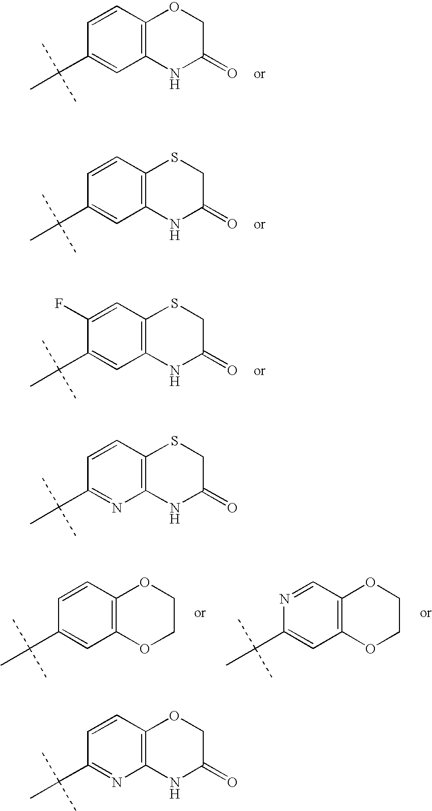

Also preferably, R⁷ is a fluorine or a chlorine atom or a hydroxy, a C₁-C₄alkyloxy or a C₃-C₆dialkylaminomethyl group wherein one or more hydrogen atoms of such groups may have been replaced by fluorine atoms.

Moreover preferred, R⁷ is a hydroxy group.

Especially preferred are compounds of formula (I) having the general structure: Q-NH—CO—R³, wherein Q is selected from the following groups:

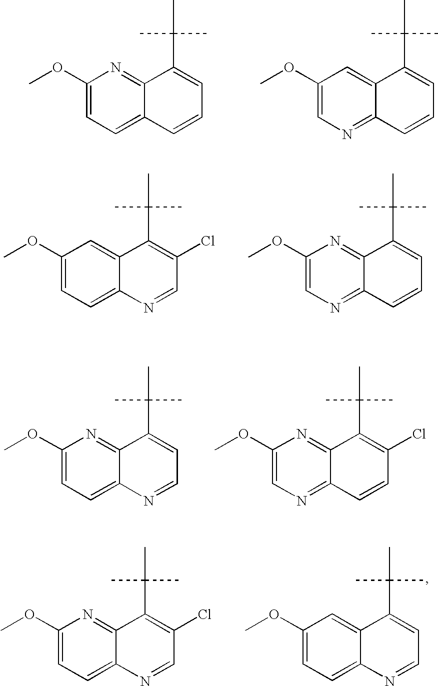

R¹ is selected from the following groups:

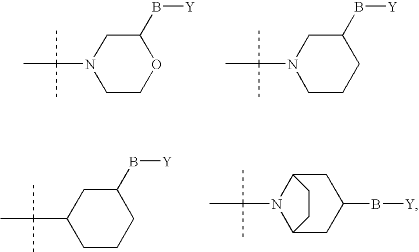

B is a group of formula —CH₂NHCH₂— or —NHCH₂— and Y is defined as above; preferably, Y is selected from the following groups:

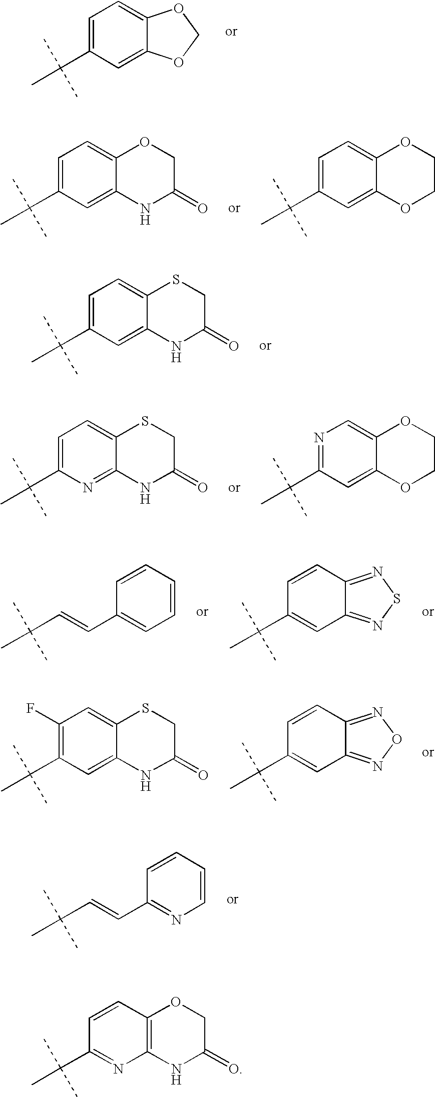

Especially preferred are moreover compounds of formula (I) having the general structure: Q-CH(OH)—CH₂—R³, wherein Q is selected from the following groups:

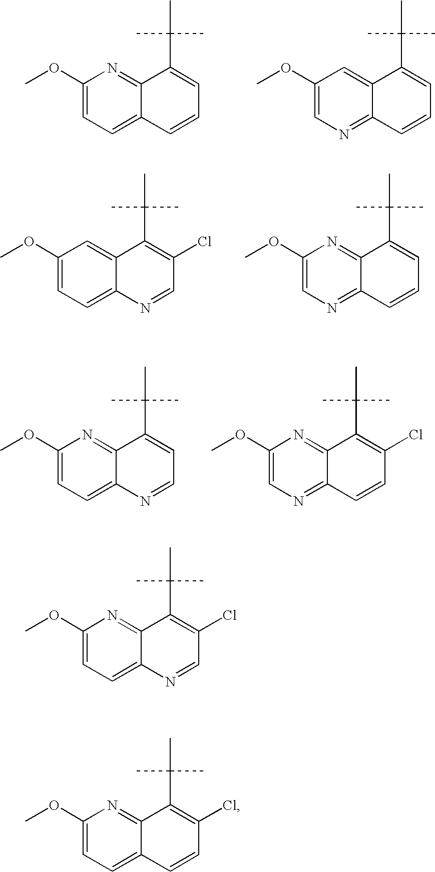

R³ is selected from the following groups:

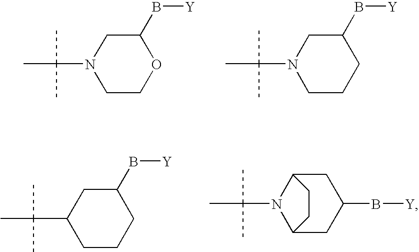

B is a group of formula —CH₂NHCH₂— or —NHCH₂— and Y is defined as above; preferably, Y is selected from the following groups:

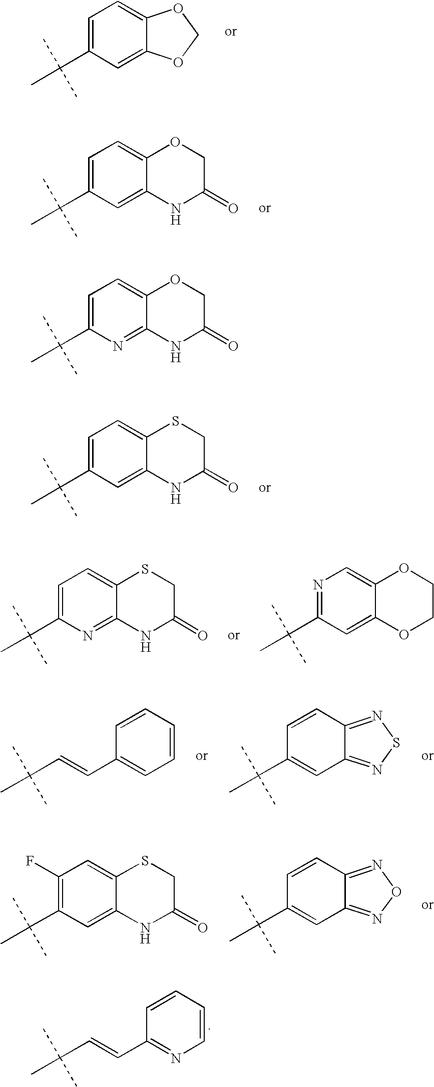

Moreover preferred are compounds of formula (II): 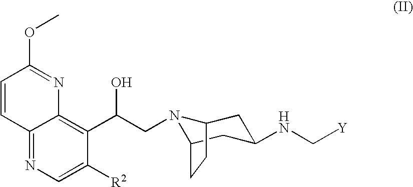
wherein R² is H or a halogen atom (especially H or Cl).

Moreover preferred are compounds of formula (III): 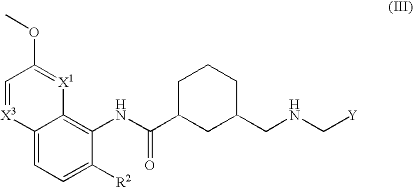
wherein X¹ is N or CH, X³ is N or CH and R² is H or a halogen atom (especially H or Cl), with the proviso that not both X¹ and X³ are CH.

Moreover preferred are compounds of formula (IV): 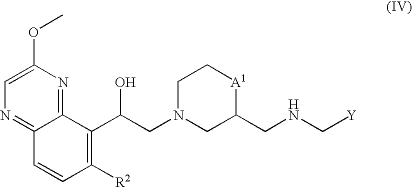
wherein A¹ is CH₂ or O and R² is H or a halogen atom (especially H or Cl).

Moreover preferred are compounds of formula (V): 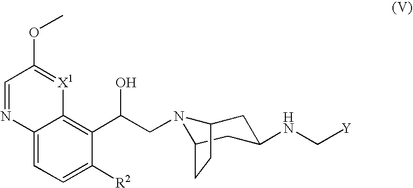
wherein X¹ is N or CH and R² is H or a halogen atom (especially H or Cl).

Moreover preferred are compounds of formula (VI): 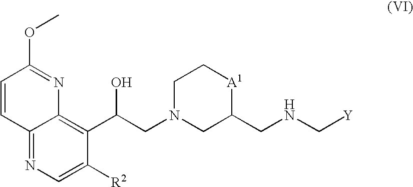
wherein A¹ is CH₂ or O and R² is H or a halogen atom (especially H or Cl).

Moreover preferred are compounds of formula (VII): 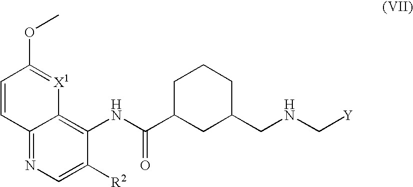
wherein X¹ is N or CH and R² is H or a halogen atom (especially H or Cl).

Moreover preferred are compounds of formula (VIII): 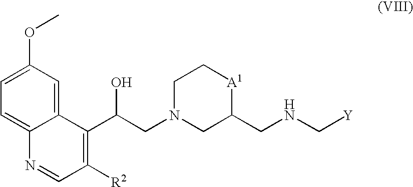
wherein A¹ is O or CH₂ and R² is a halogen atom (especially Cl).

Moreover preferred are compounds of formula (IX): 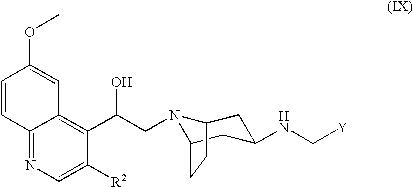
wherein R² is a halogen atom (especially Cl).

Moreover preferred are compounds of formula (X): 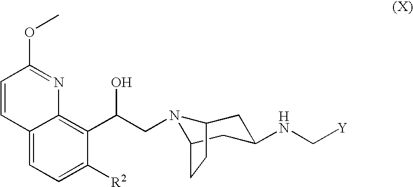
wherein R² is H or a halogen atom (especially H or Cl).

Moreover preferred are compounds of formula (XI): 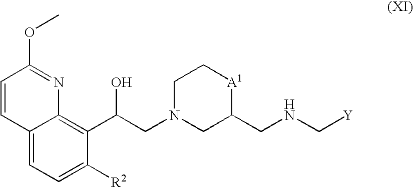
wherein A¹ is CH₂ or O and R² is H or a halogen atom (especially H or Cl).

In formulas (II), (III), (IV), (V), (VI), (VII), (VIII), (IX), (X) and (XI), Y is defined as above.

Especially preferred are compounds of formulas (II), (III), (IV), (V), (VI), (VII), (VIII), (IX), (X) and (XI) wherein Y is selected from the following groups:

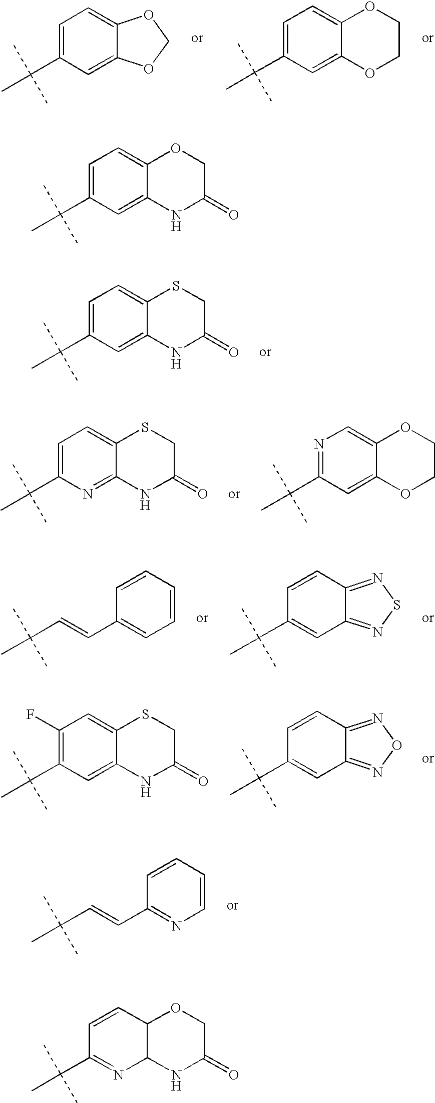

Moreover preferred are compounds of formula (XII): 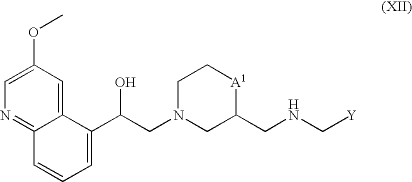
wherein A¹ is O or CH₂ and Y is selected from the following groups:

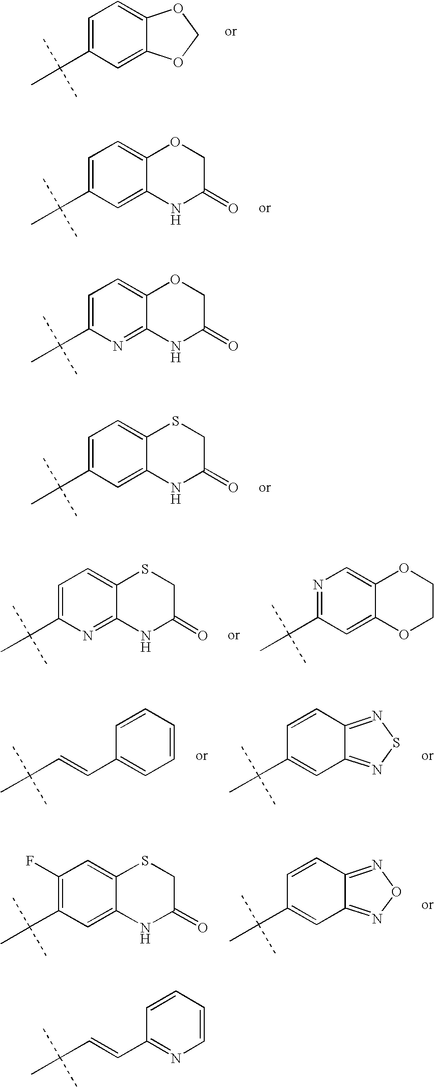

It is especially preferred to combine the preferred embodiments for each generic group in formula (I) in any possible manner.

The therapeutic use of compounds of formulas (I) to (XII), their pharmacologically acceptable salts or solvates and hydrates and also formulations and pharmaceutical compositions also lie within the scope of the present invention.

The pharmaceutical compositions according to the present invention comprise at least one compound of formulas (I) to (XII) and, optionally, carrier substances and/or adjuvants.

Examples of pharmacologically acceptable salts of the compounds of formulas (I) to (XII) are salts of physiologically acceptable mineral acids, such as hydrochloric acid, sulphuric acid and phosphoric acid, or salts of organic acids, such as methanesulphonic acid, p-toluenesulphonic acid, lactic acid, acetic acid, trifluoroacetic acid, citric acid, succinic acid, fumaric acid, maleic acid and salicylic acid. Further examples of pharmacologically acceptable salts of the compounds of formulas (I) to (XII) are alkali metal and alkaline earth metal salts such as, for example, sodium, potassium, lithium, calcium or magnesium salts, ammonium salts or salts of organic bases such as, for example, methylamine, dimethylamine, triethylamine, piperidine, ethylenediamine, lysine, choline hydroxide, meglumine, morpholine or arginine salts. Compounds of formulas (I) to (XII) may be solvated, especially hydrated. The hydration may take place, for example, during the preparation process or as a consequence of the hygroscopic nature of the initially anhydrous compounds of formulas (I) to (XII). When the compounds of formulas (I) to (XII) comprise asymmetric C-atoms, they may be present either in the form of achiral compounds, diastereoisomeric mixtures, mixtures of enantiomers or in the form of optically pure compounds.

The pro-drugs to which the present invention also relates consist of a compound of formulas (I) to (XII) and at least one pharmacologically acceptable protecting group which will be removed under physiological conditions, such as, for example, an alkoxy-, aralkyloxy-, acyl- or acyloxy group, such as, for example, an ethoxy, benzyloxy, acetyl or acetyloxy.

The present invention relates also to the use of those active ingredients in the preparation of medicaments. In general, compounds of formulas (I) to (XII) are administered either individually, or in combination with any other desired therapeutic agent, using the known and acceptable methods. Such therapeutically useful agents may be administered, for example, by one of the following routes: orally, for example in the form of dragées, coated tablets, pills, semi-solid substances, soft or hard capsules, solutions, emulsions or suspensions; parenterally, for example in the form of an injectable solution; rectally in the form of suppositories; by inhalation, for example in the form of a powder formulation or a spray; transdermally or intranasally. For the preparation of such tablets, pills, semi-solid substances, coated tablets, dragées and hard gelatine capsules, the therapeutically usable product may be mixed with pharmacologically inert, inorganic or organic pharmaceutical carrier substances, for example with lactose, sucrose, glucose, gelatine, malt, silica gel, starch or derivatives thereof, talcum, stearic acid or salts thereof, skimmed milk powder, and the like. For the preparation of soft capsules, pharmaceutical carrier substances such as, for example, vegetable oils, petroleum, animal or synthetic oils, wax, fat and polyols may be used. For the preparation of liquid solutions and syrups, pharmaceutical carrier substances such as, for example, water, alcohols, aqueous saline solution, aqueous dextrose, polyols, glycerol, vegetable oils, petroleum and animal or synthetic oils may be used. For suppositories, pharmaceutical carrier substances such as, for example, vegetable oils, petroleum, animal or synthetic oils, wax, fat and polyols may be used. For aerosol formulations, compressed gases that are suitable for this purpose, such as, for example, oxygen, nitrogen and carbon dioxide may be used. The pharmaceutically acceptable agents may also comprise additives for preserving and stabilising, emulsifiers, sweeteners, flavourings, salts for altering the osmotic pressure, buffers, encapsulation additives and antioxidants.

The compounds of formulas (I), (II), (III), (IV), (V), (VI), (VII), (VIII), (IX), (X), (XI) and (XII) have improved properties when compared to antibacterial compounds known in the state of the art, especially, improved antibacterial activity, improved solubility and improved PK properties.

Combinations with other therapeutic agents which are also encompassed by the present invention may comprise one, two or more other antimicrobial and anti-fungal active ingredients.

For the prevention and/or treatment of bacterial infections, the dose of the biologically active compound according to the invention may vary within wide limits and may be adjusted to individual requirements. Generally, a dose of from 10 mg to 4000 mg per day is suitable, a preferred dose being from 50 to 3000 mg per day. In suitable cases, the dose may also be below or above the stated values. The daily dose may be administered as a single dose or in a plurality of doses. A typical individual dose contains approximately 50 mg, 100 mg, 250 mg, 500 mg, 1 g or 2 g of the active ingredient.

### EXAMPLES

### Example 1

### 6-[({1-[2-Hydroxy-2-(6-methoxy-[1,5]-naphthyridin-4-yl)-ethyl]-piperidin-3-ylmethyl}-amino)-methyl]-4H-benzo[1,4]oxazin-3-one (enantiomer 1)

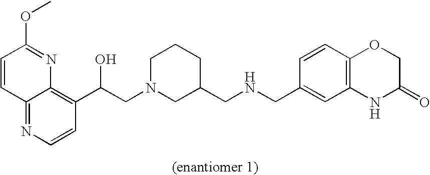

### 1a) 6-Methoxy-[1,5]-naphthyridin-4-ol

5-Amino-2-methoxypyridine (12.29 g) was dissolved in ethanol (41 ml) and treated with 2,2-dimethyl-[1,3]dioxane-4,6-dione (17 g) and triethyl orthoformate (17 ml). The mixture was refluxed for 3 hours and then cooled to room temperature. The precipitate was filtered off, washed with ethanol and dried under vacuum for 1 hour to give 25.24 g of the intermediate.

The intermediate was added to refluxing diphenyl ether (292 g) (260° C.) slowly in portions. The mixture was stirred at 260° C. until the gas evolution had ceased (ca. 3 minutes) and then cooled in an ice bath. The precipitated solid was suspended in diethyl ether and filtered. The solid washed with cold diethyl ether and ethyl acetate to give the desired product (13.2 g).

¹H NMR (300 MHz, d₆-DMSO): δ: 11.90 (bs, 1H), 7.96-7.89 (m, 2H), 7.16 (d, 1H), 6.20 (bs, 1H), 3.93 (s, 3H)

### 1b) Trifluoro-methanesulfonic acid 6-methoxy-[1,5]-naphthyridin-4-yl ester

Naphthyridin-4-ol (1a) (4.83 g) was suspended in dichloromethane (111 ml), cooled to 0° C. and treated with 2,6-lutidine (4.8 ml), DMAP (0.50 g) and trifluoromethanesulfonic anhydride (5.1 ml). The mixture was stirred at this temperature for 4 hours, then diluted with saturated ammonium chloride solution and extracted twice with dichloromethane. The organic layer washed with brine, dried over magnesium sulfate, filtered and evaporated. The residue was purified by flash chromatography (silica gel, dichloromethane) to give the desired product (6.14 g).

¹H NMR (300 MHz, CDCl₃): δ: 8.85 (d, 1H), 8.18 (d, 1H), 7.35 (d, 1H), 7.17 (d, 1H), 4.06 (s, 3H)

### 1c) 2-Methoxy-8-vinyl-[1,5]-naphthyridine

Triflate (1b) (10.00 g) and tributyl vinyl stannane (10.4 ml) were dissolved in dry DMF (173 ml) and degassed by bubbling argon through for 25 minutes. Then PdCl₂(PPh₃)₂ (1.14 g) was added and the mixture stirred at 90° C. over night. The DMF was evaporated and the residue dissolved in diethyl ether. The suspension was filtered over Celite® and the filtrate washed with water, saturated potassium fluoride solution and brine. The organic layer was dried over magnesium sulfate, filtered and evaporated. The residue was purified by flash chromatography (silica gel, hexane/ethyl acetate) to give the desired product (4.34 g).

¹H NMR (300 MHz, CDCl₃): δ: 8.72 (d, 1H), 8.19 (d, 1H), 7.80 (dd, 1H), 7.64 (d, 1H), 6.22 (dd, 1H), 5.55 (dd, 1H), 4.10 (s, 3H)

### 1d) 1-(6-Methoxy-[1,5]-naphthyridin-4-yl)-ethane-1,2-diol (enantiomer 1)

Vinyl-naphthyridine (1c) (4.34 g) was dissolved in water (144 ml) and tert. butanol (144 ml), treated with AD mix beta (41.5 g) and stirred at 0° C. for 2 days. To the mixture was added sodium metabisulfite (30.47 g) at 0° C. and then stirred for 60 minutes at this temperature. The mixture was filtered and the filtrate evaporated. The residue was dissolved in water and extracted twice with ethyl acetate. The combined organic layers were washed with brine, dried over magnesium sulfate, filtered and concentrated. The residue was purified by flash chromatography (silica gel, ethyl acetate) to give the desired product (3.82 g).

¹H NMR (300 MHz, CDCl₃): δ: 8.62 (d, 1H), 8.17 (d, 1H), 7.59 (d, 1H), 7.08 (d, 1H), 5.52-5.48 (m, 1H), 4.08 (dd, 1H), 4.00 (s, 3H), 3.92 (dd, 1H)

### 1e) Toluene-4-sulfonic acid 2-hydroxy-2-(6-methoxy-[1,5]-naphthyridin-4-yl)-ethyl ester (enantiomer 1)

Diol (1d) (3.82 g) was suspended in dichloromethane (150 ml), triethylamine (12 ml) and THF (30 ml). DMAP (318 mg) was added, the mixture cooled to −78° C. and stirred for 5 minutes. Then 4-toluene sulfonyl chloride (3.31 g) was added. The mixture stirred for 2.5 hours at −78° C. and then placed in the freezer over night. The mixture was diluted with dichloromethane and washed with water and brine. The organic layer was dried over magnesium sulfate, filtered and concentrated. The residue was purified by flash chromatography (silica gel, ethyl acetate/hexane 1:1) to give the desired product (2.11 g).

MS (EI): m/z: 375 [M+H]⁺

### 1f) 2-Methoxy-8-oxiranyl-[1,5]-naphthyridine (enantiomer 1)

The tosylate (1e) (2.11 g) was dissolved in DMF (10 ml), cooled to 0° C. and stirred at this temperature for 10 minutes. Then sodium hydride (225 mg) was added, the mixture stirred for 15 minutes at 0° C. and stirred over night at room temperature. The mixture was diluted with diethyl ether and washed with water and brine. The organic layer was dried over magnesium sulfate, filtered and concentrated. The residue was purified by flash chromatography (silica gel, ethyl acetate/hexane 1:1, 3:7) to give the desired product (1.16 g).

¹H NMR (300 MHz, CDCl₃): δ: 8.75 (d, 1H), 8.26 (d, 1H), 7.39 (d, 1H), 7.19-7.13 (m, 1H), 4.96 (m, 1H), 4.10 (s, 3H), 3.38 (m, 1H), 2.82 (m, 1H)

### 1g) {1-[2-Hydroxy-2-(6-methoxy-[1,5]-naphthyridin-4-yl)-ethyl]-piperidin-3-ylmethyl}-carbamic acid tert-butyl ester (enantiomer 1)

To a solution of the epoxide (1f) (1.16 g) and piperidin-3-ylmethyl-carbamic acid tert.-butyl ester (1.48 g) in DMF (10 ml) was added lithium perchlorate (116 mg) and the mixture was stirred at reflux for 3 hours. The mixture was dissolved in water (150 ml), extracted three times with ethyl acetate. The combined organic layers were washed with brine, dried over magnesium sulfate, filtered and evaporated. The crude product was purified by flash chromatography (silica gel, dichloromethane/methanol 9:1) to give the desired product (1.1 g).

¹H NMR (300 MHz, CDCl₃): δ: 8.70 (d, 1H), 8.14 (d, 1H), 7.74 (d, 1H), 7.03 (d, 1H), 5.81 (bd, 1H), 4.71-4.55 (m, 1H), 3.96 (s, 3H), 3.26-3.10 (m, 1H), 3.07-2.84 (m, 4H), 2.68-2.46 (m, 1H), 2.34-1.92 (m, 2H), 1.89-1.45 (m, 5H), 1.35 (s, 9H), 1.11-0.95 (m, 1H)

### 1h) 2-(3-Aminomethyl-piperidin-1-yl)-1-(6-methoxy-[1,5]-naphthyridin-4-yl)-ethanol (enantiomer 1)

To a solution of Boc-amine (1g) (1.0 g) in dichloromethane (20 ml), was added TFA (10 ml) and stirred for 20 minutes at room temperature. The mixture was concentrated and dichloromethane (20 ml) and 2N aqueous sodium hydroxide solution (40 ml) added. The layers were separated and the aqueous layer was extracted three times with dichloromethane. The combined organic layers were dried over magnesium sulfate, filtered and evaporated. The crude product was purified by flash chromatography (silica gel, dichloromethane/methanol 9:1) to give the desired product (0.9 g).

¹H NMR (300 MHz, CDCl₃): δ: 8.73-8.68 (m, 1H), 8.20-8.13 (m, 1H), 7.74-7.69 (m, 1H), 7.12-7.03 (m, 1H), 5.95 (bs, 2H), 5.81 (bd, 1H), 4.00 (s, 3H), 3.48 (s, 2H), 3.05-2.74 (m, 3H), 2.65-2.40 (m, 2H), 2.34-2.18 (m, 1H), 2.16-1.98 (m, 1H), 1.88-1.55 (m, 4H), 1.35-1.15 (m, 1H)

### 1i) (4-Formyl-2-nitro-phenoxy)-acetic acid ethyl ester

Potassium carbonate (22.7 g) was added to a stirred solution of 4-Hydroxy-3-nitrobenzaldehyde (25 g) in DMF (250 ml). Chloroacetic acid ethyl ester (23.2 ml) was added dropwise and the mixture was stirred for 2 days at 50° C. and another 2 days at room temperature. The mixture was diluted with water and extracted with ethyl acetate. The combined organic layers were washed with water, dried over magnesium sulfate, filtered and evaporated to give the desired compound (37.8 g).

¹H NMR (300 MHz, d₆-DMSO): δ: 9.96 (s, 1H), 8.44 (s, 1H), 8.15 (dd, 1H), 7.52 (d, 1H), 5.17 (s, 2H), 4.18 (q, 2H), 1.21 (t, 3H)

### 1j) 3-Oxo-3,4-dihydro-2H-benzo[1,4]oxazine-6-carbaldehyde

Iron powder (83 g) was added to a stirred solution of compound (1i) (37.7 g) dissolved in acetic acid (1 l) and the mixture stirred for 1.5 hours at 80° C. The reaction mixture was filtered through Decalite and concentrated. The residue was dissolved in saturated sodium bicarbonate and extracted with ethyl acetate. The combined organic layers were dried over magnesium sulfate, filtered and evaporated. The residue was triturated with diethyl ether and the precipitate filtered off to give the desired product (20 g).

¹H NMR (300 MHz, d₆-DMSO): δ: 11.00 (bs, 1H), 9.84 (s, 1H), 7.54 (dd, 1H), 7.39 (d, 1H), 7.14 (d, 1H), 4.72 (s, 2H)

### 1k) Title Compound

Amine (1h) (100 mg) was dissolved in 1,2-dichloroethane (6 ml) and methanol (2 ml), treated with sieves 3A (1.00 g) and aldehyde (1j) (67 mg) and stirred at room temperature over night. Then sodium borohydride (12 mg) was added and the mixture stirred at room temperature over night. The sieves were filtered off and the filtrate washed with saturated sodium bicarbonate and brine. The organic layer was dried over magnesium sulfate, filtered and concentrated. The residue was purified by flash chromatography (silica gel, ethyl acetate/methanol 9:1+1% ammonia) to give the desired product (70 mg).

¹H NMR (300 MHz, d₆-DMSO): δ: 10.65 (s, 1H), 8.75 (d, 1H), 8.25 (d, 1H), 7.75 (d, 1H), 7.24 (d, 1H), 6.93-6.78 (m, 3H), 5.80-5.77 (m, 2H), 5.22 (bs, 1H), 4.54 (s, 2H), 3.99 (s, 3H), 3.59-3.55 (m, 2H), 3.33-3.23 (m, 2H), 3.07-3.03 (m, 1H), 2.92-2.79 (m, 1H), 2.73-2.64 (m, 1H), 2.46-2.40 (m, 1H), 2.38-2.25 (m, 2H), 2.15-2.08 (m, 1H), 1.69-1.38 (m, 5H)

### Example 2

### 2-(3-{[(Benzo[1,3]dioxol-5-ylmethyl)-amino]-methyl}-piperidin-1-yl)-1-(6-methoxy-[1,5]-naphthyridin-4-yl)-ethanol (enantiomer 1)

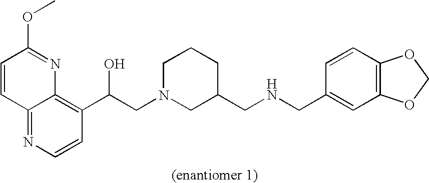

The compound was prepared as in example 1k from benzo[1,3]dioxole-5-carbaldehyde.

¹H NMR (300 MHz, d₆-DMSO): δ: 8.72-8.62 (m, 1H), 8.19-8.08 (m, 1H), 7.73-7.59 (m, 1H), 7.06-6.99 (m, 1H), 6.92-6.68 (m, 3H), 5.86-5.74 (m, 1H), 3.94 (s, 3H), 3.86-3.80 (m, 2H), 3.18-2.92 (m, 4H), 2.62-2.55 (m, 4H), 2.25-1.88 (m, 4H), 1.80-1.40 (m, 4H), 1.12-0.92 (m, 1H)

### Example 3

### 2-(3-{[(2,3-Dihydro-benzo[1,4]dioxin-6-ylmethyl)-amino]-methyl}-piperidin-1-yl)-1-(6-methoxy-[1,5]-naphthyridin-4-yl)-ethanol (enantiomer 1)

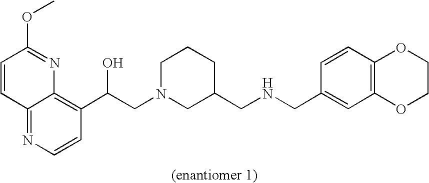

The compound was prepared as in example 1k from 2,3-dihydro-benzo[1,4]dioxine-6-carbaldehyde.

¹H NMR (300 MHz, d₆-DMSO): δ: 8.69 (d, 1H), 8.12 (d, 1H), 7.72 (d, 1H), 7.02 (d, 1H), 6.78-6.65 (m, 3H), 5.72-5.64 (m, 1H), 4.15 (s, 3H), 4.00-3.91 (m, 2H), 3.66-3.58 (m, 2H), 3.28-3.18 (m, 1H), 3.16-3.08 (m, 1H), 3.02-2.89 (m, 2H), 2.84-2.72 (m, 1H), 2.70-2.58 (m, 1H), 2.54-2.39 (m, 3H), 2.38-2.20 (m, 1H), 2.11-1.98 (m, 1H), 1.94-1.48 (m, 5H), 1.04-0.84 (m, 1H)

### Example 4

### 2-(3-{[(Benzo[1,2,5]thiadiazol-5-ylmethyl)-amino]-methyl}-piperidin-1-yl)-1-(6-methoxy-[1,5]-naphthyridin-4-yl)-ethanol (enantiomer 1)

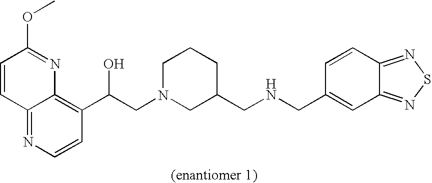

The compound was prepared as in example 1k from benzo[1,2,5]thiadiazole-5-carbaldehyde.

¹H NMR (300 MHz, d₆-DMSO): δ: 8.77-8.72 (m, 1H), 8.25 (d, 1H), 8.04-7.91 (m, 2H), 7.76-7.61 (m, 2H), 7.26-7.22 (m, 1H), 5.81-5.78 (m, 1H), 3.98 (s, 3H), 3.85 (s, 2H), 3.34-3.27 (m, 1H), 3.08-2.92 (m, 1H), 2.83-2.65 (m, 2H), 2.46-2.30 (m, 3H), 2.15-2.04 (m, 1H), 1.92-1.43 (m, 7H)

### Example 5

### 1-(6-Methoxy-[1,5]-naphthyridin-4-yl)-2-{3-[((E)-3-pyridin-2-yl-allylamino)-methyl]-piperidin-1-yl}-ethanol (enantiomer 1)

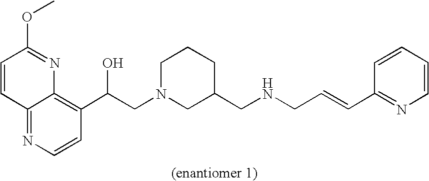

### 5a) (E)-3-Pyridin-2-yl-propenal

To a solution of formylpyridine (4.22 g) in toluene (400 ml) was added (triphenyl-lambda*5*-phosphanylidene)-acetaldehyde (12 g). The mixture was stirred for 2 days at room temperature and evaporated. The crude product was purified by flash chromatography (silica gel, hexane/ethyl acetate 2:1, 1:1; 1:2) to give the desired product (3.96 g).

¹H NMR (300 MHz, CDCl₃): δ: 9.74 (d, 1H), 8.66-8.64 (m, 1H), 7.78-7.73 (m, 1H), 7.57-7.47 (m, 2H), 7.33-7.23 (m, 1H), 7.07-7.00 (m, 1H).

### 5b) Title Compound

The compound was prepared as in example 1k from aldehyde (5a).

¹H NMR (300 MHz, d₆-DMSO): δ: 8.55-8.51 (m, 1H), 8.30-8.27 (m, 1H), 8.03 (d, 1H), 7.55-7.48 (m, 2H), 7.26-7.21 (m, 1H), 7.08-6.98 (m, 2H), 6.55-6.37 (m, 1H), 5.61-5.57 (m, 1H), 3.79 (s, 3H), 3.21-3.05 (m, 3H), 2.90-2.40 (m, 4H), 2.08-1.60 (m, 3H), 1.52-1.15 (m, 5H), 1.10-0.88 (m, 3H).

### Example 6

### 6-[({1-[2-Hydroxy-2-(6-methoxy-[1,5]-naphthyridin-4-yl)-ethyl]-piperidin-3-ylmethyl}-amino)-methyl]-4H-benzo[1,4]thiazin-3-one (enantiomer 1)

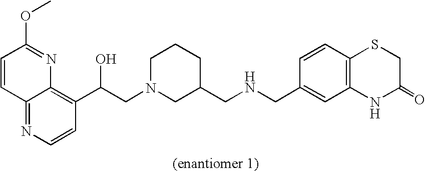

### 6a) (4-Formyl-2-nitro-phenylsulfanyl)-acetic acid methyl ester

4-Chloro-3-nitrobenzaldehyde (10 g) was dissolved in DMF (100 ml), sodium hydride (2.35 g) was added and the mixture stirred for 15 minutes. Then methyl thioglycolate (3.45 ml) was added dropwise and the mixture stirred for 5 hours at room temperature. The reaction mixture was diluted with water and extracted with ethyl acetate. The combined organic layers were washed twice with water, dried over sodium sulfate, filtered and evaporated. The crude product was purified by flash chromatography (silica gel, hexane/ethyl acetate 2:1) to give the desired product (5.5 g).

¹H NMR (300 MHz, CDCl₃): δ: 10.05 (s, 1H), 8.75 (d, 1H), 8.09 (dd, 1H), 7.68 (d, 1H), 3.84 (s, 2H), 3.81 (s, 3H)

### 6b) 3-Oxo-3,4-dihydro-2H-benzo[1,4]thiazine-6-carbaldehyde

Compound (6a) (5.5 g) was dissolved in acetic acid (115 ml), then iron powder (8.42 g) was added in portions. The mixture was stirred for 15 minutes at room temperature, then 3 hours at 50° C. The mixture was cooled, filtered through decalite, the filter cake washed with methanol and the filtrate and washings were evaporated. The residue was dissolved in saturated sodium bicarbonate and extracted with ethyl acetate. The combined organic layers were dried over sodium sulfate, filtered and evaporated. The crude product was purified by flash chromatography (silica gel, hexane/ethyl acetate 2:1, ethyl acetate) to give the desired product (1 g).

¹H NMR (300 MHz, CDCl₃): δ: 10.18 (bs, 1H), 9.85 (s, 1H), 7.45-7.34 (m, 3H), 3.39 (s, 2H)

### 6c) Title Compound

The compound was prepared as in example 1k from aldehyde (6b).

¹H NMR (300 MHz, d₆-DMSO): δ: 10.54-10.51 (m, 1H), 8.78-8.74 (m, 1H), 8.25 (d, 1H), 7.76 (d, 1H), 7.27-7.20 (m, 2H), 6.95-6.89 (m, 2H), 4.07-3.99 (m, 4H), 3.58 (s, 2H), 3.43 (s, 2H), 3.42 (bs, 2H), 3.14-3.06 (m, 1H), 2.95-2.64 (m, 3H), 2.46-2.41 (m, 1H), 2.35-2.27 (m, 2H), 2.13-2.07 (m, 1H), 2.00-1.97 (m, 2H), 1.89-1.39 (m, 4H)

### Example 7

### 1-(6-Methoxy-[1,5]-naphthyridin-4-yl)-2-{3-[((E)-3-phenyl-allylamino)-methyl]-piperidin-1-yl}-ethanol (enantiomer 1)

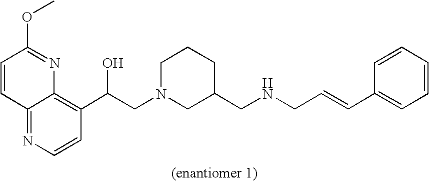

The compound was prepared as in example 1k from cinnamic aldehyde.

¹H NMR (300 MHz, d₆-DMSO): δ: 8.78-8.73 (m, 1H), 8.25 (d, 1H), 7.78-7.70 (m, 1H), 7.47-7.29 (m, 6H), 6.71-6.64 (m, 1H), 6.38-6.26 (m, 1H), 5.84-5.78 (m, 1H), 4.00 (s, 3H), 3.59-3.51 (m, 3H), 3.36-2.94 (m, 2H), 2.86-2.62 (m, 3H), 2.17-1.40 (m, 7H), 1.08-0.92 (m, 2H)

### Example 8

### 6-({8-[2-Hydroxy-2-(6-methoxy-[1,5]-naphthyridin-4-yl)-ethyl]-8-aza-bicyclo[3.2.1]oct-3-ylamino}-methyl)-4H-benzo[1,4]oxazin-3-one (enantiomer 1)

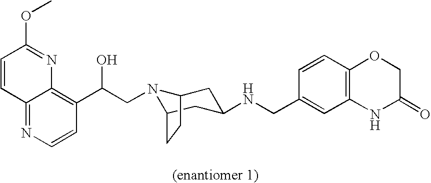

### 8a) 8-Benzyl-8-aza-bicyclo[3.2.1]octan-3-one

To 10% hydrochloric acid solution (206 ml) was added 2,3-dimethoxytetrahydrofuran (50 ml). The mixture was mechanically stirred at room temperature for 20 minutes. After cooling to 0° C., a solution of benzylamine (50.7 ml) in water (250 ml) and 6N hydrochloric acid solution (78 ml) was added followed by 1,3-acetone dicarboxylic acid (56.4 g) and 10% sodium acetate solution (175 ml). The mixture was stirred 5 minutes at the same temperature and then 1 hour at room temperature. The reaction mixture was then heated at 50° C. for 2 hours. After cooling, the heterogeneous mixture was filtered and the cake discarded. The filtrate washed three times with diethyl ether (3×200 ml). The pH of the aqueous layer was adjusted to 7 by adding solid sodium bicarbonate and extracted with ethyl acetate (3×400 ml). The combined extracts were washed with brine and dried over magnesium sulfate. After concentration to dryness, the residue was purified by flash chromatography (silica gel, ethyl acetate/hexane 3:7, 1:1) to afford the desired compound (20 g).

¹H NMR (300 MHz, CDCl₃): δ: 7.26-6.94 (m, 5H), 3.57 s, 2H), 2.55-2.48 (dd, 2H), 2.06-1.88 (m, 4H), 1.54-1.37 (m, 2H)

### 8b) 8-Benzyl-8-aza-bicyclo[3.2.1]octan-3-ol

To a solution of ketone (8a) (16.8 g) in THF (95 ml) cooled to −78° C., was added L-selectride (94 ml). The reaction mixture was stirred for 90 minutes, warmed to room temperature and stirred for 1 hour at room temperature. The mixture then was cooled to 0° C., 20% sodium hydroxide solution (81 ml) were added, then 30% hydrogen peroxide (41 ml) and stirred for 1 hour at room temperature. The aqueous layer was extracted three times with dichloromethane. The combined organic layers were washed with brine, dried over magnesium sulfate, filtered and evaporated. The crude material was purified by flash chromatography (silica gel, dichloromethane/(methanol/ammonia 9:1) 19:1, dichloromethane/methanol 9:1+1% ammonia) to give the desired product (8.12 g).

¹H NMR (300 MHz, CDCl₃): δ: 7.34-7.06 (m, 5H), 4.05-3.95 (m, 1H), 3.49 (s, 2H), 3.19-3.05 (m, 2H), 2.13-1.90 (m, 6H), 1.68-1.52 (m, 2H), 1.38-1.21 (m, 1H)

### 8c) Methanesulfonic acid 8-benzyl-8-aza-bicyclo[3.2.1]oct-3-yl ester

To a solution of alcohol (8b) (8.0 g) in dichloromethane (132 ml) cooled to 0° C., were added triethylamine (10 ml) and methane sulfonyl chloride (3.5 ml). The reaction mixture was stirred at the same temperature for 60 minutes and at room temperature over night. Water was added and the mixture extracted twice with dichloromethane. The combined organic layers were washed with brine, dried over magnesium sulfate, filtered and concentrated. The residue was purified by flash chromatography (silica gel, ethyl acetate/methanol 9:1) to give the desired product (9.84 g).

¹H NMR (300 MHz, CDCl₃): δ: 7.30-7.18 (m, 5H), 4.92-4.88 (m, 1H), 3.54-3.43 (m, 2H), 3.21-3.08 (m, 2H), 2.91 (s, 3H), 2.30-2.12 (m, 2H), 2.05-1.95 (m, 6H)

### 8d) 3-Azido-8-benzyl-8-aza-bicyclo[3.2.1]octane

To a solution of mesylate (8c) (9.84 g) in DMF (111 ml) was added sodium azide (6.49 g). The reaction mixture was heated at 65° C. for 14 hours, cooled to room temperature and water (10 ml) was added. The volatiles were removed under high vacuum and the residue was partitioned between saturated sodium bicarbonate (200 ml) and ethyl acetate (200 ml). The aqueous layer was back extracted with ethyl acetate (2×200 ml) and the combined extracts were washed with brine, dried over magnesium sulfate and filtered. After evaporation the product (8 g) was obtained.

¹H NMR (300 MHz, CDCl₃): δ: 7.32-7.13 (m, 5H), 3.53-3.41 (m, 1H), 3.50 (s, 2H), 3.20-3.17 (m, 2H), 2.02-1.94 (m, 2H), 1.76-1.68 (m, 4H), 1.56-1.41 (m, 2H)

### 8e) 8-Benzyl-8-aza-bicyclo[3.2.1]oct-3-ylamine

To a solution of azide (8d) (7.52 g) in THF (369 ml) and water (5.6 ml) was added triphenylphosphine (9.77 g). The reaction mixture was refluxed over night. After cooling, the volatiles were removed under reduced pressure and the residue was partitioned between 2N hydrochloric acid solution (200 ml) and ethyl acetate (200 ml). The aqueous layer washed three times with ethyl acetate (3×150 ml). The pH of the aqueous layer was adjusted to 14 by adding solid sodium hydroxide and then extracted with ethyl acetate (3×150 ml). The combined extracts were washed with brine, dried over magnesium sulfate and filtered. After evaporation, the residue was purified by flash chromatography (silica gel, dichloromethane/methanol 9:1+1% ammonia) to give the desired product (3.86 g).

¹H NMR (300 MHz, CDCl₃): δ: 7.42-7.22 (m, 5H), 3.59 (s, 2H), 3.24-3.22 (m, 2H), 3.09-2.98 (m, 1H), 2.54 (s, 2H), 2.09-1.97 (m, 2H), 1.78-1.68 (m, 2H), 1.66-1.53 (m, 4H)

### 8f) (8-Benzyl-8-aza-bicyclo[3.2.1]oct-3-yl)-carbamic acid tert-butyl ester

To a solution of amine (8e) (3.42 g) in 10% triethylamine in methanol (26 ml) was added Boc-anhydride (6.89 g) at room temperature. The mixture was stirred at room temperature over night, and the volatiles were removed under reduced pressure. The residue was taken up in dichloromethane, washed with water and brine, dried over magnesium sulfate, filtered and evaporated. The residue was purified by flash chromatography (silica gel, dichloromethane/(methanol/ammonia 9:1) 98:2, (dichloromethane/(methanol/ammonia 9:1) 19:1) to give the desired product (4.54 g).

¹H NMR (300 MHz, CDCl₃): δ: 7.33-7.15 (m, 5H), 4.36-4.24 (m, 1H), 3.84-3.66 (m, 1H), 3.49 (s, 2H), 3.21-3.10 (m, 2H), 2.04-1.90 (m, 2H), 1.80-1.71 (m, 2H), 1.70-1.59 (m, 2H), 1.57-1.42 (m, 2H), 1.36 (s, 9H)

### 8g) 8-Aza-bicyclo[3.2.1]oct-3-yl)-carbamic acid tert-butyl ester

To a solution of Boc-protected amine (8f) (4.76 g) in THF (75 ml) and methanol (75 ml) was added 20% Pd(OH)₂ (3.17 g). The reaction mixture was stirred at room temperature under a hydrogen atmosphere for 3 hours. The catalyst was filtered off and the filtrate evaporated. The residue was purified by flash chromatography (silica gel, dichloromethane/methanol 9:1+1% ammonia) to give the desired product (3.29 g).

¹H NMR (300 MHz, CDCl₃): δ: 4.57-4.45 (m, 1H), 3.91-3.75 (m, 1H), 3.61-3.52 (m, 2H), 2.93 (bs, 2H), 1.98-1.88 (m, 2H), 1.87-1.68 (m, 4H), 1.42-1.32 (m, 2H), 1.41 (s, 9H)

### 8h) {8-[2-Hydroxy-2-(6-methoxy-[1,5]-naphthyridin-4-yl)-ethyl]-8-aza-bicyclo[3.2.1]oct-3-yl}-carbamic acid tert-butyl ester (enantiomer 1)

2-Methoxy-8-oxiranyl-[1,5]-naphthyridine (1f) (726 mg) and amine (8 g) (813 mg) were dissolved in DMF (9 ml), treated with potassium carbonate (521 mg) and lithium perchlorate (382 mg) and stirred at 80° C. over night. The mixture was concentrated, dissolved in dichloromethane/methanol 9:1 and washed with water. The organic layer was dried over magnesium sulfate, filtered and concentrated. The residue was purified by flash chromatography (silica gel, dichloromethane/methanol 97:3) to give the desired product (1.28 g).

¹H NMR (300 MHz, d₆-DMSO): δ: 8.78 (d, 1H), 8.26 (d, 1H), 7.78 (d, 1H), 7.28 (d, 1H), 6.69 (bs, 1H), 5.52 (bs, 1H), 4.03 (s, 3H), 3.65-3.52 (m, 2H), 3.40-3.28 (m, 2H), 1.98-1.80 (m, 2H), 1.72-1.43 (m, 6H), 1.39-1.28 (m, 2H), 1.35 (s, 9H)

### 8i) 2-(3-Amino-8-aza-bicyclo[3.2.1]oct-8-yl)-1-(6-methoxy-[1,5]-naphthyridin-4-yl)-ethanol (enantiomer 1)

Compound (8h) (1.28 g) was dissolved in dichloromethane (23 ml), treated with TFA (2.3 ml) and stirred at room temperature over night. The mixture was made alkaline with 2N aqueous sodium hydroxide solution and the layers were separated. The aqueous layer was extracted with dichloromethane. The combined organic layers were washed with water and brine, dried over magnesium sulfate, filtered and concentrated. The residue was purified by flash chromatography (silica gel, dichloromethane/(methanol/ammonia 9:1) 9:1) to give the desired product (718 mg).

¹H NMR (300 MHz, d₆-DMSO): δ: 8.78 (d, 1H), 8.25 (d, 1H), 7.78 (d, 1H), 7.25 (d, 1H), 5.65-5.55 (m, 1H), 5.18 (bs, 1H), 4.02 (s, 3H), 3.39-3.28 (m, 1H), 3.24-3.14 (m, 1H), 2.96-2.86 (m, 1H), 2.84-2.68 (m, 1H), 2.40-2.28 (m, 1H), 1.90-1.69 (m, 2H), 1.62-1.43 (m, 5H), 1.41-1.22 (m, 3H)

### 8j) Title Compound

The compound was prepared as in example 1k from amine (8i) and aldehyde (1j).

¹H NMR (300 MHz, d₆-DMSO): δ: 10.78 (bs, 1H), 8.78 (d, 1H), 8.26 (d, 1H), 7.78 (d, 1H), 7.26 (d, 1H), 6.90 (s, 3H), 5.76 (s, 1H), 5.70-5.60 (m, 1H), 5.24 (bs, 1H), 4.55 (s, 2H), 4.02 (s, 3H), 3.72 (bs, 2H), 3.50-3.41 (m, 1H), 3.04-2.88 (m, 2H), 2.50-2.39 (m, 2H), 1.94-1.68 (m, 4H), 1.61-1.40 (m, 4H)

### Example 9

### 2-{3-[(Benzo[1,3]dioxol-5-ylmethyl)-amino]-8-aza-bicyclo[3.2.1]oct-8-yl}-1-(6-methoxy-[1,5]-naphthyridin-4-yl)-ethanol (enantiomer 1)

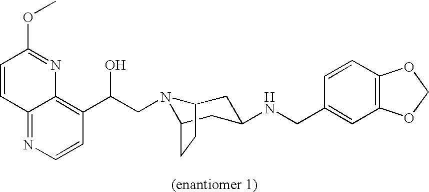

The compound was prepared as in example 1k from benzo[1,3]dioxole-5-carbaldehyde.

¹H NMR (300 MHz, d₆-DMSO): δ: 8.79 (d, 1H), 8.28 (d, 1H), 7.79 (d, 1H), 7.27 (d, 1H), 7.00 (s, 1H), 6.86 (s, 2H), 6.01 (s, 2H), 5.78 (s, 2H), 5.70-5.61 (m, 1H), 4.02 (s, 3H), 3.80 (bs, 2H), 3.50-3.44 (m, 1H), 3.18-2.91 (m, 2H), 2.50-2.42 (m, 1H), 1.92-1.70 (m, 4H), 1.68-1.40 (m, 4H)

### Example 10

### 2-{3-[(2,3-Dihydro-benzo[1,4]dioxin-6-ylmethyl)-amino]-8-aza-bicyclo[3.2.1]oct-8-yl}-1-(6-methoxy-[1,5]-naphthyridin-4-yl)-ethanol (enantiomer 1)

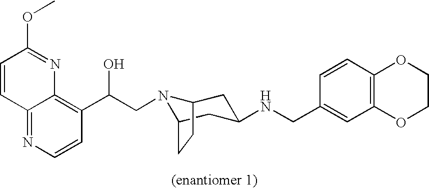

The compound was prepared as in example 1k from 2,3-dihydro-benzo[1,4]dioxine-6-carbaldehyde.

¹H NMR (300 MHz, d₆-DMSO): δ: 8.78 (d, 2H), 8.26 (d, 1H), 7.78 (d, 1H), 7.26 (d, 1H), 6.95 (s, 1H), 6.90-6.80 (m, 2H), 5.75 (s, 1H), 5.70-5.60 (m, 1H), 5.28 (bs, 1H), 4.22 (s, 4H), 4.01 (s, 3H), 3.79 (bs, 2H), 3.50-3.44 (m, 1H), 3.10-3.01 (bs, 1H), 3.00-2.90 (m, 1H), 1.95-1.72 (m, 4H), 1.70-1.54 (m, 2H), 1.52-1.42 (m, 2H), 1.38-1.22 (m, 2H)

### Example 11

### 2-{3-[(Benzo[1,2,5]thiadiazol-5-ylmethyl)-amino]-8-aza-bicyclo[3.2.1]oct-8-yl}-1-(6-methoxy-[1,5]-naphthyridin-4-yl)-ethanol (enantiomer 1)

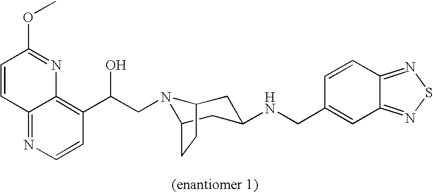

The compound was prepared as in example 1k from benzo[1,2,5]thiadiazole-5-carbaldehyde.

¹H NMR (300 MHz, d₆-DMSO): δ: 8.78 (d, 1H), 8.25 (d, 1H), 8.06-7.90 (m, 2H), 7.88-7.62 (m, 2H), 7.24 (d, 1H), 5.71-5.55 (m, 1H), 4.04 (s, 3H), 3.88 (s, 2H), 4.49-3.35 (m, 1H), 3.30-3.20 (m, 1H), 3.04-2.85 (m, 1H), 2.84-2.66 (m, 1H), 2.46-2.30 (m, 2H), 1.91-1.60 (m, 4H), 1.55-1.34 (m, 4H), 1.31-1.14 (m, 1H)

### Example 12

### 2-{3-[(Benzo[1,2,5]oxadiazol-5-ylmethyl)-amino]-8-aza-bicyclo[3.2.1]oct-8-yl}-1-(6-methoxy-[1,5]-naphthyridin-4-yl)-ethanol (enantiomer 1)

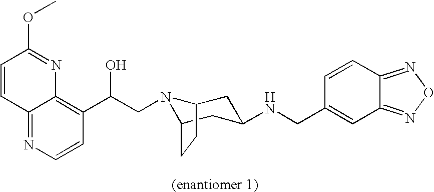

The compound was prepared as in example 1k from benzo[1,2,5]oxadiazole-5-carbaldehyde.

¹H NMR (300 MHz, d₆-DMSO): δ: 8.78 (d, 1H), 8.26 (d, 1H), 7.98 (d, 1H), 7.84 (s, 1H), 7.79 (d, 1H), 7.58 (d, 1H), 7.25 (d, 1H), 5.70-5.60 (m, 1H), 4.01 (s, 3H), 3.80 (s, 2H), 3.51-3.39 (m, 1H), 3.58-3.26 (bs, 3H), 3.04-2.90 (m, 1H), 2.85-2.68 (m, 1H), 2.50-2.38 (m, 1H), 1.92-1.66 (m, 4H), 1.58-1.35 (m, 4H)

### Example 13

### 6-({8-[2-Hydroxy-2-(6-methoxy-[1,5]-naphthyridin-4-yl)-ethyl]-8-aza-bicyclo[3.2.1]oct-3-ylamino}-methyl)-4H-benzo[1,4]thiazin-3-one (enantiomer 1)

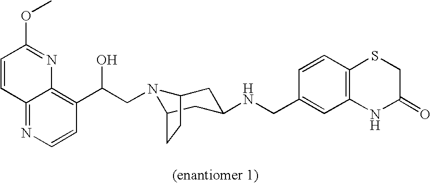

The compound was prepared as in example 1k from aldehyde (6b).

¹H NMR (300 MHz, d₆-DMSO): δ: 10.50 (s, 1H), 8.78 (d, 1H), 8.25 (d, 1H), 7.78 (d, 1H), 7.29-7.16 (m, 2H), 6.98-6.84 (m, 2H), 5.66-5.56 (m, 1H), 5.17 (bs, 1H), 4.18-4.06 (m, 1H), 4.00 (s, 3H), 3.59 (s, 2H), 3.41 (s, 2H), 3.40-3.28 (m, 2H), 2.98-2.86 (m, 1H), 2.76-2.61 (m, 1H), 2.43-2.30 (m, 1H), 1.87-1.59 (m, 4H), 1.55-1.28 (m, 4H)

### Example 14

### 1-(6-Methoxy-[1,5]-naphthyridin-4-yl)-2-[3-((E)-3-phenyl-allylamino)-8-aza-bicyclo[3.2.1]oct-8-yl]-ethanol (enantiomer 1)

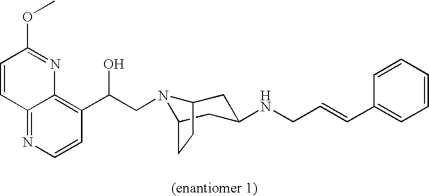

The compound was prepared as in example 1k from cinnamic aldehyde.

¹H NMR (300 MHz, d₆-DMSO): δ: 8.78 (d, 1H), 8.26 (d, 1H), 7.78 (d, 1H), 7.44-7.15 (m, 6H), 6.48 (d, 1H), 6.32-6.20 (m, 1H), 5.68-5.55 (m, 1H), 5.15 (bs, 1H), 4.01 (s, 3H), 3.44-3.15 (m, 5H), 2.98-2.86 (m, 1H), 2.80-2.64 (m, 1H), 2.45-2.30 (m, 1H), 1.92-1.60 (m, 4H), 1.55-1.21 (m, 4H)

### Example 15

### 3-{[(3-Oxo-3,4-dihydro-2H-benzo[1,4]oxazin-6-ylmethyl)-amino]-methyl}-cyclohexanecarboxylic acid (6-methoxy-[1,5]-naphthyridin-4-yl)-amide

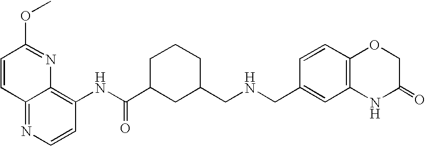

### 15a) [3-(6-Methoxy-[1,5]-naphthyridin-4-ylcarbamoyl)-cyclohexylmethyl]-carbamic acid tert-butyl ester

Triflate (1b) (22.56 g) and propylamine hydrochloride (41.97 g) were dissolved in pyridine (210 ml) and refluxed over night. The mixture was evaporated and the residue dissolved in water. The pH was adjusted to 12 with 1N sodium hydroxide solution. The aqueous layer was extracted twice with ethyl acetate. The combined organic layers were washed twice with water and once with brine, dried over magnesium sulfate, filtered and concentrated. The residue was purified by flash chromatography (silica gel, ethyl acetate, then ethyl acetate/methanol 9:1) to give the desired product (12.28 g).

¹H NMR (300 MHz, CDCl₃): δ: 8.30 (d, 1H), 8.02 (d, 1H), 7.00 (d, 1H), 6.64 (d, 1H), 5.27 (bs, 2H), 3.98 (s, 3H)

### 15b) 3-Aminomethyl-cyclohexanecarboxylic acid (6-methoxy-[1,5]-naphthyridin-4-yl)-amide

Quinoline amine (15a) (1.93 g) and 3-(tert-Butoxycarbonylaminomethyl)-cyclohexanecarboxylic acid (Prepared according to the method of Yang, J. Med. Chem., 1998, p. 2175-2179) (2.84 g) were suspended in DMF (60 ml), then HATU (4.2 g) and triethylamine (3.1 ml) were added. The mixture was heated at 60° C. over night. The solvent was evaporated and the residue partitioned between ethyl acetate and brine. The organic layer was dried over magnesium sulfate, filtered and evaporated. The residue was recrystallised from ethyl acetate and pentane to give the desired product (2.24 g).

¹H NMR (300 MHz, d₆-DMSO): δ: 9.75 (s, 1H), 8.67 (d, 1H), 8.40 (d, 1H), 8.26 (d, 1H), 7.30 (d, 1H), 6.91-6.88 (m, 1H), 4.14 (s, 3H), 3.14-3.02 (m, 1H), 2.93-2.68 (m, 3H), 2.08-1.92 (m, 2H), 1.89-1.78 (m, 1H), 1.74-1.64 (m, 1H), 1.60-1.45 (m, 1H), 1.37 (s, 9H), 1.22-1.03 (m, 2H), 0.95-0.78 (m, 1H)

### 15c) 3-Aminomethyl-cyclohexanecarboxylic acid (6-methoxy-[1,5]-naphthyridin-4-yl)-amide

Naphthyridine amide (15b) (2.24 g) was dissolved in dichloromethane (128 ml), treated with 3A sieves (3.40 g) and boron trifluoride etherate (3.4 ml) at 0° C. and stirred at this temperature for 15 minutes, then at room temperature over night. The sieves were filtered off and washed with ethyl acetate, dichloromethane and methanol. The filtrate was evaporated and the residue was purified by flash chromatography (silica gel, dichloromethane/methanol 9:1+1% ammonia) to give the desired product (1.56 g).

¹H NMR (300 MHz, d₆-DMSO): δ: 9.77 (s, 1H), 8.67 (d, 1H), 8.40 (d, 1H), 8.26 (d, 1H), 7.31 (d, 1H), 4.14 (s, 3H), 2.80-2.71 (m, 1H), 2.62-2.58 (m, 2H), 2.13-1.95 (m, 2H), 1.90-1.72 (m, 2H), 1.64-1.46 (m, 1H), 1.44-1.30 (m, 2H), 1.25-1.07 (m, 1H), 1.00-0.82 (m, 1H)

### 15d) Title Compound

The compound was prepared as in example 1k from amine (15c) and aldehyde (1j).

¹H NMR (300 MHz, d₆-DMSO): δ: 10.49 (s, 1H), 9.53 (s, 1H), 8.44 (d, 1H), 8.16 (d, 1H), 8.03 (d, 1H), 7.07 (d, 1H), 6.69-6.63 (m, 3H), 6.46 (bs, 1H), 4.30 (s, 2H), 3.88 (s, 3H), 3.11 (bs, 2H), 2.53-2.46 (m, 1H), 2.27-2.17 (m, 2H), 1.91-1.87 (m, 1H), 1.78-1.76 (m, 1H), 1.62-1.57 (m, 1H), 1.49-1.29 (m, 1H), 1.24-1.06 (m, 2H), 1.02-0.81 (m, 2H), 0.72-0.60 (m, 1H)

### Example 16

### 3-{[(3-Oxo-3,4-dihydro-2H-benzo[1,4]thiazin-6-ylmethyl)-amino]-methyl}-cyclohexanecarboxylic acid (6-methoxy-[1,5]-naphthyridin-4-yl)-amide

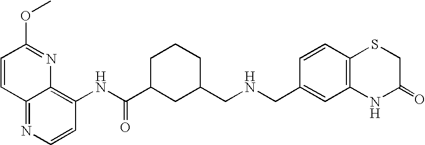

The compound was prepared as in example 1k from aldehyde (6b).

¹H NMR (300 MHz, d₆-DMSO): δ: 10.54 (s, 1H), 9.76 (s, 1H), 8.67 (d, 1H), 8.40 (d, 1H), 8.26 (d, 1H), 7.32-7.23 (m, 2H), 6.97-6.94 (m, 2H), 4.11 (s, 3H), 3.66 (s, 2H), 3.43 (s, 2H), 2.76-2.68 (m, 1H), 2.44-2.36 (m, 1H), 2.14-2.10 (m, 1H), 2.05-1.94 (m, 1H), 1.89-1.78 (m, 2H), 1.68-1.50 (m, 1H), 1.46-1.30 (m, 2H), 1.25-1.05 (m, 2H), 0.98-0.82 (m, 1H)

### Example 17

### 3-{[(3-Oxo-3,4-dihydro-2H-pyrido[3,2-b][1,4]thiazin-6-ylmethyl)-amino]-methyl}-cyclohexanecarboxylic acid (6-methoxy-[1,5]-naphthyridin-4-yl)-amide

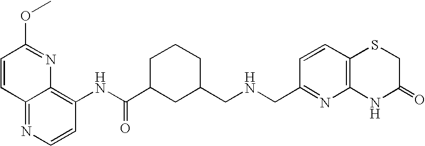

### 17a) N-(6-Methyl-pyridin-2-yl)-acetamide

A solution of 3-amino-6-picoline (39 g) in acetic anhydride (200 ml) was heated at 70° C. for 90 minutes. The volatiles were removed under reduced pressure, the residue was taken up in water (500 ml) and sodium bicarbonate was added until pH 8 was reached. The solid formed was extracted with ethyl acetate (2×200 ml). The combined extracts were washed with brine, dried over sodium sulfate, filtered and evaporated to give the desired product (53.3 g).

¹H NMR (300 MHz, CDCl₃): δ: 8.43 (bs, 1H), 8.00 (d, 1H), 7.62-7.57 (m, 1H), 6.89 (d, 1H), 2.45 (s, 3H), 2.18 (s, 3H)

### 17b) 6-Acetylamino-pyridine-2-carboxylic acid

A solution of acetamide (17a) (53.3 g) in water (530 ml) was heated at 75° C. until a homogenous solution was formed. Potassium permanganate (133 g) was then added in small portions over 1.25 hours (the reaction temperature was carefully monitored with an internal thermometer). After stirring for 3 hours at 75° C. the reaction mixture was filtered through Celite® while still hot. The filter cake washed with hot water. The filtrate was concentrated to about 100 ml. Concentrated hydrochloric acid was added until a white solid formed. The white solid was collected by filtration and dried under vacuum to give the desired product (32 g).

¹H NMR (300 MHz, d₆-DMSO): δ: 10.85 (s, 1H), 8.26 (d, 1H), 7.97-7.72 (m, 1H), 7.73 (dd, 1H), 2.11 (s, 3H)

### 17c) 6-Amino-pyridine-2-carboxylic acid methyl ester

Acid (17b) (18 g) was suspended in methanol saturated with gaseous hydrochloric acid. The mixture was refluxed overnight and after cooling, concentrated to dryness. The residue was partitioned between water and dichloromethane. Solid sodium bicarbonate was added and the layers were separated. The aqueous layer was back extracted with dichloromethane (200 ml). The combined organic layers were washed with brine, dried over sodium sulfate, filtered and evaporated. The residue was purified by flash chromatography (silica gel, dichloromethane/ethyl acetate 1:1) to give the desired product (9.64 g).

¹H NMR (300 MHz, CDCl₃): δ: 7.52-7.41 (m, 2H), 6.66 (dd, 1H), 5.12 (bs, 2H), 3.91 (s, 3H)

### 17d) 6-Amino-5-bromo-pyridine-2-carboxylic acid methyl ester

To a solution of ester (17c) (9.64 g) in chloroform (408 ml) was added a solution of bromine (3.35 ml) in chloroform (70 ml) over 1 hour. After stirring at room temperature for 40 hours, saturated aqueous sodium thiosulfate (150 ml) was added and the organic layer was separated. The aqueous layer was extracted once with dichloromethane. The combined organic layers were washed with brine, dried over sodium sulfate, filtered and evaporated. The residue was purified by flash chromatography (silica gel, hexane/ethyl acetate 2:1) to give the desired product (1.8 g).

¹H NMR (300 MHz, CDCl₃): δ: 7.73 (d, 1H), 7.29 (d, 1H), 5.39 (bs, 2H), 3.90 (s, 3H)

### 17e) 3-Oxo-3,4-dihydro-2H-pyrido[3,2-b][1,4]thiazine-6-carboxylic acid methyl ester

To a solution of methyl thioglycolate (2.4 ml) in DMF (75 ml) was added sodium hydride (1.1 g). After 1 hour, the bromopyridine (17d) (5.0 g) was added and the reaction mixture stirred at room temperature for 12 hours and then diluted with water (150 ml). The precipitate was filtered off and scarcely washed with ethyl acetate and acetonitrile to give the desired product (1.65 g).

¹H NMR (300 MHz, d₆-DMSO): δ: 11.29 (s, 1H), 7.97 (d, 1H), 7.66 (d, 1H), 3.86 (s, 3H), 3.64 (s, 2H), 3.33 (s, 3H).

### 17f) 3-Oxo-3,4-dihydro-2H-pyrido[3,2-b][1,4]thiazine-6-carboxylic acid

To a solution of ester (17e) (2.33 g) in dioxane (354 ml) and water (90 ml) was added dropwise over 2 hours 0.5N sodium hydroxide solution (24 ml). The reaction mixture was stirred at room temperature overnight. The solvent was removed under reduced pressure, the residue diluted with water (10 ml) and adjusted to pH 4 by adding 2N hydrochloric acid solution. The resulting white solid was filtered off, washed sparsely with water and dried overnight under vacuum to give the desired product (1.72 g).

MS (EI): m/z: 211 [M+H]⁺

### 17g) 6-Hydroxymethyl-4H-pyrido[3,2-b][1,4]thiazin-3-one

To a solution of acid (17f) (1.72 g) in THF (82 ml) cooled to −10° C., was added triethylamine (1.4 ml) and then isobutyl chloroformate (1.2 ml). After 25 minutes, the resulting heterogeneous mixture was filtered though a pad of Celite® into an ice cooled solution of sodium borohydride (1.1 g) in water (28 ml). The resulting mixture was stirred at the same temperature for 30 minutes and then 0.2N hydrochloric acid solution was added to adjust the pH to 7. After evaporation, the solid was filtered off, washed with water and dried under vacuum to give the desired product (1.1 g).

MS (EI): m/z: 197 [M+H]⁺

### 17h) 3-Oxo-3,4-dihydro-2H-pyrido[3,2-b][1,4]thiazine-6-carbaldehyde

To a solution of alcohol (17g) (1.1 g) in dichloromethane (100 ml) and THF (100 ml) was added manganese dioxide (2.5 g). After stirring at room temperature for 90 minutes, more manganese dioxide (3 g) was added and the mixture was stirred at room temperature for a further 2 hours. The reaction mixture was then filtered through a plug of Celite® and the filtrate concentrated to give the desired product (598 mg).

¹H NMR (300 MHz, CDCl₃): δ: 9.85 (s, 1H), 8.40 (bs, 1H), 7.74 (d, 1H), 7.55 (d, 1H), 3.50 (s, 2H)

### 17i) Title Compound

The compound was prepared as in example 1k from aldehyde (17h).

¹H NMR (300 MHz, d₆-DMSO): δ: 10.87 (s, 1H), 9.77 (s, 1H), 8.67 (d, 1H), 8.40 (d, 1H), 8.27 (d, 1H), 7.73 (d, 1H), 7.31 (d, 1H), 7.08 (d, 1H), 4.11 (s, 3H), 3.69 (s, 2H), 3.52 (s, 2H), 2.77-2.69 (m, 1H), 2.46-2.33 (m, 2H), 2.20-2.10 (m, 1H), 2.07-1.90 (m, 1H), 1.86-1.72 (m, 2H), 1.68-1.50 (m, 1H), 1.45-1.29 (m, 2H), 1.15-1.06 (m, 2H), 1.00-0.81 (m, 1H)

### Example 18

### 3-{[(Benzo[1,3]dioxol-5-ylmethyl)-amino]-methyl}-cyclohexanecarboxylic acid (6-methoxy-[1,5]-naphthyridin-4-yl)-amide

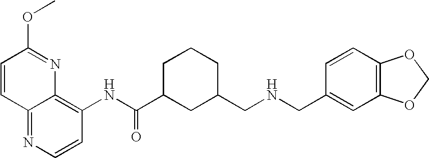

The compound was prepared as in example 1k from benzo[1,3]dioxole-5-carbaldehyde.

¹H NMR (300 MHz, d₆-DMSO): δ: 9.76 (s, 1H), 8.67 (d, 1H), 8.39 (d, 1H), 8.27 (d, 1H), 7.31 (d, 1H), 6.93 (s, 1H), 6.85-6.77 (m, 2H), 5.97 (s, 2H), 4.11 (s, 3H), 3.64 (s, 2H), 2.76-2.68 (m, 1H), 2.46-2.34 (m, 2H), 2.19-2.09 (m, 1H), 2.06-1.95 (m, 1H), 1.88-1.71 (m, 2H), 1.68-1.50 (m, 1H), 1.46-1.24 (m, 2H), 1.23-1.02 (m, 2H), 0.95-0.90 (m, 1H)

### Example 19

### 3-{[(2,3-Dihydro-benzo[1,4]dioxin-6-ylmethyl)-amino]-methyl}-cyclohexanecarboxylic acid (6-methoxy-[1,5]-naphthyridin-4-yl)-amide

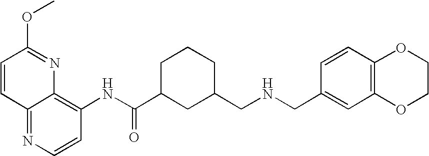

The compound was prepared as in example 1k from 2,3-dihydro-benzo[1,4]dioxine-6-carbaldehyde.

¹H NMR (300 MHz, d₆-DMSO): δ: 9.42 (s, 1H), 8.58 (d, 1H), 8.37 (d, 1H), 8.09 (d, 1H), 7.06 (d, 1H), 6.80-6.71 (m, 3H), 4.15 (s, 3H), 4.02 (s, 2H), 3.73 (s, 1H), 2.61-2.51 (m, 1H), 2.48-2.35 (m, 1H), 2.22-2.11 (m, 1H), 2.08-1.98 (m, 1H), 1.89-1.62 (m, 3H), 1.49-1.27 (m, 3H), 1.26-1.12 (m, 4H), 0.99-0.72 (m, 2H)

### Example 20

### 3-{[(Benzo[1,2,5]thiadiazol-5-ylmethyl)-amino]-methyl}-cyclohexanecarboxylic acid (6-methoxy-[1,5]-naphthyridin-4-yl)-amide

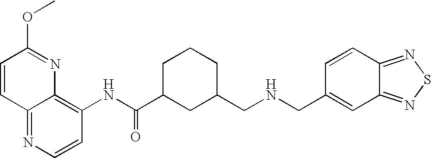

The compound was prepared as in example 1k from benzo[1,2,5]thiadiazole-5-carbaldehyde.

¹H NMR (300 MHz, d₆-DMSO): δ: 9.74 (s, 1H), 8.65 (d, 1H), 8.39 (d, 1H), 8.25 (d, 1H), 8.02-7.95 (m, 2H), 7.71 (dd, 1H), 7.28 (d, 1H), 4.08 (s, 3H), 3.88 (s, 2H), 2.76-2.68 (m, 1H), 2.46-2.37 (m, 2H), 2.24-2.10 (m, 1H), 2.06-1.95 (m, 1H), 1.89-1.74 (m, 2H), 1.70-1.51 (m, 1H), 1.49-1.30 (m, 2H), 1.25-1.05 (m, 2H), 0.99-0.81 (m, 1H)

### Example 21

### 3-[((E)-3-Pyridin-2-yl-allylamino)-methyl]-cyclohexanecarboxylic acid (6-methoxy-[1,5]-naphthyridin-4-yl)-amide

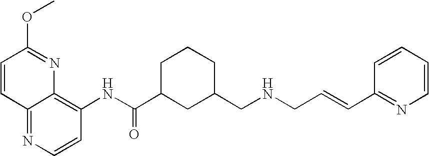

The compound was prepared as in example 1k from aldehyde (5a).

¹H NMR (300 MHz, d₆-DMSO): δ: 9.77 (s, 1H), 8.67 (d, 1H), 8.51-8.48 (m, 1H), 8.40 (d, 1H), 8.26 (d, 1H), 7.75-7.69 (m, 1H), 7.40 (d, 1H), 7.30 (d, 1H), 7.22-7.18 (m, 1H), 6.80-6.71 (m, 1H), 6.58 (d, 1H), 5.76 (s, 1H), 4.12 (s, 3H), 3.36-3.34 (m, 1H), 2.77-2.69 (m, 1H), 2.48-2.39 (m, 2H), 2.22-2.10 (m, 1H), 2.05-1.95 (m, 1H), 1.89-1.72 (m, 2H), 1.65-1.50 (m, 1H), 1.48-1.31 (m, 2H), 1.24-1.07 (m, 2H), 1.00-0.82 (m, 1H)

### Example 22

### 3-[((E)-3-Phenyl-allylamino)-methyl]-cyclohexanecarboxylic acid (6-methoxy-[1,5]-naphthyridin-4-yl)-amide

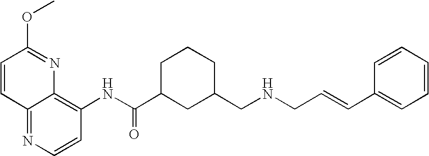

The compound was prepared as in example 1k from cinnamic aldehyde.

¹H NMR (300 MHz, d₆-DMSO): δ: 9.77 (s, 1H), 8.67 (d, 1H), 8.40 (d, 1H), 8.26 (d, 1H), 7.42-7.36 (m, 2H), 7.33-7.24 (m, 3H), 7.24-7.19 (m, 1H), 6.55-6.50 (m, 1H), 6.35-6.26 (m, 1H), 4.12 (s, 3H), 3.20-3.13 (m, 1H), 2.80-2.64 (m, 1H), 2.50-2.38 (m, 2H), 2.20-2.10 (m, 1H), 2.06-1.92 (m, 1H), 1.88-1.72 (m, 2H), 1.65-1.50 (m, 1H), 1.47-1.04 (m, 5H), 0.98-0.81 (m, 1H)

### Example 23

### 3-{[(Benzo[1,2,5]oxadiazol-5-ylmethyl)-amino]-methyl}-cyclohexanecarboxylic acid (6-methoxy-[1,5]-naphthyridin-4-yl)-amide

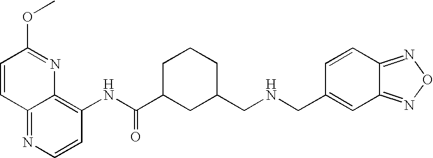

The compound was prepared as in example 1k from benzo[1,2,5]oxadiazole-5-carbaldehyde.

¹H NMR (300 MHz, d₆-DMSO): δ: 9.75 (s, 1H), 8.66 (d, 1H), 8.39 (d, 1H), 8.25 (d, 1H), 8.04-7.94 (m, 1H), 7.86-7.79 (m, 1H), 7.59 (d, 1H), 7.29 (d, 1H), 4.10 (s, 3H), 3.81 (s, 2H), 2.76-2.69 (m, 1H), 2.46-2.37 (m, 2H), 2.22-2.09 (m, 1H), 2.05-1.94 (m, 1H), 1.90-1.75 (m, 2H), 1.70-1.52 (m, 1H), 1.48-1.30 (m, 2H), 1.24-1.06 (m, 1H), 1.00-0.80 (m, 1H)

### Example 24

### 3-{[(7-Fluoro-3-oxo-3,4-dihydro-2H-benzo[1,4]thiazin-6-ylmethyl)-amino]-methyl}-cyclohexanecarboxylic acid (6-methoxy-[1,5]-naphthyridin-4-yl)-amide

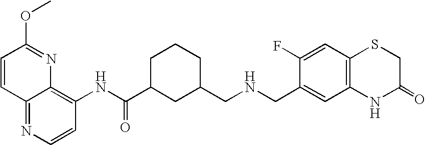

### 24a) 2,4-Difluoro-benzoic acid ethyl ester

2,4-Difluorobenzoic acid (5.00 g) was dissolved in ethanol (50 ml). Then gaseous hydrogen chloride was bubbled through the solution for 20 minutes. The mixture was refluxed for 5 hours, then concentrated and the residue dissolved in diethyl ether. The organic layer washed with 1N sodium hydroxide solution and brine, dried over magnesium sulfate, filtered and evaporated to give the desired product (3.8 g).

¹H NMR (300 MHz, CDCl₃): δ: 8.05-7.95 (m, 1H), 6.99-6.82 (m, 2H), 4.40 (q, 2H), 1.22 (t, 3H)

### 24b) 2,4-Difluoro-5-nitro-benzoic acid ethyl ester

Ethyl ester (24a) (3.8 g) was dissolved in fuming nitric acid (3 ml) and concentrated sulfuric acid (3 ml) at 0° C. and stirred for 2.5 hours. The mixture was diluted with water (10 ml) and extracted with dichloromethane (200 ml). The organic layer was washed with brine, dried over magnesium sulfate, filtered and evaporated. The residue was purified by flash chromatography (silica gel, ethyl acetate/hexane 1:6) to give the desired product (3.96 g).

¹H NMR (300 MHz, CDCl₃): δ: 8.70 (m, 1H), 7.05 (m, 1H), 4.36 (q, 2H), 1.35 (t, 3H)

### 24c) 2-Fluoro-4-methoxycarbonylmethylsulfanyl-5-nitro-benzoic acid ethyl ester

Nitrobenzoic acid (24b) (3.96 g) was dissolved in dichloromethane (75 ml), treated with triethylamine (2.8 ml) and cooled to 0° C. After the addition of methyl thioglycolate (1.5 ml), the mixture was stirred at 0-5° C. for 3.5 hours and kept over night in the refrigerator. The mixture was concentrated and the residue purified by flash chromatography (silica gel, ethyl acetate/hexane 2:8) to give the desired product (3.86 g).

¹H NMR (300 MHz, CDCl₃): δ: 8.82 (d, 1H), 7.19 (d, 1H), 4.35 (q, 2H), 3.72 (s, 3H), 3.70 (s, 2H), 1.35 (t, 3H)

### 24d) 7-Fluoro-3-oxo-3,4-dihydro-2H-benzo[1,4]thiazine-6-carboxylic acid ethyl ester

Compound (24c) (3.86 g) was dissolved in acetic acid (142 ml), treated with iron powder (6.8 g) and stirred at 60° C. for 4 hours. The mixture was filtered through a pad of silica gel, washed with methanol and the filtrate was partially evaporated. Water and ethyl acetate were added and the layers separated. The aqueous layer was extracted once with ethyl acetate. The combined organic layers were washed four times with water, dried over magnesium sulfate, filtered and concentrated to give the desired product (3.11 g).

¹H NMR (300 MHz, d₆-DMSO): δ: 10.71 (s, 1H), 7.50-7.39 (m, 2H), 4.30 (q, 2H), 3.56 (s, 2H), 1.30 (t, 3H)

### 24e) 7-Fluoro-3-oxo-3,4-dihydro-2H-benzo[1,4]thiazine-6-carboxylic acid

Thiazine (24d) (3.11 g) was suspended in THF (37 ml), treated with 1N sodium hydroxide (37 ml) and stirred at room temperature over night. The mixture was acidified with 1N hydrochloric acid solution to pH 3 and partially evaporated. The precipitated solid was filtered off and washed with water. The solid was dried under reduced pressure (100 mbar, 40° C.) to give the desired product (2.49 g).

¹H NMR (300 MHz, d₆-DMSO): δ: 13.26 (bs, 1H), 10.72 (s, 1H), 7.48 (d, 1H), 7.38 (d, 1H), 3.57 (s, 2H)

### 24f) 7-Fluoro-6-hydroxymethyl-4H-benzo[1,4]thiazin-3-one

Thiazine acid (24e) (2.49 g) was suspended in dry THF (80 ml), cooled to 0° C., treated with triethylamine (1.8 ml) and isobutyl chloroformate (1.6 ml). The mixture was stirred at this temperature for 30 minutes. The mixture was quickly filtered through Celite® into a vigorously stirred solution of sodium borohydride (1.24 g) in ice water (24 ml). The mixture was stirred for a further 45 minutes. Then the suspension was acidified with 1N hydrochloric acid solution to pH 1 and extracted with ethyl acetate. The organic layer washed with brine, dried over magnesium sulfate, filtered and concentrated to give the desired product (2.29 g).

¹H NMR (300 MHz, d₆-DMSO): δ: 10.61 (s, 1H), 7.19 (d, 1H), 7.10 (d, 1H), 5.33 (m, 1H), 4.47 (d, 2H), 3.26 (s, 2H)

### 24g) 7-Fluoro-3-oxo-3,4-dihydro-2H-benzo[1,4]thiazine-6-carbaldehyde

Thiazinone (24f) (1.63 g) was dissolved in dichloromethane/THF 1:1 (138 ml), treated with manganese dioxide (6.63 g) and stirred at room temperature for 2 days. Then more manganese dioxide (3.32 g) was added and stirred for a further 3 days. The mixture was filtered through Celite®, washed with THF and the filtrate was evaporated. The residue was purified by flash chromatography (silica gel, ethyl acetate/hexane 3:7) to give the desired product (765 mg).

¹H NMR (300 MHz, d₆-DMSO): δ: 10.80 (s, 1H), 10.14 (s, 1H), 7.51 (d, 1H), 7.35 (d, 1H), 3.60 (s, 2H)

### 24h) Title Compound

The compound was prepared as in example 1k from aldehyde (24g).

¹H NMR (300 MHz, d₆-DMSO): δ: 10.35 (s, 1H), 9.54 (s, 1H), 8.44 (d, 1H), 8.17 (d, 1H), 8.03 (d, 1H), 7.08 (d, 1H), 6.98 (d, 1H), 6.85 (d, 1H), 3.89 (s, 3H), 3.46 (s, 2H), 3.22 (s, 2H), 2.52-2.46 (m, 1H), 2.27-2.16 (m, 2H), 1.96-1.86 (m, 1H), 1.84-1.72 (m, 1H), 1.66-1.50 (m, 2H), 1.48-1.30 (m, 1H), 1.26-1.08 (m, 2H), 1.04-0.84 (m, 2H), 0.78-0.58 (m, 1H)

### Example 25

### 1-(3-Chloro-6-methoxy-[1,5]-naphthyridin-4-yl)-2-{3-[(2,3-dihydro-benzo[1,4]dioxin-6-ylmethyl)-amino]-8-aza-bicyclo[3.2.1]oct-8-yl}-ethanol

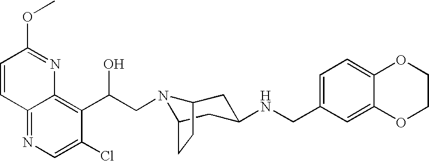

### 25a) 3-Chloro-6-methoxy-[1,5]-naphthyridin-4-ol

6-Methoxy-[1,5]-naphthyridin-4-ol (1a) (12 g) was suspended in acetic acid (200 ml) and warmed until all had dissolved, then NCS (10 g) was added and the mixture stirred at 35° C. over night. The mixture was cooled, the solid collected by filtration, washed with acetic acid and dried under vacuum to give the desired product (13.1 g).

¹H NMR (300 MHz, d₆-DMSO): δ: 12.30 (bs, 1H), 8.40 (s, 1H), 7.98 (d, 1H), 7.20 (d, 1H), 3.95 (s, 3H)

### 25b) Trifluoro-methanesulfonic acid 3-chloro-6-methoxy-[1,5]-naphthyridin-4-yl ester

Sodium hydride (80 mg) washed with hexane. The hexane was decanted and dry DMF (10 ml) added, followed by chloro-naphthyridine (25a) (4.5 g). The mixture was stirred at room temperature for 1 hour, then cooled with an ice bath, N-phenyltrifluoromethanesulphonimide (8.39 g) was added and the mixture was stirred at room temperature over night. The solvent was evaporated, then with toluene (30 ml) co-evaporated and then diluted with diethyl ether/dichloromethane 1:1. The organic layer washed with saturated sodium bicarbonate solution, dried over magnesium sulfate, filtered and evaporated. The residue was purified by flash chromatography (silica gel, dichloromethane) to give the desired product (4.75 g).

¹H NMR (300 MHz, CDCl₃): δ: 8.83 (s, 1H), 8.26 (d, 1H), 7.24 (d, 1H), 4.18 (s, 3H)

### 25c) 8-(1-Butoxy-vinyl)-7-chloro-2-methoxy-[1,5]-naphthyridine

Triflate (25b) (4.71 g) was dissolved in DMF (50 ml), then triethylamine (3.8 ml), n-Butylvinylether (11 ml), palladium (II) acetate (309 mg) and 1,3-bis(diphenylphosphino)propane (680 mg) were added. The mixture was stirred at 60-70° C. for 30 hours. The mixture was evaporated, then co-evaporated with toluene and purified by flash chromatography (silica gel, dichloromethane/hexane 1:1) to give the desired product (3.25 g).

¹H NMR (300 MHz, CDCl₃): δ: 8.68 (s, 1H), 8.14 (d, 1H), 7.04 (d, 1H), 4.64 (d, 1H), 4.24 (d, 1H), 3.97 (s, 3H), 3.92 (t, 2H), 1.73-1.64 (m, 2H), 1.46-1.34 (m, 2H), 0.88 (t, 3H)

### 25d) 2-Bromo-1-(3-chloro-6-methoxy-[1,5]-naphthyridin-4-yl)-ethanone

Vinylether (25c) (3.2 g) was dissolved in THF (49 ml), then water (4.4 ml) and N-bromoscuccinimide (3.2 g) were added and the mixture stirred for 5 hours at room temperature. The solvent was evaporated and the residue purified by flash chromatography (silica gel, dichloromethane/hexane 2:1) to give the desired product (2.13 g).

¹H NMR (300 MHz, CDCl₃): δ: 8.70 (s, 1H), 8.16 (d, 1H), 7.10 (d, 1H), 4.54 (s, 2H), 3.96 (s, 3H)

### 25e) 7-Chloro-2-methoxy-8-oxiranyl-[1,5]-naphthyridine

Bromoketone (25d) (1 g) was dissolved in methanol (15 ml) and water (3.8 ml). The mixture was cooled with an ice bath and sodium borohydride (247 mg) was added. The mixture was stirred for 1.5 hours at room temperature and then diluted with water and extracted three times with chloroform. The combined organic layers were dried over magnesium sulfate, filtered and evaporated. The intermediate was dissolved in methanol (4.8 ml), treated with potassium carbonate (483 mg) and stirred at room temperature for 3 hours. The mixture was diluted with water and extracted with chloroform, dried over magnesium sulfate, filtered and evaporated. The residue was purified by flash chromatography (silica gel, dichloromethane, dichloromethane/methanol 98:2) and recrystallised from diethyl ether/hexane to give the desired product (290 mg).

¹H NMR (300 MHz, CDCl₃): δ: 8.62 (s, 1H), 8.12 (d, 1H), 7.06 (d, 1H), 4.46 (m, 1H), 4.02 (s, 3H), 3.40 (m, 1H), 3.31 (m, 1H)

### 25f) {8-[2-(3-Chloro-6-methoxy-[1,5]-naphthyridin-4-yl)-2-hydroxy-ethyl]-8-aza-bicyclo[3.2.1]oct-3-yl}-carbamic acid tert-butyl ester

Epoxide (25e) (200 mg) and amine (8g) (96 mg) were dissolved in DMF (2 ml), treated with potassium carbonate (61 mg) and lithium perchlorate (45 mg) and heated in the microwave for 40 minutes at 130° C. The mixture was concentrated, dissolved in dichloromethane/methanol 9:1 and washed with water and brine. The organic layer was dried over magnesium sulfate, filtered and evaporated. The residue was purified by flash chromatography (silica gel, dichloromethane, dichloromethane/methanol 9:1+1% ammonia) to give the desired product (200 mg).

MS (EI): m/z: 463 [M+H]⁺

### 25g) 2-(3-Amino-8-aza-bicyclo[3.2.1]oct-8-yl)-1-(3-chloro-6-methoxy-[1,5]-naphthyridin-4-yl)-ethanol

Boc-amine (25f) (200 mg) was dissolved in dichloromethane (4 ml), treated with trifluoroacetic acid (0.33 ml) and stirred at room temperature over night. The mixture was made alkaline with 2N sodium hydroxide solution and the layers were separated. The aqueous layer was extracted once with dichloromethane. The combined organic layers were washed with brine, dried over magnesium sulfate, filtered and concentrated. The residue was purified by flash chromatography (silica gel, dichloromethane/methanol 9:1+1% ammonia) to give the desired product (97 mg).

¹H NMR (300 MHz, d₆-DMSO): δ: 8.34 (s, 1H), 8.08 (d, 1H), 7.09 (d, 1H), 5.61-5.49 (m, 1H), 5.42-5.28 (m, 1H), 3.83 (s, 3H), 3.19-2.97 (m, 4H), 2.87-2.66 (m, 3H), 2.58-2.43 (m, 1H), 1.68-1.44 (m, 2H), 1.30-1.12 (m, 4H), 0.98-0.82 (m, 1H)

### 25h) Title Compound

The compound was prepared as in example 1k from 2,3-dihydro-benzo[1,4]dioxine-6-carbaldehyde.

¹H NMR (300 MHz, d₆-DMSO): δ: 8.76 (s, 1H), 8.32 (d, 1H), 7.33 (d, 1H), 6.87-6.71 (m, 3H), 5.84-5.70 (m, 1H), 5.64-5.50 (m, 1H), 4.21 (s, 4H), 4.05 (s, 3H), 3.71-3.55 (m, 2H), 3.20-3.12 (m, 1H), 3.10-2.94 (m, 2H), 2.90-2.71 (m, 1H), 1.92-1.56 (m, 4H), 1.48-1.19 (m, 4H)

### Example 26

### 2-{3-[(Benzo[1,2,5]thiadiazol-5-ylmethyl)-amino]-8-aza-bicyclo[3.2.1]oct-8-yl}-1-(3-chloro-6-methoxy-[1,5]-naphthyridin-4-yl)-ethanol

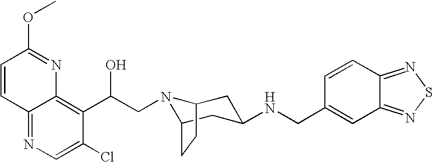

The compound was prepared as in example 1k from benzo[1,2,5]thiadiazole-5-carbaldehyde.

¹H NMR (300 MHz, d₆-DMSO): δ: 8.76 (s, 1H), 8.32 (d, 1H), 7.98 (d, 1H), 7.91 (s, 1H), 7.67 (dd, 1H), 7.32 (d, 1H), 5.81-5.74 (m, 1H), 5.59-5.56 (m, 1H), 4.03 (s, 3H), 3.81 (s, 2H), 3.13-2.92 (m, 3H), 2.78-2.59 (m, 1H), 1.85-1.55 (m, 4H), 1.45-1.13 (m, 4H)

### Example 27

### 6-({8-[2-(3-Chloro-6-methoxy-[1,5]-naphthyridin-4-yl)-2-hydroxy-ethyl]-8-aza-bicyclo[3.2.1]oct-3-ylamino}-methyl)-4H-benzo[1,4]oxazin-3-one

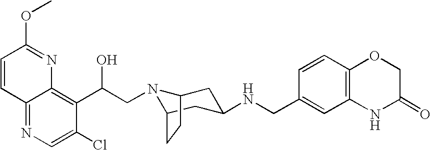

The compound was prepared as in example 1k from aldehyde (1j).

¹H NMR (300 MHz, d₆-DMSO): δ: 10.74 (s, 1H), 8.77 (s, 1H), 8.32 (d, 1H), 7.32 (d, 1H), 6.89 (s, 3H), 5.86-5.74 (m, 1H), 5.68-5.52 (m, 1H), 4.54 (s, 2H), 4.04 (s, 3H), 3.73 (s, 2H), 3.44-3.33 (m, 1H), 3.24-3.13 (m, 1H), 3.10-2.80 (m, 3H), 1.95-1.58 (m, 4H), 1.54-1.10 (m, 5H)

### Example 28

### 6-({8-[2-(3-Chloro-6-methoxy-[1,5]-naphthyridin-4-yl)-2-hydroxy-ethyl]-8-aza-bicyclo[3.2.1]oct-3-ylamino}-methyl)-7-fluoro-4H-benzo[1,4]thiazin-3-one

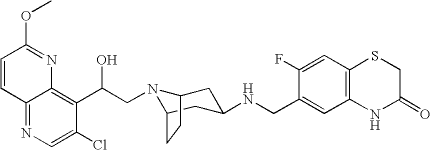

The compound was prepared as in example 1k from aldehyde (24g).

¹H NMR (300 MHz, d₆-DMSO): δ: 10.58 (s, 1H), 8.77 (s, 1H), 8.32 (d, 1H), 7.32 (d, 1H), 7.19 (d, 1H), 7.03 (d, 1H), 5.90-5.78 (m, 1H), 5.72-5.59 (m, 1H), 4.04 (s, 3H), 3.65 (s, 2H), 3.45 (s, 2H), 3.42-3.35 (m, 1H), 3.47-2.93 (m, 4H), 1.94-1.58 (m, 2H), 1.56-1.42 (m, 2H), 1.39-1.10 (m, 3H)

### Example 29

### 6-({8-[2-(3-Chloro-6-methoxy-[1,5]-naphthyridin-4-yl)-2-hydroxy-ethyl]-8-aza-bicyclo[3.2.1]oct-3-ylamino}-methyl)-4H-benzo[1,4]thiazin-3-one

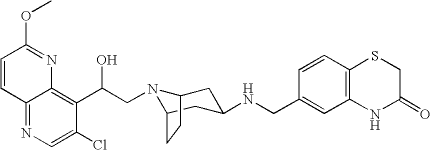

The compound was prepared as in example 1k from aldehyde (6b).

¹H NMR (300 MHz, d₆-DMSO): δ: 10.54 (s, 1H), 8.76 (s, 1H), 8.32 (d, 1H), 7.32 (d, 1H), 7.23 (d, 1H), 6.97-6.87 (m, 2H), 5.85-5.75 (m, 1H), 5.69-5.52 (m, 1H), 4.04 (s, 3H), 3.68 (s, 2H), 3.42 (s, 2H), 3.40-3.30 (m, 1H), 3.20-2.90 (m, 3H), 2.85-2.64 (m, 1H), 1.92-1.54 (m, 4H), 1.50-1.05 (m, 5H)

### Example 30

### 1-(3-Chloro-6-methoxy-[1,5]-naphthyridin-4-yl)-2-{3-[(2,3-dihydro-[1,4]dioxino[2,3-c]pyridin-7-ylmethyl)-amino]-8-aza-bicyclo[3.2.1]oct-8-yl}-ethanol

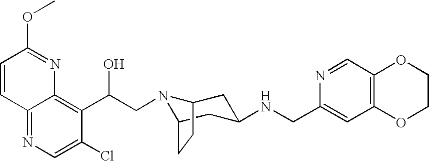

### 30a) 5-Benzyloxy-2-hydroxymethyl-pyran-4-one

To a solution of kojic acid (10.36 g) in warm methanol (135 ml) was added sodium methoxide (4.3 g) in portions and benzyl chloride (9.6 ml) in one portion. The mixture was heated to 70° C. overnight and cooled down to room temperature. The reaction mixture was poured onto ice-water. The solid was filtered off and dried to give the desired product (6.43 g).

¹H NMR (300 MHz, d₆-DMSO): δ: 8.18 (s, 1H), 7.44-7.32 (m, 5H), 6.33 (s, 1H), 5.71-5.66 (m, 1H), 4.95 (s, 2H), 4.30 (d, 2H)

### 30b) 5-Benzyloxy-2-hydroxymethyl-1H-pyridin-4-one

A mixture of the pyranone (30a) (6.43 g) and concentrated aqueous ammonia (67 ml) in ethanol (14 ml) was heated to reflux overnight. The solution was cooled to room temperature, the solid filtered off and dried to give the desired product (5.1 g).

¹H NMR (300 MHz, d₆-DMSO): δ: 11.17 (bs, 1H), 7.48-7.29 (m, 5H), 6.14 (bs, 1H), 5.59 (bs, 1H), 5.02 (s, 2H), 4.34 (s, 2H)

### 30c) (2,3-Dihydro-[1,4]dioxino[2,3-c]pyridin-7-yl)-methanol

A solution of pyridinone (30b) (12.6 g) in water (1.4 l) containing sodium hydroxide (4.36 g) was hydrogenated over 10% palladium on charcoal (6.7 g) for 2 days. The mixture was filtered and the filtrate lyophilised. The residue was dissolved in DMF (106 ml) and treated with potassium carbonate (18.13 g) and 1,2-dibromoethane (3.84 ml). The reaction mixture was heated at 100° C. overnight, cooled to room temperature and concentrated. The residue was partitioned between water and ethyl acetate. The aqueous layer was back extracted twice with ethyl acetate, dried over magnesium sulfate, filtered and concentrated. The residue was purified by flash chromatography (silica gel, dichloromethane/methanol 9:1) to give the desired product (1.49 g).

¹H NMR (300 MHz, d₆-DMSO): δ: 8.00 (s, 1H), 6.91 (s, 1H), 5.31-5.26 (m, 1H), 4.41 (d, 2H), 4.36-4.33 (m, 2H), 4.29-4.26 (m, 2H)

### 30d) 2,3-Dihydro-[1,4]dioxino[2,3-c]pyridine-7-carbaldehyde

To a solution of oxalyl chloride (2.2 ml) in dichloromethane (22 ml) cooled to −78° C. was added dropwise a solution of DMSO (2.2 ml) in dichloromethane (22 ml). The reaction mixture was stirred for 15 minutes, and then a solution of alcohol (30c) (1.49 g) in dichloromethane (16 ml) was added. After stirring for 1 hour at this temperature, a solution of triethylamine (8.7 ml) in dichloromethane (11 ml) was added. The reaction was stirred for 20 minutes, then warmed to 0° C. and stirred for 30 minutes. Water was added and the layers were separated. The aqueous layer was extracted twice with dichloromethane. The combined organic layers were dried over magnesium sulfate, filtered and evaporated. The residue was purified by flash chromatography (silica gel, dichloromethane/methanol 19:1) to give the desired product (1.36 g).

¹H NMR (300 MHz, CDCl₃): δ: 9.91 (s, 1H), 8.24 (s, 1H), 7.45 (s, 1H), 4.33 (s, 4H)

### 30e) Title Compound

The compound was prepared as in example 1k from aldehyde (30d).

¹H NMR (300 MHz, d₆-DMSO): δ: 8.76 (s, 1H), 8.32 (d, 1H), 8.03 (s, 1H), 7.32 (d, 1H), 6.93 (s, 1H), 5.86-5.76 (m, 1H), 5.69-5.55 (m, 1H), 4.35-4.33 (m, 2H), 4.30-4.27 (m, 2H), 4.04 (s, 3H), 3.73 (s, 2H), 3.41-3.30 (m, 1H), 2.31-2.79 (m, 4H), 1.90-1.55 (m, 4H), 1.46-1.22 (m, 5H)

### Example 31

### 2-(2-{[(2,3-Dihydro-benzo[1,4]dioxin-6-ylmethyl)-amino]-methyl}-morpholin-4-yl)-1-(6-methoxy-[1,5]naphthyridin-4-yl)-ethanol

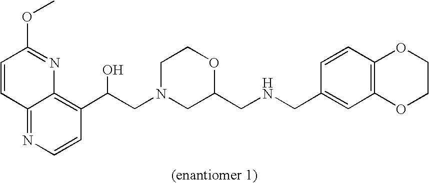

### 31a) (4-Benzyl-morpholin-2-ylmethyl)-carbamic acid tert-butyl ester

(4-Benzyl-1,4-oxazinan-2-yl)methylamine (4 g) were dissolved in absolute dichloromethane (100 ml) followed by the addition of triethylamine (5.4 ml) and di-tert-butyl dicarbonate (5.085 g) at room temperature. The mixture was stirred for 1 hour at room temperature, and then the solvents were evaporated. The residue was purified by flash chromatography (silica gel, ethyl acetate/n-heptane 4:1) to give the desired product (5.9 g).

MS (EI): m/z: 317 [M+H]⁺

### 31b) Morpholin-2-ylmethyl-carbamic acid tert-butyl ester

(4-Benzyl-morpholin-2-ylmethyl)-carbamic acid tert-butyl ester (31a) (5.9 g) were dissolved in methanol/THF (1:1, 100 ml). To the solution was added 10% palladium on charcoal (2.8 g) and the flask set under a hydrogen atmosphere and stirred for 2 hours. After completion of the reaction the catalyst was removed by filtration via silica gel and the resulting solution evaporated to dryness to give the desired product (3.5 g).

MS (EI): m/z: 217 [M+H]⁺

### 31c) {4-[2-Hydroxy-2-(6-methoxy-[1,5]naphthyridin-4-yl)-ethyl]-morpholin-2-ylmethyl}-carbamic acid tert-butyl ester (enantiomer 1)

Naphthyridin-epoxide (1f) (200 mg) and morpholin-2-ylmethyl-carbamic acid tert-butyl ester (31b) (214 mg) were dissolved in DMF (3 ml), treated with potassium carbonate (144 mg) and lithium perchlorate (105 mg) and stirred at 80° C. for 4 days. The mixture was concentrated, dissolved in dichloromethane/methanol 9:1 and extracted with water and brine. The organic layer was dried over magnesium sulfate, filtered and concentrated. The residue was purified by flash chromatography (silica gel, dichloromethane/(methanol/ammonia 9:1) 9:1) to give the desired product (329 mg).

¹H NMR (300 MHz, CDCl₃): δ: 8.84-8.76 (m, 1H), 8.25 (dd, 1H), 7.86-7.78 (m, 1H), 7.14 (m, 1H), 6.96 (bd, 1H), 5.96-5.84 (m, 1H), 4.11-3.93 (m, 2H), 4.05 (s, 3H), 3.54-3.03 (m, 5H), 2.86-2.56 (m, 2H), 2.54-2.37 (m, 1H), 1.46 (s, 9H)

### 31d) 2-(2-Aminomethyl-morpholin-4-yl)-1-(6-methoxy-[1,5]naphthyridin-4-yl)-ethanol (enantiomer 1)

Boc-amine (31a) (329 mg) was dissolved in dichloromethane (6 ml), treated with TFA (0.6 ml) and stirred at room temperature over night. The mixture was made alkaline with 2N sodium hydroxide solution and the layers were separated. The aqueous layer was extracted once more with dichloromethane. The combined organic layers were washed with brine, dried over magnesium sulfate, filtered and concentrated. The residue was purified by flash chromatography (silica gel, dichloromethane/(methanol/ammonia 9:1) 8:2) to give the desired product (172 mg).

¹H NMR (300 MHz, CDCl₃): δ: 8.82-8.75 (m, 1H), 8.22 (dd, 1H), 7.82-7.75 (m, 1H), 7.11 (dd, 1H), 5.76 (bd, 1H), 4.04 (s, 3H), 4.02-3.68 (m, 6H), 3.39-2.86 (m, 4H), 2.84-2.62 (m, 1H), 2.60-2.06 (m, 3H)

### 31e) Title Compound

The compound was prepared as in example 1k from 2,3-dihydro-benzo[1,4]dioxine-6-carbaldehyde.

¹H NMR (300 MHz, d₆-DMSO): δ: 8.78 (d, 1H), 8.26 (d, 1H), 7.76 (d, 1H), 7.26 (d, 1H), 6.83-6.69 (m, 3H), 5.88-5.76 (m, 1H), 5.38-5.29 (m, 1H), 4.23 (s, 3H), 3.98 (s, 2H), 3.82-3.69 (m, 1H), 3.60-3.42 (m, 5H), 3.20-3.10 (m, 1H), 3.03-2.94 (m, 1H), 2.89-2.81 (m, 1H), 2.77-2.63 (m, 2H), 2.54-2.35 (m, 3H), 2.30-2.15 (m, 1H), 2.05-1.91 (m, 1H)

### Example 32

### 6-[({4-[2-Hydroxy-2-(6-methoxy-[1,5]naphthyridin-4-yl)-ethyl]-morpholin-2-ylmethyl}amino)-methyl]-4H-benzo[1,4]thiazin-3-one

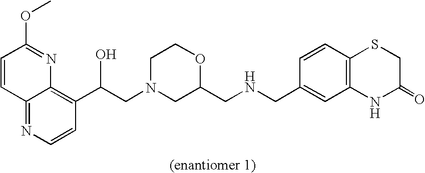

The compound was prepared as in example 1k from aldehyde (6b).

MS (EI): m/z: 496 [M+H]⁺

### Example 33

### 6-[({4-[2-Hydroxy-2-(6-methoxy-[1,5]naphthyridin-4-yl)-ethyl]-morpholin-2-ylmethyl}amino)-methyl]-4H-pyrido[3,2-b][1,4]thiazin-3-one

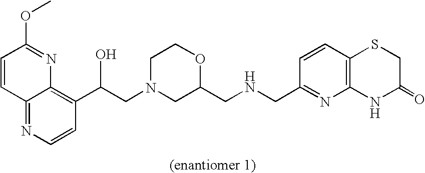

The compound was prepared as in example 1k from aldehyde (17h).

MS (EI): m/z: 497 [M+H]⁺

### Example 34

### 6-[({4-[2-Hydroxy-2-(6-methoxy-[1,5]naphthyridin-4-yl)-ethyl]-morpholin-2-ylmethyl}amino)-methyl]-4H-benzo[1,4]oxazin-3-one

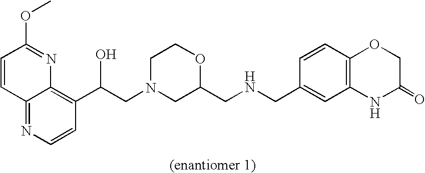

The compound was prepared as in example 1k from aldehyde (1j).

MS (EI): m/z: 480 [M+H]⁺

### Example 35

### 1-(3-Methoxy-quinolin-5-yl)-2-[3-((E)-3-pyridin-2-yl-allylamino)-8-aza-bicyclo[3.2.1]oct-8-yl]-ethanol (enantiomer 1)

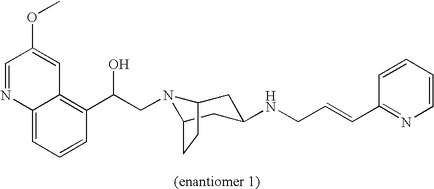

### 35a) 3,5-Dibromo-quinoline

3-Bromoquinoline (250 g) was added dropwise to stirred ice cold concentrated sulphuric acid (625 ml) ensuring that the temperature did not rise above 15° C. N-Bromosuccinimide (240 g) was added slowly in portions, such that the temperature did not rise above 20° C., and the mixture was allowed to stir overnight. The solution was poured carefully onto ice (10 kg) and made alkaline with sodium hydroxide pellets, with cooling. The resulting mixture was filtered, the solid washed with water and dried in a vacuum oven at 40° C. Methanol (1.5 l) was added to the crude dried solid. The resulting mixture was refluxed, cooled, filtered and the solid washed with cold methanol (500 ml). The filtrate was evaporated and the product purified by flash chromatography (silica gel, ethyl acetate/n-heptane 1:29 to 1:19 to 1:9) to give the desired product (151 g).

¹H NMR (300 MHz, CDCl₃): δ: 8.85 (d, 1H), 8.65-8.64 (m, 1H), 7.99 (d, 1H), 7.78 (d, 1H), 7.56-7.49 (m, 1H)

### 35b) 5-Bromo-3-methoxy-quinoline

3,5-dibromoquinoline (35a) (150 g) was added to a stirred mixture of sodium methoxide (35.78 g) in dry DMPU (1.5 l) at 100° C. The resulting mixture was heated at 125° C. for 90 minutes, cooled to room temperature, poured onto ice (7.5 kg) and stirred overnight. The suspension was filtered, the solid washed with water and dried in a vacuum oven at 40° C. The product was purified by flash chromatography (silica gel, n-heptane/ethyl acetate 19:1 to 4:1) to give the desired product (65.2 g).

¹H NMR (300 MHz, CDCl₃): δ: 8.60 (d, 1H), 7.95 (d, 1H), 7.72 (d, 1H), 7.65 (d, 1H), 7.37-7.31 (m, 1H), 3.93 (s, 3H)

### 35c) 3-Methoxy-5-vinyl-quinoline

Tetrakis(triphenylphosphine) palladium (1.155 g) was added to a stirred solution of 5-bromo-3-methoxy quinoline (35b) (9.52 g) in dry dimethoxy ethane (450 ml) under nitrogen at room temperature and the resulting mixture stirred for 20 minutes. Anhydrous potassium carbonate (5.57 g), water (120 ml) and 2,4,6-trivinylcycloboroxane pyridine complex (3.85 g,—O'Sheas reagent—See J. Org. Chem., Vol. 67 (2002), 4968-71) were then added and the mixture heated to 100° C. for 4 hours. After cooling to room temperature, water (200 ml) was added and the mixture extracted with ethyl acetate (4×150 ml). The combined organic extracts were dried over sodium sulfate, filtered and evaporated. The product was purified by flash chromatography (silica gel, n-heptane/ethyl acetate 9:1 to 3:2) to give the desired product (7.41 g).

¹H NMR (300 MHz, CDCl₃): δ: 8.60 (d, 1H), 7.91 (d, 1H), 7.57-7.41 (m, 3H), 7.28-7.22 (m, 1H), 5.72 (dd, 1H), 5.43 (dd, 1H), 3.87 (s, 3H)

### 35d) 1-(3-Methoxy-quinolin-5-yl)-ethane-1,2-diol (enantiomer 1)

AD mix beta (90.2 g) and methanesulfonamide (7.6 g) were added to water (280 ml) and tert-butanol (280 ml) at room temperature. To the cooled (0° C.) orange solution was added vinyl quinoline (35c) (14.4 g) and the mixture stirred at 0-4° C. for 2 days. To the mixture was added sodium metabisulfite (108 g) at 0° C., stirred for 30 minutes at this temperature and then warmed to room temperature. The mixture was extracted with ethyl acetate (5×150 ml) and the combined organic extracts were dried over sodium sulfate, filtered and evaporated. The crude product was purified by flash chromatography (silica gel, dichloromethane/methanol 29:1 to 4:1) to give the desired product (14.91 g).

¹H NMR (300 MHz, d₆-DMSO): δ: 8.65 (d, 1H), 7.88-7.85 (m, 2H), 7.66 (d, 1H), 7.58-7.53 (m, 1H), 5.51 (d, 1H), 5.31-5.26 (m, 1H), 4.87-4.84 (m, 1H), 3.96 (s, 3H), 3.67-3.57 (m, 2H)

### 35e) Toluene-4-sulfonic acid 2-hydroxy-2-(3-methoxy-quinolin-5-yl)-ethyl ester (enantiomer 1)

Dibutyl tin oxide (0.33 g), para toluene sulfonic acid (12.78 g) and triethylamine (9.33 ml) were added to a stirred suspension of diol (35d) (14.4 g) in dry dichloromethane (150 ml) at room temperature. The reaction was stirred for 4 hours, quenched with water (150 ml) and the layers were separated. The aqueous layer was back extracted with dichloromethane (2×150 ml) and the combined organic extracts were washed with water (150 ml) and brine (150 ml), dried over sodium sulfate, filtered and evaporated. The crude product was purified by flash chromatography (silica gel, dichloromethane/methanol 9:1) to give the desired product (16.12 g).

¹H NMR (300 MHz, d₆-DMSO): δ: 8.63 (d, 1H), 7.89 (d, 1H), 7.67-7.62 (m, 2H), 7.58-7.47 (m, 3H), 7.27 (d, 2H), 6.05 (bs, 1H), 5.56 (bs, 1H), 4.25 (dd, 1H), 4.14 (dd, 1H), 3.89 (s, 3H), 2.34 (s, 3H)

### 35f) 3-Methoxy-5-oxiranyl-quinoline (enantiomer 1)

Tosylate (35e) (5.15 g) was dissolved in DMF (69 ml), cooled with an ice bath and stirred for 10 minutes. Then sodium hydride (661 mg) was added and the mixture stirred for 15 minutes at 0° C., then 90 minutes at room temperature. The mixture was diluted with ether and extracted with water and brine. The organic layer was dried over magnesium sulfate, filtered and concentrated. The crude product was purified by flash chromatography (silica gel, ethyl acetate/hexane 1:9) to give the desired product (2.12 g).

¹H NMR (300 MHz, CDCl₃): δ: 8.64 (d, 1H), 7.94 (dd, 1H), 7.59 (d, 1H), 7.48-7.39 (m, 2H), 4.30 (m, 1H), 3.91 (s, 3H), 3.22 (dd, 1H), 2.81 (dd, 1H)

### 35g) {8-[2-Hydroxy-2-(3-methoxy-quinolin-5-yl)-ethyl]-8-aza-bicyclo[3.2.1]oct-3-yl}-carbamic acid tert-butyl ester (enantiomer 1)

Epoxide (35f) (500 mg) was dissolved in DMF (13 ml), treated with amine (8g) (562 mg) and lithium perchlorate (317 mg) and stirred at 80° C. over night. The mixture was diluted with ethyl acetate and washed with water and brine. The organic layer was dried over magnesium sulfate, filtered and evaporated. The residue was purified by flash chromatography (silica gel, dichloromethane, dichloromethane/methanol 19:1) to give the desired product (808 mg).

¹H NMR (300 MHz, CDCl₃): δ: 8.55 (d, 1H), 7.90 (d, 1H), 7.65-7.62 (m, 2H), 7.48-7.43 (m, 1H), 5.66 (bs, 1H), 4.58 (bs, 1H), 3.85 (s, 3H), 3.53-3.50 (m, 1H), 2.85-2.80 (m, 1H), 2.70-2.54 (m, 1H), 2.03-1.73 (m, 9H), 1.36 (s, 9H), 1.30-1.15 (m, 2H)

### 35h) 2-(3-Amino-8-aza-bicyclo[3.2.1]oct-8-yl)-1-(3-methoxyquinolin-5-yl)-ethanol (enantiomer 1)

Boc-amine (35g) (808 mg) was dissolved in dichloromethane (7 ml), treated with TFA (1.4 ml) and stirred at room temperature over night. The mixture was made alkaline with 2N sodium hydroxide solution. The aqueous layer was extracted once with dichloromethane. The organic layer washed with water and brine, dried over magnesium sulfate, filtered and concentrated. The residue was purified by flash chromatography (silica gel, dichloromethane/methanol 9:1+1% ammonia) to give the desired product (366 mg).

¹H NMR (300 MHz, d₆-DMSO): δ: 8.65 (d, 1H), 7.87-7.84 (m, 2H), 7.69 (d, 1H), 7.58-7.53 (m, 1H), 5.35-5.30 (m, 1H), 5.21 (bs, 1H), 3.96 (s, 3H), 3.25-3.12 (m, 2H), 2.82-2.68 (m, 2H), 2.60-2.56 (m, 1H), 1.88-1.70 (m, 2H), 1.59-1.15 (m, 8H)

### 35i) Title Compound

The compound was prepared as in example 1k from aldehyde (5a).

¹H NMR (300 MHz, d₆-DMSO): δ: 8.58 (d, 1H), 8.46-8.44 (m, 1H), 7.89 (d, 1H), 7.63-7.61 (m, 2H), 7.57-7.43 (m, 2H), 7.06-7.02 (m, 1H), 6.72-6.52 (m, 2H), 3.88 (s, 3H), 3.45-3.41 (m, 1H), 3.39 (s, 4H), 3.35-3.24 (m, 1H), 2.91-2.81 (m, 2H), 2.54-2.46 (m, 1H), 1.91-1.77 (m, 4H), 1.67-1.52 (m, 4H)

### Example 36

### 6-({8-[2-Hydroxy-2-(3-methoxy-quinolin-5-yl)-ethyl]-8-aza-bicyclo[3.2.1]oct-3-ylamino}-methyl)-4H-benzo[1,4]thiazin-3-one (enantiomer 1)

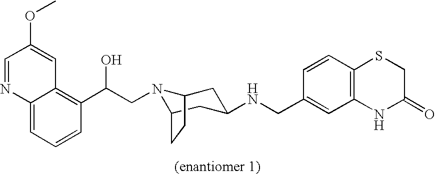

The compound was prepared as in example 1k from aldehyde (6b).

¹H NMR (300 MHz, d₆-DMSO): δ: 10.64 (s, 1H), 8.66 (d, 1H), 7.91-7.84 (m, 2H), 7.73 (d, 1H), 7.61-7.55 (m, 1H), 7.30 (d, 1H), 7.03-6.98 (m, 2H), 5.76 (s, 1H), 5.51 (bs, 1H), 3.98 (s, 3H), 3.95-3.84 (m, 1H), 3.79 (s, 2H), 3.66-3.48 (m, 1H), 3.45 (s, 2H), 3.16-2.96 (m, 1H), 2.94-2.67 (m, 2H), 2.03-1.44 (m, 9H)

### Example 37

### 6-({8-[2-Hydroxy-2-(3-methoxy-quinolin-5-yl)-ethyl]-8-aza-bicyclo[3.2.1]oct-3-ylamino}-methyl)-4H-pyrido[3,2-b][1,4]thiazin-3-one (enantiomer 1)

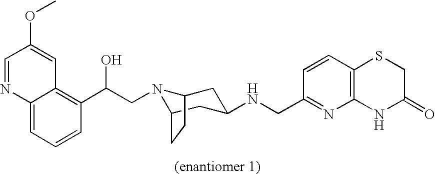

The compound was prepared as in example 1k from aldehyde (17h).

¹H NMR (300 MHz, d₆-DMSO): δ: 10.89 (s, 1H), 8.66 (d, 1H), 7.91-7.84 (m, 2H), 7.78-7.71 (m, 1H), 7.60-7.55 (m, 1H), 7.08 (d, 1H), 5.76 (s, 1H), 5.50 (bs, 1H), 3.97 (s, 3H), 3.75 (s, 2H), 3.54 (s, 2H), 3.50-3.26 (m, 4H), 2.98-2.66 (m, 2H), 1.95-1.40 (m, 8H)

### Example 38

### 1-(3-Methoxy-quinolin-5-yl)-2-[3-((E)-3-phenylallylamino)-8-aza-bicyclo[3.2.1]oct-8-yl]-ethanol

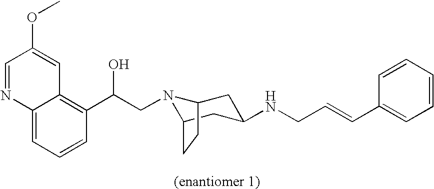

The compound was prepared as in example 1k from cinnamic aldehyde.

MS (EI): m/z: 444 [M+H]⁺

### Example 39

### 2-{3-[(2,3-Dihydro-benzo[1,4]dioxin-6-ylmethyl)-amino]-8-aza-bicyclo[3.2.1]oct-8-yl}-1-(3-methoxy-quinolin-5-yl)-ethanol

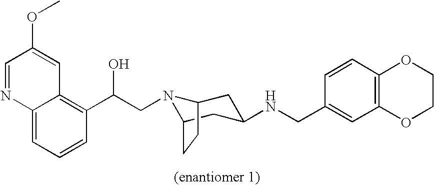

The compound was prepared as in example 1k from 2,3-dihydro-benzo[1,4]dioxine-6-carbaldehyde.

MS (EI): m/z: 476 [M+H]⁺

### Example 40

### 2-{3-[(Benzo[1,2,5]thiadiazol-5-ylmethyl)-amino]-8-aza-bicyclo[3.2.1]oct-8-yl}-1-(3-methoxy-quinolin-5-yl)-ethanol

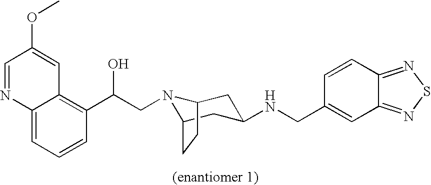

The compound was prepared as in example 1k from benzo[1,2,5]thiadiazole-5-carbaldehyde.

MS (EI): m/z: 476 [M+H]⁺

### Example 41

### 2-(3-{[(Benzo[1,2,5]thiadiazol-5-ylmethyl)-amino]-methyl}-piperidin-1-yl)-1-(3-methoxy-quinolin-5-yl)-ethanol (enantiomer 1)

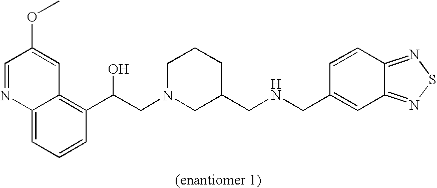

### 41a) {1-[2-Hydroxy-2-(3-methoxy-quinolin-5-yl)-ethyl]-piperidin-3-ylmethyl}-carbamic acid tert-butyl ester (enantiomer 1)

To a solution of epoxide (35f) (500 mg) and (3-Bocaminomethyl)piperidine (533 mg) in DMF (10 ml) was added lithium perchlorate (317 mg), and heated at reflux over night. The mixture was dissolved in water (150 ml) and extracted three times with ethyl acetate. The combined organic layers were washed with brine, dried over magnesium sulfate, filtered and evaporated. The crude product was purified by flash chromatography (silica gel dichloromethane/methanol 9:1) to give the desired product (934 mg).

MS (EI): m/z: 416 [M+H]⁺

### 41b) 2-(3-Aminomethyl-piperidin-1-yl)-1-(3-methoxy-quinolin-5-yl)-ethanol (enantiomer 1)

To a solution of Boc-amine (41a) (900 mg) in dichloromethane (15 ml) was added trifluoroacetic acid (8 ml). The mixture was stirred for 20 minutes at room temperature and then concentrated. Dichloromethane (10 ml) and 2N sodium hydroxide solution (30 ml) were added. The aqueous layer was back extracted three times with dichloromethane. The combined organic layers were dried over magnesium sulfate, filtered and evaporated to give the desired product (634 mg).

MS (EI): m/z: 316 [M+H]⁺

### 41c) Title Compound

The compound was prepared as in example 1k from benzo[1,2,5]thiadiazole-5-carbaldehyde.

MS (EI): m/z: 464 [M+H]⁺

### Example 42

### 6-[({1-[2-Hydroxy-2-(3-methoxy-quinolin-5-yl)-ethyl]-piperidin-3-ylmethyl}-amino)-methyl]-4H-pyrido[3,2-b][1,4]thiazin-3-one (enantiomer 1)

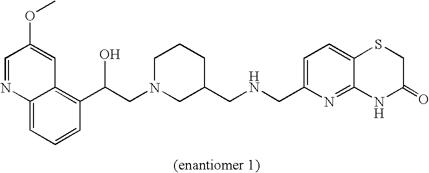

The compound was prepared as in example 1k from aldehyde (17h).

MS (EI): m/z: 494 [M+H]⁺

### Example 43

### 2-(3-{[(2,3-Dihydro-[1,4]dioxino[2,3-c]pyridin-7-ylmethyl)-amino]-methyl}-piperidin-1-yl)-1-(3-methoxy-quinolin-5-yl)-ethanol (enantiomer 1)

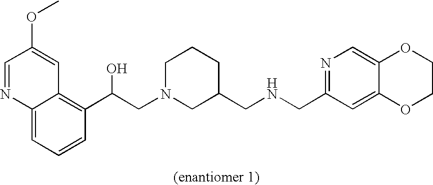

The compound was prepared as in example 1k from aldehyde (30d).

MS (EI): m/z: 465 [M+H]⁺

### Example 44

### 2-(3-{[(Benzo[1,3]dioxol-5-ylmethyl)-amino]-methyl}-piperidin-1-yl)-1-(3-methoxy-quinolin-5-yl)-ethanol (enantiomer 1)

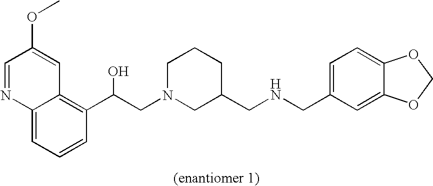

The compound was prepared as in example 1k and benzo[1,3]dioxole-5-carbaldehyde.

MS (EI): m/z: 450 [M+H]⁺

### Example 45

### 6-[({1-[2-Hydroxy-2-(3-methoxy-quinolin-5-yl)-ethyl]-piperidin-3-ylmethyl}-amino)-methyl]-4H-benzo[1,4]oxazin-3-one (enantiomer 1)

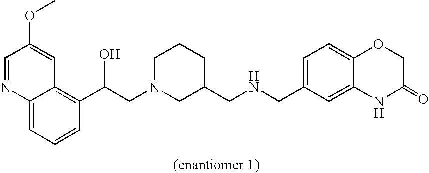

The compound was prepared as in example 1k from aldehyde (1j).

MS (EI): m/z: 477 [M+H]⁺

### Example 46

### 3-{[(7-Fluoro-3-oxo-3,4-dihydro-2H-benzo[1,4]thiazin-6-ylmethyl)-amino]-methyl}-cyclohexanecarboxylic acid (3-methoxy-quinolin-5-yl)-amide

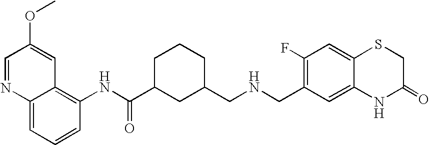

### 46a) (3-Carbamoyl-cyclohexylmethyl)-carbamic acid tert-butyl ester

HOBT ammonium salt (4.02 g) was added to a stirred solution of 3-(tert-butoxycarbonylamino-methyl)cyclohexane carboxylic acid (5.14 g—Prepared according to the method of Yang, J. Med. Chem., 1998, 2175-2179) in dry DMF at room temperature. The solution was stirred for 12 hours and the solvent was evaporated. The crude mixture was taken up in ethyl acetate (500 ml), washed with water (250 ml), saturated sodium bicarbonate solution (250 ml) and brine (250 ml), dried over sodium sulfate, filtered and evaporated to give the desired product (4.48 g) which was used directly for the next step.

### 46b) [3-(3-Methoxy-quinolin-5-ylcarbamoyl)-cyclohexylmethyl]-carbamic acid tert-butyl ester

A mixture of amide (46a) (1.5 g), caesium carbonate (2.44 g), tris(dibenzylideneacetone) dipalladium (0) chloroform complex (0.108 g) and 4,5-bis(diphenylphosphino)-9,9-dimethylxanthene (0.208 g) in dry dioxane (50 ml) under nitrogen atmosphere was sonicated for 10 minutes, during which the solution turned brown. To this solution was added 5-bromo-3-methoxy quinoline (35b) (1.8 g) and the mixture was heated at 100° C. for 24 hours. After cooling to room temperature, the mixture was centrifuged and the supernatant removed and evaporated. The product was purified by flash chromatography (silica gel, ethyl acetate/n-heptane 3:2) to give the desired product (1.84 g).

¹H NMR (300 MHz, d₆-DMSO): δ: 9.84 (s, 1H), 8.67 (d, 1H), 7.79 (d, 1H), 7.71-7.68 (m, 2H), 7.57-7.52 (m, 1H), 6.90-6.86 (m, 1H), 3.95 (s, 3H), 2.94-2.72 (m, 2H), 2.64-2.50 (m, 1H), 2.02-1.79 (m, 3H), 1.77-1.64 (m, 1H), 1.59-1.41 (m, 1H), 1.38 (s, 9H), 1.24-1.05 (m, 2H), 0.96-0.79 (m, 1H)

### 46c) 3-Aminomethyl-cyclohexanecarboxylic acid (3-methoxyquinolin-5-yl)-amide

Sieves 3A (876 mg) were suspended in dry dichloromethane (15 ml), cooled with an ice/water bath and treated with a solution of Boc-amine (46b) (600 mg) in dry dichloromethane (8 ml). Then boron trifluoride etherate (0.152 ml) in dry dichloromethane (1.3 ml) was added over a period of 45 minutes. The mixture was stirred at room temperature over night. The sieves were filtered off and washed with ethyl acetate, dichloromethane and methanol. The mixture was concentrated and treated with dichloromethane/methanol 9:1. The precipitate was filtered off and washed with pentane to give the desired product (454 mg).

¹H NMR (300 MHz, d₆-DMSO): δ: 9.91 (s, 1H), 8.76 (d, 1H), 7.86-7.55 (m, 6H), 3.98 (s, 3H), 2.83-2.56 (m, 3H), 2.10-1.60 (m, 5H), 1.52-1.19 (m, 3H), 1.08-0.90 (m, 1H)

### 46d) Title Compound

The compound was prepared as in example 1k from aldehyde (24g).

MS (EI): m/z: 509 [M+H]⁺

### Example 47

### 3-{[(3-Oxo-3,4-dihydro-2H-benzo[1,4]thiazin-6-ylmethyl)-amino]-methyl}-cyclohexanecarboxylic acid (3-methoxyquinolin-5-yl)-amide

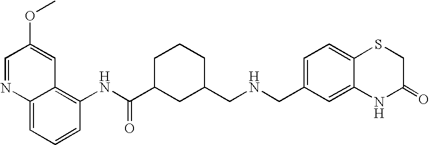

The compound was prepared as in example 1k from aldehyde (6b).

MS (EI): m/z: 491 [M+H]⁺

### Example 48

### 3-{[(3-Oxo-3,4-dihydro-2H-benzo[1,4]oxazin-6-ylmethyl)-amino]-methyl}-cyclohexanecarboxylic acid (6-methoxyquinolin-4-yl)-amide

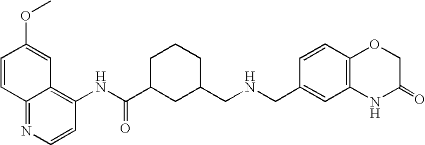

### 48a) 6-Methoxy-quinolin-4-ol

To a solution of p-anisidine (20 g) in ethanol (120 ml) was added triethylorthoformate (27.2 ml) and meldrums acid (27.4 g). The mixture was heated to reflux for 2 hours. Then the mixture was cooled, filtered and washed with ethanol. The intermediate was dried under vacuum over night.

The intermediate (38.9 g) was added in portions to boiling diphenyl ether (250 g). 2 minutes after completion of addition, the mixture was cooled, diluted with diethyl ether and ethyl acetate and filtered. The precipitate washed with ethyl acetate and dried under vacuum to give the desired product (21.7 g).

¹H NMR (300 MHz, d₆-DMSO): δ: 11.75 (bs, 1H), 7.87-7.83 (m, 1H), 7.52-7.49 (m, 2H), 7.31-7.27 (m, 1H), 6.00 (d, 1H), 3.83 (s, 3H)

### 48b) 4-Chloro-6-methoxy-quinoline

A solution of phenol (48a) (1.35 g) in phosphorous oxychloride (3 ml) was heated at 80° C. for 2 hours. After cooling, water was added and the resulting solution was made alkaline by adding 6N sodium hydroxide solution. The precipitated solid was filtered off and washed with water. The precipitate was taken up in diethyl ether and filtered. The diethyl ether layer was dried over magnesium sulfate, filtered and evaporated to give the desired product (1 g).

¹H NMR (300 MHz, CDCl₃): δ: 8.66 (d, 1H), 8.06 (d, 1H), 7.51-7.43 (m, 3H), 4.01 (s, 3H)

### 48c) 6-Methoxy-quinolin-4-ylamine

To a solution of chloride (48b) (3.0 g) in pyridine (50 ml) was added n-propylamine hydrochloride (9.6 g). The mixture was then refluxed for 40 hours. The solvent was removed in vacuo and the residue was partitioned between water (30 ml) and ethyl acetate (50 ml). The solution was made alkaline by adding 1M sodium hydroxide solution. The aqueous layer was then back extracted with ethyl acetate (4×50 ml) and the combined organic layers were washed with brine, dried over magnesium sulfate, filtered and evaporated. The residue was purified by flash chromatography (silica gel, dichloromethane/methanol 9:1+1% ammonia) to afford the desired product (2.4 g).

¹H NMR (300 MHz, d₆-DMSO): δ: 8.19 (d, 1H), 7.67 (d, 1H), 7.49 (d, 1H), 7.24 (dd, 1H), 6.60 (bs, 2H), 6.51 (d, 1H), 3.87 (s, 3H)

### 48d) [3-(6-Methoxy-quinolin-4-ylcarbamoyl)-cyclohexylmethyl]-carbamic acid tert-butyl ester

Quinoline amine (48c) (1.74 g) and 3-(tert-Butoxycarbonylaminomethyl)-cyclohexanecarboxylic acid (2.57 g—Prepared according to the method of Yang, J. Med. Chem., 1998, 2175-2179) were dissolved in DMF (50 ml), then HBTU (3.8 g) and triethylamine (2.8 ml) were added and the mixture heated at 60° C. over night. The solvent was evaporated and the residue partitioned between ethyl acetate and brine. The organic layer was dried over magnesium sulfate, filtered and evaporated. The crude product was purified by flash chromatography (silica gel, dichloromethane/(methanol/ammonia 9:1) 19:1) to give the desired product (3.42 g).

¹H NMR (300 MHz, CDCl₃): δ: 8.78 (bs, 1H), 8.46 (d, 1H), 8.14 (d, 1H), 7.93 (d, 1H), 7.26-7.23 (m, 2H), 4.71-4.67 (m, 1H), 3.93 (s, 3H), 2.98-2.93 (m, 2H), 2.73-2.63 (m, 1H), 2.07-1.93 (m, 2H), 1.92-1.80 (m, 1H), 1.78-1.68 (m, 1H), 1.61-1.42 (m, 2H), 1.36 (s, 9H), 1.26-1.11 (m, 2H), 0.97-0.81 (m, 1H)

### 48e) 3-Aminomethyl-cyclohexanecarboxylic acid (6-methoxyquinolin-4-yl)-amide

Compound (48d) (3.42 g) was dissolved in dichloromethane (198 ml), 3A sieves (5.2 g) were added and then boron trifluoride etherate (5.2 ml) under ice bath cooling over a period of 25 minutes. The mixture was stirred at room temperature over night. The sieves were filtered off and washed with ethyl acetate, dichloromethane and methanol. The filtrate was evaporated and the residue purified by flash chromatography (silica gel, dichloromethane/methanol 9:1+1% ammonia) to give the desired product (2.43 g).

¹H NMR (300 MHz, d₆-DMSO): δ: 10.01 (bs, 1H), 8.63 (d, 1H), 8.00 (d, 1H), 7.91 (d, 1H), 7.61 (d, 1H), 7.43 (dd, 1H), 5.48 (bs, 2H), 3.96 (s, 3H), 2.81-2.73 (m, 1H), 2.61 (d, 2H), 2.07-1.72 (m, 4H), 1.65-1.47 (m, 1H), 1.45-1.31 (m, 2H), 1.28-1.10 (m, 1H), 1.01-0.85 (m, 1H)

### 48f) Title Compound

The compound was prepared as in example 1k from aldehyde (1j).

¹H NMR (300 MHz, d₆-DMSO): δ: 10.79 (s, 1H), 10.06 (s, 1H), 8.68 (d, 1H), 8.05 (d, 1H), 7.97 (d, 1H), 7.67 (d, 1H), 7.48 (dd, 1H), 7.00-6.93 (m, 3H), 4.59 (s, 2H), 4.00 (s, 3H), 3.76 (s, 2H), 3.46 (bs, 1H), 2.85-2.78 (m, 1H), 2.10-1.96 (m, 2H), 1.90-1.81 (m, 2H), 1.76-1.59 (m, 1H), 1.56-1.35 (m, 2H), 1.32-1.14 (m, 2H), 1.06-0.88 (m, 1H)

### Example 49

### 3-{[(3-Oxo-3,4-dihydro-2H-benzo[1,4]thiazin-6-ylmethyl)-amino]-methyl}-cyclohexanecarboxylic acid (6-methoxyquinolin-4-yl)-amide

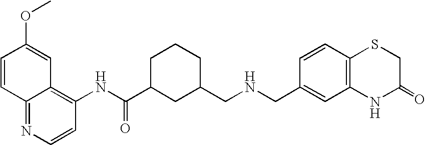

The compound was prepared as in example 1k from aldehyde (6b).

¹H NMR (300 MHz, d₆-DMSO): δ: 10.52 (s, 1H), 9.99 (s, 1H), 8.63 (d, 1H), 8.00 (d, 1H), 7.91 (d, 1H), 7.61 (d, 1H), 7.42 (dd, 1H), 7.26 (d, 1H), 7.01-6.93 (m, 2H), 3.95 (s, 3H), 3.66 (s, 2H), 3.44 (s, 2H), 2.84-2.68 (m, 1H), 2.48-2.37 (m, 2H), 2.11-1.98 (m, 1H), 1.97-1.89 (m, 1H), 1.88-1.75 (m, 2H), 1.65-1.52 (m, 1H), 1.48-1.28 (m, 2H), 1.26-1.07 (m, 2H), 0.99-0.82 (m, 1H)

### Example 50

### 3-{[(Benzo[1,3]dioxol-5-ylmethyl)-amino]-methyl}-cyclohexanecarboxylic acid (6-methoxy-quinolin-4-yl)-amide

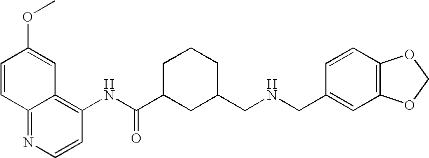

The compound was prepared as in example 1k from benzo[1,3]dioxole-5-carbaldehyde.

¹H NMR (300 MHz, d₆-DMSO): δ: 10.06 (s, 1H), 8.62 (d, 1H), 7.99 (d, 1H), 7.90 (d, 1H), 7.65 (d, 1H), 7.41 (dd, 1H), 7.00 (s, 1H), 6.89-6.82 (m, 2H), 5.99 (s, 2H), 3.95 (s, 3H), 3.71 (s, 2H), 2.93-2.75 (m, 1H), 2.50-2.44 (m, 2H), 2.12-2.00 (m, 1H), 1.98-1.90 (m, 1H), 1.88-1.76 (m, 2H), 1.74-1.58 (m, 1H), 1.49-1.30 (m, 2H), 1.26-1.08 (m, 2H), 1.00-0.82 (m, 1H)

### Example 51

### 3-{[(2,3-Dihydro-benzo[1,4]dioxin-6-ylmethyl)-amino]-methyl}-cyclohexanecarboxylic acid (6-methoxy-quinolin-4-yl)-amide

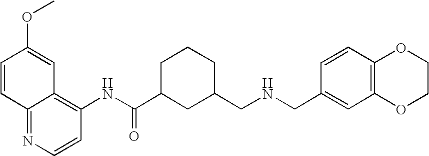

The compound was prepared as in example 1k from 2,3-dihydro-benzo[1,4]dioxine-6-carbaldehyde.

¹H NMR (300 MHz, d₆-DMSO): δ: 10.11 (s, 1H), 8.64 (d, 1H), 7.99 (d, 1H), 7.90 (d, 1H), 7.65 (d, 1H), 7.42 (dd, 1H), 7.00 (d, 1H), 6.94-6.82 (m, 2H), 4.24 (s, 4H), 3.95 (s, 3H), 3.85 (s, 2H), 2.90-2.74 (m, 1H), 2.71-2.55 (m, 2H), 2.12-1.90 (m, 2H), 1.88-1.74 (m, 2H), 1.48-1.12 (m, 5H), 1.04-0.86 (m, 1H)

### Example 52

### 3-{[(Benzo[1,2,5]thiadiazol-5-ylmethyl)-amino]-methyl}-cyclohexanecarboxylic acid (6-methoxy-quinolin-4-yl)-amide

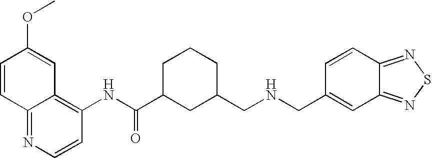

The compound was prepared as in example 1k from benzo[1,2,5]thiadiazole-5-carbaldehyde.

¹H NMR (300 MHz, d₆-DMSO): δ: 9.75 (s, 1H), 8.38 (d, 1H), 7.84-7.71 (m, 3H), 7.66 (d, 1H), 7.48 (dd, 1H), 7.36 (d, 1H), 7.15 (dd, 1H), 3.68 (s, 3H), 3.08 (bs, 1H), 2.60-2.44 (m, 1H), 2.26-2.14 (m, 2H), 1.90-1.78 (m, 1H), 1.74-1.65 (m, 1H), 1.63-1.50 (m, 2H), 1.44-1.28 (m, 1H), 1.26-1.05 (m, 2H), 1.02-0.84 (m, 2H), 0.76-0.55 (m, 2H)

### Example 53

### 3-[((E)-3-Phenyl-allylamino)-methyl]-cyclohexanecarboxylic acid (6-methoxy-quinolin-4-yl)-amide

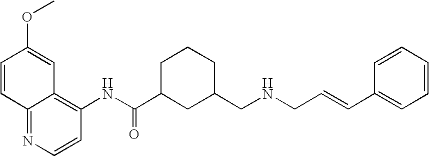

The compound was prepared as in example 1k from cinnamic aldehyde.

¹H NMR (300 MHz, d₆-DMSO): δ: 10.04 (s, 1H), 8.64 (d, 1H), 8.00 (d, 1H), 7.90 (d, 1H), 7.64 (d, 1H), 7.48-7.40 (m, 2H), 7.38-7.30 (m, 2H), 7.28-7.19 (m, 1H), 6.60 (d, 1H), 6.48-6.24 (m, 1H), 3.96 (s, 3H), 3.48-3.40 (m, 2H), 3.34 (bs, 1H), 2.86-2.71 (m, 1H), 2.62-2.54 (m, 2H), 2.10-2.02 (m, 1H), 2.00-1.90 (m, 1H), 1.89-1.76 (m, 2H), 1.74-1.58 (m, 1H), 1.52-1.30 (m, 2H), 1.26-1.10 (m, 2H), 1.06-0.85 (m, 1H)

### Example 54

### 3-{[(Benzo[1,2,5]oxadiazol-5-ylmethyl)-amino]-methyl}-cyclohexanecarboxylic acid (6-methoxy-quinolin-4-yl)-amide

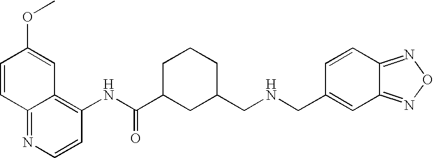

The compound was prepared as in example 1k from benzo[1,2,5]oxadiazole-5-carbaldehyde.

¹H NMR (300 MHz, d₆-DMSO): δ: 9.98 (s, 1H), 8.62 (d, 1H), 8.00-7.97 (m, 2H), 7.94-7.87 (m, 2H), 7.62-7.59 (m, 2H), 7.42 (dd, 1H), 3.94 (s, 3H), 3.83 (s, 2H), 2.80-2.72 (m, 1H), 2.50-2.44 (m, 2H), 2.14-2.02 (m, 1H), 1.98-1.90 (m, 1H), 1.89-1.78 (m, 2H), 1.70-1.52 (m, 1H), 1.50-1.30 (m, 2H), 1.26-1.08 (m, 1H), 1.02-0.84 (m, 1H)

### Example 55

### 3-{[(7-Fluoro-3-oxo-3,4-dihydro-2H-benzo[1,4]thiazin-6-ylmethyl)-amino]-methyl}-cyclohexanecarboxylic acid (6-methoxy-quinolin-4-yl)-amide

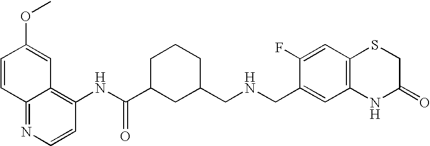

The compound was prepared as in example 1k from aldehyde (24g).

¹H NMR (300 MHz, d₆-DMSO): δ: 10.61 (s, 1H), 10.00 (s, 1H), 8.60 (d, 1H), 7.97 (d, 1H), 7.87 (d, 1H), 7.61 (d, 1H), 7.39 (dd, 1H), 7.22 (d, 1H), 7.12 (d, 1H), 3.92 (s, 3H), 3.91-3.89 (m, 1H), 3.83 (s, 2H), 3.44 (s, 2H), 2.83-2.68 (m, 1H), 2.59-2.48 (m, 1H), 2.06-1.76 (m, 4H), 1.74-1.54 (m, 1H), 1.48-1.08 (m, 4H), 1.03-0.79 (m, 1H)

### Example 56

### 6-({8-[2-(3-Chloro-6-methoxy-quinolin-4-yl)-2-hydroxy-ethyl]-8-aza-bicyclo[3.2.1]oct-3-ylamino}-methyl)-4H-benzo[1,4]oxazin-3-one (enantiomer 1)

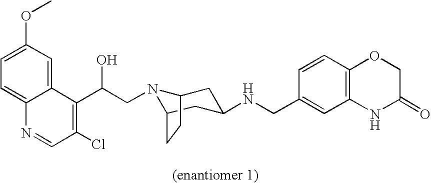

### 56a) 3-Chloro-6-methoxy-quinolin-4-ol

6-Methoxy-quinolin-4-ol (48a) (21.7 g) was dissolved in acetic acid (880 ml), N-chlorosuccinimide (18.2 g) was added and the mixture heated at 60° C. for 4.5 hours, then cooled and evaporated. Excess saturated sodium bicarbonate solution was added and the solid collected and washed with water. The solid was dried in vacuo at 40° C. over night to give the desired product (23.6 g).

¹H NMR (300 MHz, d₆-DMSO): δ: 12.29 (bs, 1H), 8.35 (d, 1H), 7.59-7.52 (m, 2H), 7.33 (dd, 1H), 3.84 (s, 3H)

### 56b) Trifluoro-methanesulfonic acid 3-chloro-6-methoxy-quinolin-4-yl ester

Chloroquinolinol (56a) (3.0 g) was suspended in dichloromethane (50 ml) and cooled to 0° C. Then 2,6-lutidine (2.3 ml), DMAP (270 mg) and trifluoromethanesulfonic acid anhydride (2.4 ml) were added and the mixture was stirred at this temperature for 4 hours. The mixture was diluted with saturated ammonium chloride solution and extracted twice with dichloromethane. The combined organic layers were washed with brine, dried over magnesium sulfate, filtered and evaporated. The residue was purified by flash chromatography (silica gel, ethyl acetate/hexane 2:8) to give the desired product (4.13 g).

¹H NMR (300 MHz, CDCl₃): δ: 8.71 (s, 1H), 7.98 (d, 1H), 7.37 (dd, 1H), 7.21 (d, 1H), 3.89 (s, 3H)

### 56c) 3-Chloro-6-methoxy-4-vinyl-quinoline

Triflate (56b) (3.0 g) and tributylvinylstannane (2.8 ml) were dissolved in dry DMF (60 ml) and degassed by bubbling argon through for 25 minutes. Then PdCl₂(PPh₃)₂ (308 mg) was added and the mixture stirred at 90° C. for 4 hours. The mixture was cooled and concentrated. The residue was dissolved in diethyl ether and washed with water, saturated potassium fluoride solution and brine. The organic layer was dried over magnesium sulfate, filtered and evaporated. The residue was purified by flash chromatography (silica gel, hexane, ethyl acetate/hexane 1:5, 1:1) to give the desired product (1.45 g).

¹H NMR (300 MHz, CDCl₃): δ: 8.60 (s, 1H), 7.94 (d, 1H), 7.34-7.25 (m, 2H), 6.89 (dd, 1H), 5.90 (dd, 1H), 5.72 (dd, 1H), 3.84 (s, 3H)

### 56d) 1-(3-Chloro-6-methoxy-quinolin-4-yl)-ethane-1,2-diol (enantiomer 1)

Vinylquinoline (56c) (470 mg) was dissolved in water (16 ml) and tert-butanol (16 ml), treated with AD mix beta (4.5 g) and stirred at 0° C. for 2 days (freezer). The mixture was treated with sodium metabisulfite (3.3 g) at 0° C., stirred for 60 minutes at this temperature and then filtered. The filtrate was evaporated, the residue taken up with water and extracted twice with ethyl acetate. The combined organic layers were washed with brine, dried over magnesium sulfate, filtered and concentrated. The residue was purified by flash chromatography (silica gel, ethyl acetate) to give the desired product (458 mg).

¹H NMR (300 MHz, d₆-DMSO): δ: 8.65 (s, 1H), 8.29 (d, 1H), 7.95 (d, 1H), 7.42 (dd, 1H), 6.10 (d, 1H), 5.55 (m, 1H), 5.03 (m, 1H), 3.95-3.84 (m, 1H), 3.88 (s, 3H), 3.76-3.65 (m, 1H)

### 56e) Toluene-4-sulfonic acid 2-(3-chloro-6-methoxy-quinolin-4-yl)-2-hydroxy-ethyl ester (enantiomer 1)

Quinolinediol (56d) (386 mg) was suspended in dichloromethane (15 ml), triethylamine (1.1 ml) and THF (3.7 ml). DMAP (28 mg) was added and the mixture cooled with an acetone/dry ice bath and stirred for 5 minutes. Then 4-toluene sulfonyl chloride (290 mg) was added and the mixture stirred for 2.5 hours at this temperature and then kept in the freezer over night. The mixture was diluted with dichloromethane and washed with water and brine. The organic layer was dried over magnesium sulfate, filtered and concentrated (max. 30° C. water bath temperature). The crude product was used for the next step without purification.

### 56f) 3-Chloro-6-methoxy-4-oxiranyl-quinoline (enantiomer 1)

Crude tosylate (56e) (700 mg) was dissolved in DMF (10 ml), cooled with an ice bath, stirred at this temperature for 10 minutes and then treated with sodium hydride (80 mg). The mixture was stirred for 5 minutes at 0° C., then 90 minutes at room temperature, diluted with diethyl ether and washed with water and brine. The organic layer was dried over magnesium sulfate, filtrated and concentrated. The residue was purified by flash chromatography (silica gel, ethyl acetate/hexane 3:7, 1:1) to give the desired product (281 mg).

¹H NMR (300 MHz, CDCl₃): δ: 8.58 (s, 1H), 7.95 (d, 1H), 7.64 (d, 1H), 7.30 (dd, 1H), 4.24 (m, 1H), 3.91 (s, 3H), 3.33 (m, 1H), 2.95 (m, 1H)

### 56g) {8-[2-(3-Chloro-6-methoxy-quinolin-4-yl)-2-hydroxy-ethyl]-8-aza-bicyclo[3.2.1]oct-3-yl}-carbamic acid tert-butyl ester (enantiomer 1)

Epoxide (56f) (273 mg) and amine (8g) (262 mg) were dissolved in DMF (10 ml), treated with potassium carbonate (160 mg) and lithium perchlorate (129 mg) and stirred at 140° C. over night. The mixture was concentrated, dissolved in dichloromethane/methanol 9:1 and washed with water. The organic layer was dried over magnesium sulfate, filtered and concentrated. The residue was purified by flash chromatography (silica gel, dichloromethane/methanol 19:1, 9:1), to give the desired product (442 mg).

¹H NMR (300 MHz, d₆-DMSO): δ: 8.64 (s, 1H), 8.16 (d, 1H), 7.93 (d, 1H), 7.42 (dd, 1H), 6.63 (d, 1H), 5.93 (bs, 1H), 5.57 (m, 1H), 3.89 (s, 3H), 3.60-3.43 (m, 1H), 3.35-3.25 (m, 1H), 3.12-2.95 (m, 2H), 2.79-2.67 (m, 1H), 1.95-1.76 (m, 3H), 1.59-1.36 (m, 5H), 1.35 (s, 9H)

### 56h) 2-(3-Amino-8-aza-bicyclo[3.2.1]oct-8-yl)-1-(3-chloro-6-methoxy-quinolin-4-yl)-ethanol (enantiomer 1)

Boc-amine (56g) (435 mg) was dissolved in dichloromethane (20 ml), treated with TFA (0.072 ml) and stirred at room temperature over night. The mixture was made alkaline with 2N sodium hydroxide solution and the layers were separated. The aqueous layer was back extracted with dichloromethane. The combined organic layers were washed with water and brine, dried over magnesium sulfate, filtered and concentrated. The residue was purified by flash chromatography (silica gel, dichloromethane/(methanol/ammonia 9:1) 9:1) to give the desired product (232 mg).

¹H NMR (300 MHz, CDCl₃): δ: 8.51 (s, 1H), 8.12-8.01 (m, 1H), 7.92-7.85 (m, 1H), 7.31-7.23 (m, 1H), 5.62-5.58 (m, 1H), 3.85 (s, 3H), 3.73-3.56 (m, 1H), 3.54-3.46 (m, 1H), 3.46-3.19 (m, 2H), 2.83-2.58 (m, 2H), 2.05-1.72 (m, 7H), 1.70-1.52 (m, 3H)

### 56i) Title Compound

The compound was prepared as in example 1k from aldehyde (1j).

¹H NMR (300 MHz, d₆-DMSO): δ: 8.43 (s, 1H), 7.95 (d, 1H), 7.72 (d, 1H), 7.21 (dd, 1H), 6.67-6.54 (m, 3H), 5.70 (bs, 1H), 5.35 (m, 1H), 4.32 (s, 2H), 3.67 (s, 3H), 3.19-3.03 (m, 3H), 2.91-2.72 (m, 2H), 2.60-2.36 (m, 2H), 1.68-1.46 (m, 4H), 1.44-0.90 (m, 6H)

### Example 57

### 6-({8-[2-(3-Chloro-6-methoxy-quinolin-4-yl)-2-hydroxy-ethyl]-8-aza-bicyclo[3.2.1]oct-3-ylamino}-methyl)-4H-benzo[1,4]thiazin-3-one (enantiomer 1)

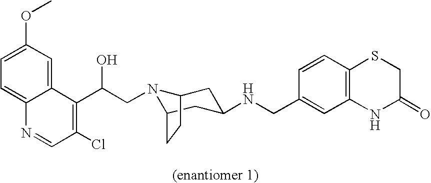

The compound was prepared as in example 1k from aldehyde (6b).

¹H NMR (300 MHz, d₆-DMSO): δ: 10.50 (s, 1H), 8.65 (s, 1H), 8.17 (d, 1H), 7.94 (d, 1H), 7.42 (dd, 1H), 7.21 (d, 1H), 6.93-6.89 (m, 2H), 5.91 (bs, 1H), 5.57 (m, 1H), 4.13-4.05 (m, 3H), 3.89 (s, 3H), 3.59 (s, 2H), 3.35-3.26 (m, 1H), 3.11-2.93 (m, 2H), 2.82-2.60 (m, 2H), 1.90-1.53 (m, 2H), 1.50-1.18 (m, 4H)

### Example 58

### 2-{3-[(Benzo[1,2,5]thiadiazol-5-ylmethyl)-amino]-8-aza-bicyclo[3.2.1]oct-8-yl}-1-(3-chloro-6-methoxy-quinolin-4-yl)-ethanol (enantiomer 1)

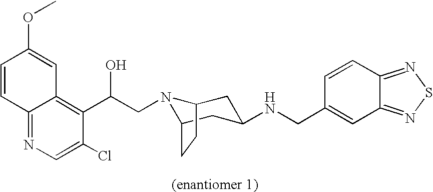

The compound was prepared as in example 1k from benzo[1,2,5]thiadiazole-5-carbaldehyde.

¹H NMR (300 MHz, CDCl₃): δ: 8.47 (s, 1H), 8.10 (d, 1H), 7.87-7.79 (m, 3H), 7.51-7.43 (m, 1H), 5.64 (d, 1H), 3.86 (s, 2H), 3.81 (s, 3H), 3.60-3.48 (m, 1H), 3.44-3.35 (m, 1H), 2.92-2.76 (m, 2H), 2.74-2.65 (m, 1H), 2.00-1.70 (m, 6H), 1.68-1.48 (m, 3H)

### Example 59

### 2-{3-[(Benzo[1,2,5]oxadiazol-5-ylmethyl)-amino]-8-aza-bicyclo[3.2.1]oct-8-yl}-1-(3-chloro-6-methoxy-quinolin-4-yl)-ethanol (enantiomer 1)

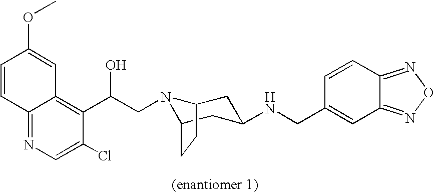

The compound was prepared as in example 1k from benzo[1,2,5]oxadiazole-5-carbaldehyde.

¹H NMR (300 MHz, CDCl₃): δ: 8.51 (s, 1H), 8.15 (d, 1H), 7.87 (d, 1H), 7.72-7.64 (m, 3H), 7.34 (dd, 1H), 7.27 (dd, 1H), 5.66 (m, 1H), 4.71 (s, 1H), 3.86 (s, 3H), 3.81 (s, 2H), 3.60-3.50 (m, 1H), 3.55-3.45 (m, 1H), 2.92-2.66 (m, 3H), 2.06-1.51 (m, 8H)

### Example 60

### 6-({8-[2-(3-Chloro-6-methoxy-quinolin-4-yl)-2-hydroxy-ethyl]-8-aza-bicyclo[3.2.1]oct-3-ylamino}-methyl)-7-fluoro-4H-benzo[1,4]thiazin-3-one (enantiomer 1)

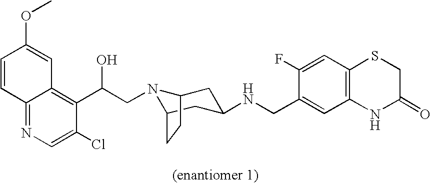

The compound was prepared as in example 1k from aldehyde (24g).

¹H NMR (300 MHz, d₆-DMSO): δ: 10.51 (s, 1H), 8.65 (s, 1H), 8.17 (d, 1H), 7.93 (d, 1H), 7.42 (dd, 1H), 7.17-7.03 (m, 2H), 5.90 (m, 1H), 5.76 (s, 1H), 5.56 (m, 1H), 4.46 (m, 1H), 3.89 (s, 3H), 3.59 (s, 2H), 3.44 (s, 2H), 3.12-2.95 (m, 2H), 2.83-2.72 (m, 1H), 2.70-2.56 (m, 1H), 1.85-1.72 (m, 2H), 1.70-1.54 (m, 2H), 1.50-1.31 (m, 3H), 1.28-1.13 (m, 1H)

### Example 61

### 2-{3-[(Benzo[1,3]dioxol-5-ylmethyl)-amino]-8-aza-bicyclo[3.2.1]oct-8-yl}-1-(3-chloro-6-methoxy-quinolin-4-yl)-ethanol (enantiomer 1)

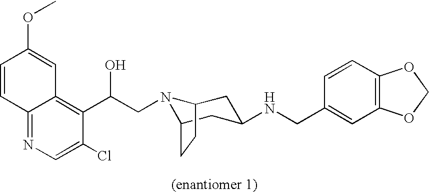

The compound was prepared as in example 1k from benzo[1,3]dioxole-5-carbaldehyde.

¹H NMR (300 MHz, d₆-DMSO): δ: 8.65 (s, 1H), 8.16 (d, 1H), 7.93 (d, 1H), 7.42 (dd, 1H), 6.87-6.72 (m, 3H), 5.96 (s, 1H), 5.95-5.84 (m, 1H), 5.60-5.52 (m, 1H), 3.89 (s, 3H), 3.54 (s, 2H), 3.35-3.20 (m, 1H), 3.11-2.92 (m, 2H), 2.83-2.72 (m, 1H), 2.70-2.55 (m, 1H), 1.90-1.70 (m, 2H), 1.68-1.50 (m, 2H), 1.46-1.15 (m, 5H)

### Example 62

### 1-(3-Chloro-6-methoxy-quinolin-4-yl)-2-{3-[(2,3-dihydro-benzo[1,4]dioxin-6-ylmethyl)-amino]-8-aza-bicyclo[3.2.1]oct-8-yl}-ethanol (enantiomer 1)

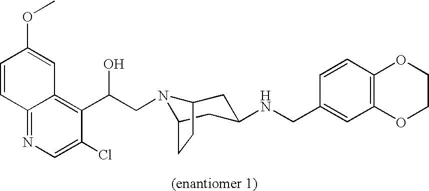

The compound was prepared as in example 1k from 2,3-dihydro-benzo[1,4]dioxine-6-carbaldehyde.

¹H NMR (300 MHz, d₆-DMSO): δ: 8.64 (s, 1H), 8.17 (d, 1H), 7.93 (d, 1H), 7.42 (dd, 1H), 6.80-6.72 (m, 3H), 5.89 (d, 1H), 5.59-5.55 (m, 1H), 4.21 (s, 4H), 3.89 (s, 3H), 3.49 (s, 2H), 3.26 (m, 1H), 3.17 (d, 1H), 3.10-2.93 (m, 2H), 2.80-2.70 (m, 1H), 2.68-2.52 (m, 1H), 1.96-1.50 (m, 4H), 1.46-1.15 (m, 4H)

### Example 63

### 1-(3-Chloro-6-methoxy-quinolin-4-yl)-2-[3-((E)-3-phenyl-allylamino)-8-aza-bicyclo[3.2.1]oct-8-yl]-ethanol (enantiomer 1)

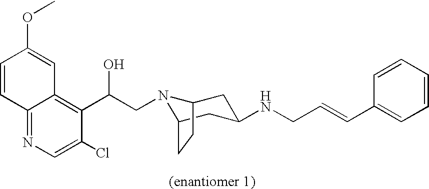

The compound was prepared as in example 1k from cinnamic aldehyde.

¹H NMR (300 MHz, CDCl₃): δ: 8.54 (s, 1H), 8.17 (d, 1H), 7.89 (d, 1H), 7.34-7.15 (m, 6H), 6.58-6.51 (m, 1H), 6.35-6.23 (m, 1H), 5.62-5.58 (m, 1H), 3.87 (s, 3H), 3.56-3.27 (m, 4H), 3.14-2.98 (m, 1H), 2.83-2.65 (m, 2H), 2.10-1.66 (m, 8H), 1.64-1.24 (m, 2H)

### Example 64

### 6-[({1-[2-(3-Chloro-6-methoxy-quinolin-4-yl)-2-hydroxy-ethyl]-piperidin-3-ylmethyl}-amino)-methyl]-4H-benzo[1,4]oxazin-3-one (enantiomer 1)

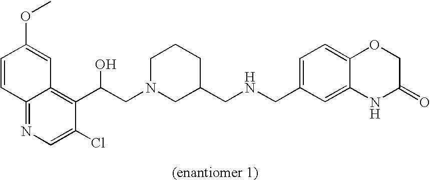

### 64a) {1-[2-(3-Chloro-6-methoxy-quinolin-4-yl)-2-hydroxy-ethyl]-piperidin-3-ylmethyl}-carbamic acid tert-butyl ester (enantiomer 1)

Epoxide (56f) (900 mg), 3-(N-Boc-aminomethyl)piperidine (819 mg), potassium carbonate (555 mg) and lithium perchlorate (405 mg) were suspended in DMF (9 ml) and heated in the microwave for 35 minutes at 130° C. The mixture was concentrated, the residue dissolved in ethyl acetate and washed with water and brine. The organic layer was dried over magnesium sulfate, filtered and evaporated. The residue was purified by flash chromatography (silica gel, dichloromethane/methanol 97:3) to give the desired product (1.6 g).

MS (EI): m/z: 450 [M+H]⁺

### 64b) 2-(3-Aminomethyl-piperidin-1-yl)-1-(3-chloro-6-methoxyquinolin-4-yl)-ethanol (enantiomer 1)

Boc-amine (64a) (1.60 g) was dissolved in dichloromethane (27 ml), treated with TFA (2.7 ml) at 0-5° C. and stirred at room temperature over night. The mixture was made alkaline with 2N sodium hydroxide solution and the layers were separated. The aqueous layer was extracted once with dichloromethane. The combined organic layers were washed with brine, dried over magnesium sulfate, filtered and concentrated. The residue was purified by flash chromatography (silica gel, dichloromethane/methanol 9:1+1% ammonia) to give the desired product (995 mg).

¹H NMR (300 MHz, d₆-DMSO): δ: 8.65 (s, 1H), 8.15 (m, 1H), 7.92 (d, 1H), 7.42 (dd, 1H), 5.69-5.65 (m, 1H), 3.89 (s, 3H), 3.33 (bs, 2H), 3.06-2.92 (m, 2H), 2.85-2.58 (m, 2H), 2.43-2.26 (m, 2H), 2.10-1.96 (m, 1H), 1.91-1.25 (m, 6H), 0.92-0.81 (m, 1H)

### 64c) Title Compound

The compound was prepared as in example 1k from aldehyde (1j).

¹H NMR (300 MHz, d₆-DMSO): δ: 10.63 (s, 1H), 8.63 (d, 1H), 8.14 (d, 1H), 7.92 (d, 1H), 7.43 (dd, 1H), 6.94-6.75 (m, 3H), 5.98-5.86 (m, 1H), 5.76 (s, 1H), 5.70-5.60 (m, 1H), 4.54 (d, 2H), 3.89 (s, 3H), 3.52 (d, 2H), 3.08-2.83 (m, 2H), 2.74-2.59 (m, 1H), 2.35-2.16 (m, 2H), 2.14-1.95 (m, 2H), 1.85-1.72 (m, 1H), 1.70-1.23 (m, 5H), 0.92-0.75 (m, 1H)

### Example 65

### 2-(3-{[(Benzo[1,3]dioxol-5-ylmethyl)-amino]-methyl}-piperidin-1-yl)-1-(3-chloro-6-methoxy-quinolin-4-yl)-ethanol (enantiomer 1)

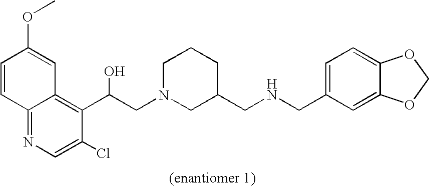

The compound was prepared as in example 1k from benzo[1,3]dioxole-5-carbaldehyde.

¹H NMR (300 MHz, d₆-DMSO): δ: 8.64 (d, 1H), 8.16 (d, 1H), 7.94 (d, 1H), 7.43 (dd, 1H), 6.93-6.68 (m, 3H), 5.96 (d, 2H), 5.94-5.88 (m, 1H), 5.72-5.62 (m, 1H), 3.87 (s, 3H), 3.52 (d, 2H), 3.09-2.82 (m, 2H), 2.72-2.58 (m, 2H), 2.35-2.18 (m, 2H), 2.16-1.92 (m, 2H), 1.94-1.71 (m, 1H), 1.69-1.35 (m, 4H), 0.95-0.75 (m, 1H)

### Example 66

### 1-(3-Chloro-6-methoxy-quinolin-4-yl)-2-(3-{[(2,3-dihydro-benzo[1,4]dioxin-6-ylmethyl)-amino]-methyl}-piperidin-1-yl)-ethanol (enantiomer 1)

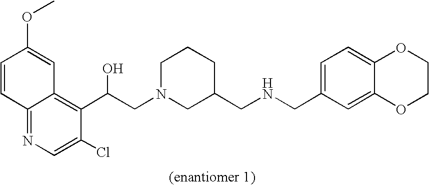

The compound was prepared as in example 1k from 2,3-dihydro-benzo[1,4]dioxine-6-carbaldehyde.

¹H NMR (300 MHz, d₆-DMSO): δ: 8.64 (d, 1H), 8.14 (d, 1H), 7.92 (d, 1H), 7.41 (dd, 1H), 6.82-6.66 (m, 3H), 5.91 (d, 1H), 5.72-5.61 (m, 1H), 4.22 (d, 2H), 3.88 (s, 3H), 3.49 (d, 2H), 3.08-2.83 (m, 2H), 2.74-2.60 (m, 2H), 2.38-2.18 (m, 2H), 2.16-1.96 (m, 1H), 1.90-1.74 (m, 2H), 1.70-1.25 (m, 4H), 0.95-0.76 (m, 1H)

### Example 67

### 6-[({1-[2-(3-Chloro-6-methoxy-quinolin-4-yl)-2-hydroxy-ethyl]-piperidin-3-ylmethyl}-amino)-methyl]-7-fluoro-4H-benzo[1,4]thiazin-3-one (enantiomer 1)

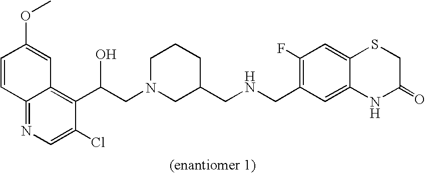

The compound was prepared as in example 1k from aldehyde (24g).

¹H NMR (300 MHz, d₆-DMSO): δ: 10.53 (d, 1H), 8.63 (d, 1H), 8.15 (d, 1H), 7.92 (dd, 1H), 7.44-7.39 (m, 1H), 7.17 (dd, 1H), 7.03 (dd, 1H), 5.91 (d, 1H), 5.76 (s, 1H), 5.74-5.62 (m, 1H), 3.88 (s, 3H), 3.58 (d, 2H), 3.45 (d, 2H), 3.05-2.90 (m, 2H), 2.75-2.60 (m, 2H), 2.39-2.22 (m, 2H), 2.14-1.98 (m, 1H), 1.87-1.76 (m, 1H), 1.74-1.24 (m, 4H), 0.96-0.78 (m, 1H)

### Example 68

### 6-[({1-[2-(3-Chloro-6-methoxy-quinolin-4-yl)-2-hydroxy-ethyl]-piperidin-3-ylmethyl}-amino)-methyl]-4H-benzo[1,4]thiazin-3-one (enantiomer 1)

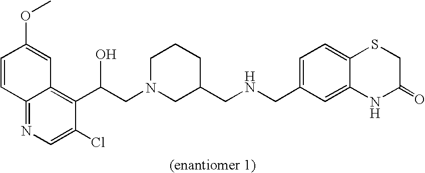

The compound was prepared as in example 1k from aldehyde (6b).

¹H NMR (300 MHz, d₆-DMSO): δ: 10.49 (d, 1H), 8.63 (d, 1H), 8.15 (d, 1H), 7.93 (d, 1H), 7.44-7.40 (m, 1H), 7.26-7.20 (m, 1H), 6.97-6.88 (m, 2H), 5.96-5.85 (m, 1H), 5.70-5.62 (m, 1H), 3.88 (s, 3H), 3.54 (d, 2H), 3.43 (d, 2H), 3.06-2.90 (m, 2H), 2.76-2.58 (m, 2H), 2.38-2.19 (m, 2H), 2.15-1.88 (m, 2H), 1.86-1.73 (m, 1H), 1.72-1.21 (m, 5H)

### Example 69

### 1-(3-Chloro-6-methoxy-quinolin-4-yl)-2-{3-[((E)-3-phenyl-allylamino)-methyl]-piperidin-1-yl}-ethanol (enantiomer 1)

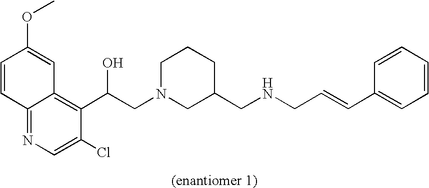

The compound was prepared as in example 1k from cinnamic aldehyde.

¹H NMR (300 MHz, d₆-DMSO): δ: 8.64 (s, 1H), 8.15 (d, 1H), 7.93 (d, 1H), 7.44-7.19 (m, 6H), 6.52-6.44 (m, 1H), 6.32-6.21 (m, 1H), 5.98-5.88 (m, 1H), 5.71-5.62 (m, 1H), 3.88 (s, 3H), 3.28-3.18 (m, 2H), 3.08-2.90 (m, 2H), 2.76-2.59 (m, 2H), 2.43-2.23 (m, 2H), 2.16-1.98 (m, 1H), 1.85-1.22 (m, 6H), 0.98-0.75 (m, 1H)

### Example 70

### 2-(3-{[(Benzo[1,2,5]thiadiazol-5-ylmethyl)-amino]-methyl}-piperidin-1-yl)-1-(3-chloro-6-methoxy-quinolin-4-yl)-ethanol (enantiomer 1)

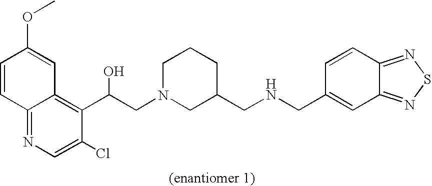

The compound was prepared as in example 1k from benzo[1,2,5]thiadiazole-5-carbaldehyde.

¹H NMR (300 MHz, d₆-DMSO): δ: 8.64 (s, 1H), 8.15 (s, 1H), 8.08-7.85 (m, 3H), 7.75-7.60 (m, 1H), 7.57-7.35 (m, 1H), 5.99-5.86 (m, 1H), 5.74-5.62 (m, 1H), 3.88 (s, 3H), 3.83 (s, 2H), 3.12-2.82 (m, 2H), 2.76-2.58 (m, 2H), 2.44-2.22 (m, 3H), 2.19-1.98 (m, 1H), 1.90-1.21 (m, 5H), 1.03-0.78 (m, 1H)

### Example 71

### 6-[({4-[2-(3-Chloro-6-methoxy-quinolin-4-yl)-2-hydroxy-ethyl]-morpholin-2-ylmethyl}-amino)-methyl]-4H-benzo[1,4]oxazin-3-one (enantiomer 1)

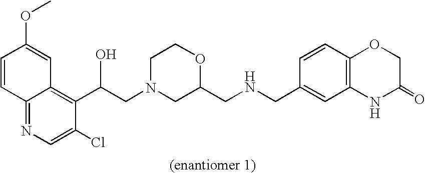

### 71a) {4-[2-(3-Chloro-6-methoxy-quinolin-4-yl)-2-hydroxy-ethyl]-morpholin-2-ylmethyl}-carbamic acid tert-butyl ester (enantiomer 1)

Epoxide (56f) (1.00 g) and morpholin-2-ylmethyl-carbamic acid tert-butyl ester (31b) (0.92 g) were dissolved in DMF (13 ml), treated with potassium carbonate (0.62 g) and lithium perchlorate (0.45 g) and stirred at 80° C. over night. The mixture was concentrated, the residue dissolved in dichloromethane and washed with water and brine. The organic layer was dried over magnesium sulfate, filtered and concentrated. The residue was purified by flash chromatography (silica gel, dichloromethane/methanol 97:3) to give the desired product (1.46 g).

¹H NMR (300 MHz, CDCl₃): δ: 8.52 (s, 1H), 8.09 (d, 1H), 7.88 (d, 1H), 7.28 (dd, 1H), 6.99-6.83 (m, 1H), 4.89-4.78 (m, 1H), 3.99-3.90 (m, 1H), 3.87 (s, 3H), 3.41-2.94 (m, 7H), 2.75-2.56 (m, 2H), 2.54-2.18 (m, 2H), 1.38 (s, 9H)

### 71b) 2-(2-Aminomethyl-morpholin-4-yl)-1-(3-chloro-6-methoxyquinolin-4-yl)-ethanol (enantiomer 1)

Boc-amine (71a) (1.46 g) was dissolved in dichloromethane (25 ml), treated with TFA (2.5 ml) and stirred at room temperature over night. The mixture was made alkaline with 2N sodium hydroxide solution and the layers were separated. The aqueous layer was extracted once more with dichloromethane. The combined organic layers were washed with brine, dried over magnesium sulfate, filtered and concentrated. The residue was purified by flash chromatography (silica gel, dichloromethane/methanol 9:1+1% ammonia) to give the desired product (708 mg).

¹H NMR (300 MHz, d₆-DMSO): δ: 8.65 (s, 1H), 8.16 (d, 1H), 7.94 (d, 1H), 7.43 (dd, 1H), 6.02 (bs, 1H), 5.71-5.67 (m, 1H), 3.89 (s, 3H), 3.79-3.70 (m, 1H), 3.51-3.22 (m, 5H), 3.04-2.97 (m, 2H), 2.74-2.45 (m, 3H), 2.27-2.12 (m, 1H), 1.96-1.86 (m, 1H)

### 71c) Title Compound

The compound was prepared as in example 1k from aldehyde (1j).

¹H NMR (300 MHz, d₆-DMSO): δ: 10.79 (d, 1H), 8.65 (s, 1H), 8.16 (d, 1H), 7.94 (d, 1H), 7.43 (dd, 1H), 6.88-6.82 (m, 3H), 6.02 (d, 1H), 5.71-5.66 (m, 1H), 4.54 (d, 2H), 3.89 (s, 3H), 3.84-3.69 (m, 1H), 3.67-3.58 (d, 2H), 3.57-3.29 (m, 4H), 3.09-2.88 (m, 2H), 2.78-2.47 (m, 3H), 2.31-2.12 (m, 1H), 2.02-1.87 (m, 1H)

### Example 72

### 2-(2-{[(Benzo[1,3]dioxol-5-ylmethyl)-amino]-methyl}-morpholin-4-yl)-1-(3-chloro-6-methoxy-quinolin-4-yl)-ethanol (enantiomer 1)

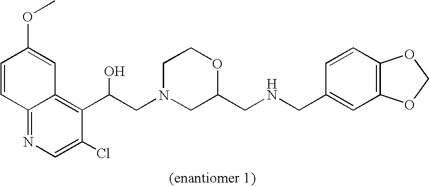

The compound was prepared as in example 1k from benzo[1,3]dioxole-5-carbaldehyde.

¹H NMR (300 MHz, d₆-DMSO): δ: 8.65 (s, 1H), 8.15 (d, 1H), 7.95 (d, 1H), 7.44 (dd, 1H), 6.92-6.68 (m, 3H), 6.02 (d, 1H), 5.97 (d, 2H), 5.74-5.63 (m, 1H), 3.90 (s, 3H), 3.81-3.66 (m, 1H), 3.62-3.54 (d, 2H), 3.53-3.35 (m, 2H), 3.10-2.86 (m, 2H), 2.79-2.54 (m, 3H), 2.48-2.33 (m, 2H), 2.30-2.13 (m, 1H), 2.02-1.87 (m, 1H)

### Example 73

### 1-(3-Chloro-6-methoxy-quinolin-4-yl)-2-(2-{[(2,3-dihydro-benzo[1,4]dioxin-6-ylmethyl)-amino]-methyl}-morpholin-4-yl)-ethanol (enantiomer 1)

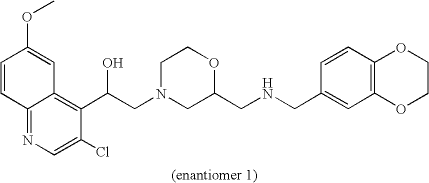

The compound was prepared as in example 1k from 2,3-dihydro-benzo[1,4]dioxine-6-carbaldehyde.

¹H NMR (300 MHz, d₆-DMSO): δ: 8.65 (s, 1H), 8.15 (d, 1H), 7.94 (d, 1H), 7.43 (dd, 1H), 6.81-6.69 (m, 3H), 6.01 (d, 1H), 5.71-5.66 (m, 1H), 4.21 (d, 4H), 3.89 (s, 3H), 3.81-3.65 (m, 1H), 3.59-3.52 (d, 2H), 3.49-3.35 (m, 2H), 3.08-2.86 (m, 2H), 2.79-2.55 (m, 3H), 2.50-2.34 (2H), 2.27-2.13 (m, 1H), 2.02-1.87 (m, 1H)

### Example 74

### 2-(2-{[(Benzo[1,2,5]thiadiazol-5-ylmethyl)-amino]-methyl}-morpholin-4-yl)-1-(3-chloro-6-methoxy-quinolin-4-yl)-ethanol (enantiomer 1)

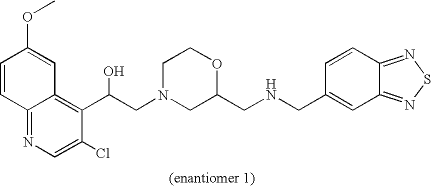

The compound was prepared as in example 1k from benzo[1,2,5]thiadiazole-5-carbaldehyde.

¹H NMR (300 MHz, d₆-DMSO): δ: 8.64 (d, 1H), 8.16 (d, 1H), 8.05-7.92 (m, 3H), 7.73-7.66 (m, 1H), 7.42 (dd, 1H), 6.01 (d, 1H), 5.72-5.66 (m, 1H), 3.89 (d, 2H), 3.88 (s, 3H), 3.83-3.68 (m, 1H), 3.63-3.37 (m, 2H), 3.12-2.88 (m, 2H), 2.84-2.45 (m, 5H), 2.30-2.14 (m, 1H), 2.04-1.90 (m, 1H)

### Example 75

### 6-[({4-[2-(3-Chloro-6-methoxy-quinolin-4-yl)-2-hydroxy-ethyl]-morpholin-2-ylmethyl}-amino)-methyl]-7-fluoro-4H-benzo[1,4]thiazin-3-one (enantiomer 1)

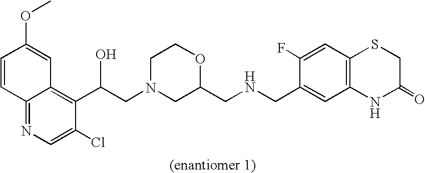

The compound was prepared as in example 1k from aldehyde (24g).

¹H NMR (300 MHz, d₆-DMSO): δ: 10.53 (d, 1H), 8.65 (s, 1H), 8.15 (d, 1H), 7.94 (d, 1H), 7.43 (dd, 1H), 7.18 (dd, 1H), 7.03 (dd, 1H), 6.00 (d, 1H), 5.72-5.66 (m, 1H), 3.89 (s, 3H), 3.84-3.70 (m, 1H), 3.68-3.59 (d, 2H), 3.57-3.35 (m, 4H), 3.07-2.88 (m, 2H), 2.75-2.39 (m, 5H), 2.30-2.13 (m, 1H), 2.04-1.90 (m, 1H)

### Example 76

### 6-[({4-[2-(3-Chloro-6-methoxy-quinolin-4-yl)-2-hydroxy-ethyl]-morpholin-2-ylmethyl}-amino)-methyl]-4H-benzo[1,4]thiazin-3-one (enantiomer 1)

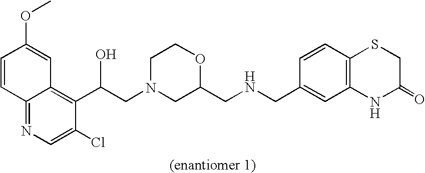

The compound was prepared as in example 1k from aldehyde (6b).

¹H NMR (300 MHz, d₆-DMSO): δ: 10.51 (d, 1H), 8.65 (s, 1H), 8.16 (d, 1H), 7.95 (d, 1H), 7.43 (dd, 1H), 7.23 (dd, 1H), 7.00-6.85 (m, 2H), 6.08-5.95 (m, 1H), 5.75-5.62 (m, 1H), 3.91 (s, 3H), 3.84-3.69 (m, 1H), 3.68-3.56 (d, 2H), 3.55-3.36 (m, 4H), 3.07-2.87 (m, 2H), 2.78-2.36 (m, 5H), 2.33-2.11 (m, 1H), 2.05-1.88 (m, 1H)

### Example 77

### 1-(3-Chloro-6-methoxy-quinolin-4-yl)-2-{2-[((E)-3-phenyl-allylamino)-methyl]-morpholin-4-yl}-ethanol (enantiomer 1)

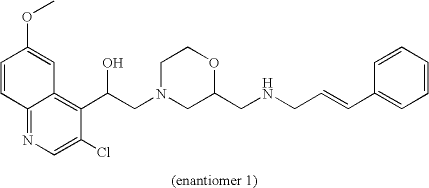

The compound was prepared as in example 1k from cinnamic aldehyde.

¹H NMR (300 MHz, d₆-DMSO): δ: 8.65 (s, 1H), 8.16 (d, 1H), 7.94 (d, 1H), 7.45-7.19 (m, 6H), 6.55-6.46 (m, 1H), 6.34-6.21 (m, 1H), 6.01 (d, 1H), 3.89 (s, 3H), 3.85-3.68 (m, 1H), 3.59-3.25 (m, 4H), 3.10-2.87 (m, 2H), 2.80-2.43 (m, 5H), 2.33-2.14 (m, 1H), 2.04-1.92 (m, 1H)

### Example 78

### 2-{3-[(2,3-Dihydro-benzo[1,4]dioxin-6-ylmethyl)-amino]-8-aza-bicyclo[3.2.1]oct-8-yl}-1-(3-methoxy-quinoxalin-5-yl)-ethanol

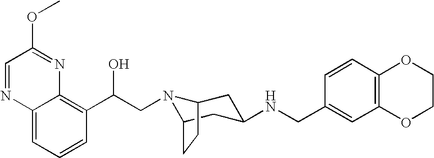

### 78a) 8-Methyl-1H-quinoxalin-2-one

2,3-Diaminotoluene (10.00 g) was dissolved in ethanol (164 ml), treated with ethyl glyoxalate (24.4 ml) and then refluxed for 2 hours. The mixture was cooled to room temperature, the precipitate was filtered off and washed with ethanol and pentane to give the product (11.26 g) as a 3:1 mixture of the regioisomers (desired/undesired).

¹H NMR (300 MHz, CDCl₃): δ: 8.34-8.26 (m, 2H), 7.71-7.65 (m, 1H), 7.43-7.30 (m, 2H), 7.24-7.06 (m, 5H), 2.63 (s, 3H), 2.48 (s, 3H)

### 78b) 2-Methoxy-8-methyl-quinoxaline

Quinoxalinone (78a) (10.25 g) was dissolved in DMF (300 ml), potassium carbonate (8.84 g) and methyl iodide (4 ml) were added and the mixture stirred at room temperature over night. Water (150 ml) was added to the mixture and the aqueous layer was extracted three times with ethyl acetate. The combined organic layers were washed with brine, dried over magnesium sulfate, filtered and evaporated. The crude product was purified by flash chromatography (silica gel, hexane/ethyl acetate 2:1, 1:1) to give the desired product (3.73 g).

¹H NMR (300 MHz, CDCl₃): δ: 8.40 (s, 1H), 7.79 (d, 1H), 7.48-7.36 (m, 2H), 4.04 (s, 3H), 2.62 (s, 3H)

### 78c) 8-Dibromomethyl-2-methoxy-quinoxaline

Quinoxaline (78b) (610 mg) was dissolved in carbon tetrachloride (40 ml), treated with NBS (1.56 g) and AIBN (58 mg). The mixture was refluxed for 4 hours, then diluted with water and extracted with dichloromethane. The organic layer washed once with water and then dried over magnesium sulfate, filtered and concentrated. The residue was triturated with diethyl ether and the precipitate filtered off to give the desired product (1.10 g).

¹H NMR (300 MHz, CDCl₃): δ: 8.46 (s, 1H), 8.23 (dd, 1H), 7.96 (dd, 1H), 7.84 (s, 1H), 7.61-7.56 (m, 1H), 4.09 (s, 3H)

### 78d) 3-Methoxy-quinoxaline-5-carbaldehyde

Dibromoquinoxaline (78c) (1.1 g) was dissolved in ethanol (30 ml) and treated with a solution of silver nitrate (1.13 g) in water (6 ml) at room temperature and stirred over night. The suspension was filtered through Celite®, washed with THF/ethyl:acetate (1:1, 100 ml) and the filtrate was concentrated. The residue was taken up with water and extracted with ethyl acetate. The combined organic layers were washed with brine, dried over magnesium sulfate, filtered and concentrated to give the desired product (604 mg).

¹H NMR (300 MHz, CDCl₃): δ: 11.20 (s, 1H), 8.50 (s, 1H), 8.42 (d, 1H), 7.65-7.55 (m, 1H), 4.10 (s, 3H)

### 78e) 2-Methoxy-8-oxiranyl-quinoxaline

Aldehyde (78d) (600 mg) was suspended in acetonitrile (32 ml) containing 8 drops of water and heated to 60° C. Then trimethyl sulfoniumiodide (670 mg) and potassium hydroxide (1.25 g) were added and the mixture stirred at 60° C. for 2.5 hours. The mixture was filtered and the filtrate evaporated. The residue was taken up with water and extracted with ethyl acetate. The combined organic layers were washed with brine, dried over magnesium sulfate, filtered and concentrated. The residue was purified by flash chromatography (silica gel, dichloromethane) to give the desired product (463 mg).

¹H NMR (300 MHz, CDCl₃): δ: 8.44 (s, 1H), 7.90-7.86 (m, 1H), 7.50-7.44 (m, 1H), 4.85-4.83 (m, 1H), 4.05 (s, 3H), 3.26-3.23 (m, 1H), 2.79-2.76 (m, 1H)

### 78f) {8-[2-Hydroxy-2-(3-methoxy-quinoxalin-5-yl)-ethyl]-8-aza-bicyclo[3.2.1]oct-3-yl}-carbamic acid tert-butyl ester

To a solution of epoxide (78e) (179 mg) and amine (8g) (200 mg) in DMF (5 ml) was added lithium perchlorate (110 mg). The mixture was stirred for 5 days at room temperature. Water (70 ml) was added and the mixture extracted with ethyl acetate. The combined organic layers were dried over magnesium sulfate, filtered and evaporated. The residue was purified by flash chromatography (silica gel, dichloromethane/methanol 19:1) to give the desired product (111 mg).

¹H NMR (300 MHz, CDCl₃): δ: 8.50 (s, 1H), 8.03-7.94 (m, 2H), 7.64-7.58 (m, 1H), 5.80 (bd, 1H), 4.58-4.49 (m, 1H), 4.18 (s, 3H), 3.97-3.82 (m, 1H), 3.77-3.65 (m, 1H), 3.45-3.35 (m, 1H), 3.18-3.07 (m, 1H), 2.53-2.38 (m, 1H), 2.12-1.73 (m, 9H), 1.46 (s, 9H)

### 78g) 2-(3-Amino-8-aza-bicyclo[3.2.1]oct-8-yl)-1-(3-methoxyquinoxalin-5-yl)-ethanol

To a solution of Boc-amine (78f) (111 mg) in dichloromethane (2 ml) was added TFA (1 ml) and the mixture stirred for 20 minutes at room temperature. The volatiles were removed and dichloromethane (5 ml) and 2N sodium hydroxide solution (5 ml) added. The aqueous layer was extracted three times with dichloromethane. The combined organic layers were dried over magnesium sulfate, filtered and evaporated. The residue was purified by flash chromatography (silica gel, dichloromethane/methanol 19:1) to give the desired product (71 mg).

¹H NMR (300 MHz, CDCl₃): δ: 8.40 (s, 1H), 7.88-7.83 (m, 2H), 7.54-7.49 (m, 1H), 5.62-5.58 (m, 1H), 3.98 (s, 3H), 3.52-3.42 (m, 1H), 3.20-3.10 (m, 1H), 3.05-2.89 (m, 3H), 2.29-2.15 (m, 1H), 1.98-1.83 (m, 1H), 1.82-1.66 (m, 3H), 1.62-1.41 (m, 3H), 1.27-1.12 (m, 3H), 0.90-0.71 (m, 1H)

### 78h) Title Compound

The compound was prepared as in example 1k from 2,3-dihydro-benzo[1,4]dioxine-6-carbaldehyde.

MS (EI): m/z: 477 [M+H]⁺

### Example 79

### 6-({8-[2-Hydroxy-2-(3-methoxy-quinoxalin-5-yl)-ethyl]-8-aza-bicyclo[3.2.1]oct-3-ylamino}-methyl)-4H-pyrido[3,2-b][1,4]thiazin-3-one

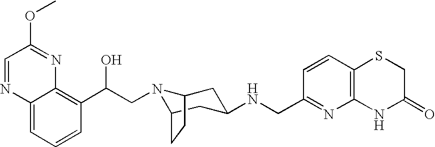

The compound was prepared as in example 1k from aldehyde (17h).

¹H NMR (300 MHz, d₆-DMSO): δ: 10.86 (s, 1H), 8.60 (s, 1H), 7.91-7.88 (m, 2H), 7.72-7.62 (m, 2H), 7.06 (d, 1H), 5.65-5.61 (m, 1H), 4.04 (s, 3H), 3.67 (s, 2H), 3.52 (s, 2H), 3.39-3.27 (m, 4H), 2.87-2.82 (m, 1H), 2.76-2.69 (m, 1H), 1.88-1.66 (m, 4H), 1.50-1.32 (m, 4H)

### Example 80

### 6-({8-[2-Hydroxy-2-(3-methoxy-quinoxalin-5-yl)-ethyl]-8-aza-bicyclo[3.2.1]oct-3-ylamino}-methyl)-4H-benzo[1,4]oxazin-3-one

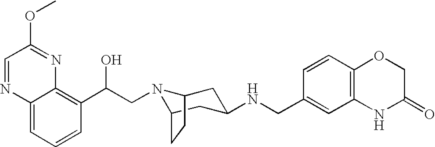

The compound was prepared as in example 1k from aldehyde (1j).

¹H NMR (300 MHz, d₆-DMSO): δ: 10.82 (s, 1H), 8.62 (s, 1H), 7.92-7.89 (m, 2H), 7.68-7.63 (m, 1H), 6.99-6.91 (m, 3H), 5.71-5.67 (m, 1H), 4.56 (s, 2H), 4.06 (s, 3H), 3.82 (s, 2H), 3.58-3.34 (m, 4H), 3.20-3.03 (m, 1H), 2.97-2.83 (m, 1H), 1.96-1.42 (m, 9H)

### Example 81

### 2-{3-[(Benzo[1,3]dioxol-5-ylmethyl)-amino]-8-aza-bicyclo[3.2.1]oct-8-yl}-1-(3-methoxy-quinoxalin-5-yl)-ethanol

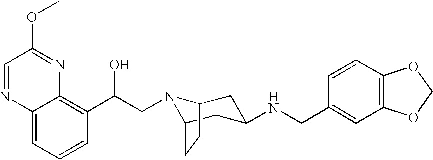

The compound was prepared as in example 1k from benzo[1,3]dioxole-5-carbaldehyde.

¹H NMR (300 MHz, d₆-DMSO): δ: 8.62 (s, 1H), 7.93-7.88 (m, 2H), 7.68-7.63 (m, 1H), 7.05 (s, 1H), 6.93 (s, 2H), 5.71-5.67 (m, 1H), 5.14 (bs, 1H), 4.06 (s, 3H), 3.84 (s, 2H), 3.60-3.35 (m, 3H), 3.20-3.05 (m, 1H), 2.97-2.84 (m, 1H), 1.98-1.42 (m, 9H)

### Example 82

### 2-{3-[(Benzo[1,2,5]thiadiazol-5-ylmethyl)-amino]-8-aza-bicyclo[3.2.1]oct-8-yl}-1-(3-methoxy-quinoxalin-5-yl)-ethanol

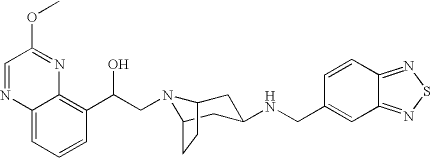

The compound was prepared as in example 1k from benzo[1,2,5]thiadiazole-5-carbaldehyde.

¹H NMR (300 MHz, d₆-DMSO): δ: 8.62 (s, 1H), 8.08-8.03 (m, 2H), 7.95-7.90 (m, 2H), 7.76 (dd, 1H), 7.69-7.64 (m, 1H), 5.78-5.76 (m, 1H), 4.06 (s, 3H), 4.01 (s, 2H), 3.74-3.50 (m, 2H), 3.11-2.95 (m, 2H), 2.74-2.54 (m, 1H), 2.03-1.50 (m, 9H)

### Example 83

### 2-{3-[(Benzo[1,2,5]oxadiazol-5-ylmethyl)-amino]-8-aza-bicyclo[3.2.1]oct-8-yl}-1-(3-methoxy-quinoxalin-5-yl)-ethanol

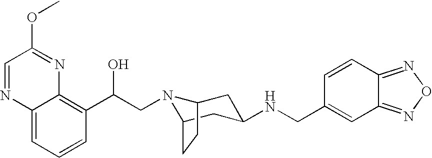

The compound was prepared as in example 1k from benzo[1,2,5]oxadiazole-5-carbaldehyde.

¹H NMR (300 MHz, d₆-DMSO): δ: 8.63 (s, 1H), 8.02-7.91 (m, 4H), 7.71-7.58 (m, 2H), 5.84-5.76 (m, 1H), 4.07 (s, 3H), 3.88 (s, 2H), 3.84-3.58 (m, 2H), 3.16-2.85 (m, 2H), 2.80-2.60 (m, 1H), 2.09-1.55 (m, 9H)

### Example 84

### 7-Fluoro-6-({8-[2-hydroxy-2-(3-methoxy-quinoxalin-5-yl)-ethyl]-8-aza-bicyclo[3.2.1]oct-3-ylamino}-methyl)-4H-benzo[1,4]thiazin-3-one

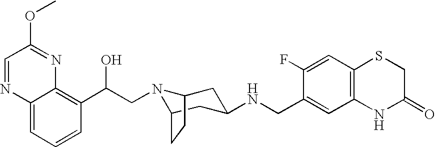

The compound was prepared as in example 1k from aldehyde (24g).

¹H NMR (300 MHz, d₆-DMSO): δ: 10.62 (s, 1H), 8.63 (s, 1H), 7.95-7.91 (m, 2H), 7.70-7.64 (m, 1H), 7.22 (d, 1H), 7.07 (d, 1H), 5.80-5.76 (m, 1H), 4.07 (s, 3H), 3.85-3.75 (m, 3H), 3.51 (s, 2H), 3.15-2.86 (m, 2H), 2.74-2.56 (m, 1H), 2.06-1.50 (m, 9H)

### Example 85

### 6-({8-[2-Hydroxy-2-(3-methoxy-quinoxalin-5-yl)-ethyl]-8-aza-bicyclo[3.2.1]oct-3-ylamino}-methyl)-4H-benzo[1,4]thiazin-3-one

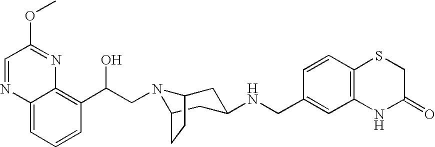

The compound was prepared as in example 1k from aldehyde (6b).

¹H NMR (300 MHz, d₆-DMSO): δ: 10.63 (s, 1H), 8.62 (s, 1H), 7.96-7.90 (m, 2H), 7.68-7.63 (m, 1H), 7.29 (d, 1H), 7.02-6.97 (m, 2H), 5.76-5.69 (m, 1H), 4.06 (s, 3H), 3.78 (s, 2H), 3.61-3.40 (m, 3H), 3.15-2.97 (m, 2H), 2.62-2.49 (m, 1H), 2.00-1.46 (m, 9H)

### Example 86

### 1-(3-Methoxy-quinoxalin-5-yl)-2-[3-((E)-3-phenylallylamino)-8-aza-bicyclo[3.2.1]oct-8-yl]-ethanol

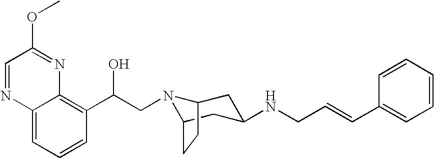

The compound was prepared as in example 1k from cinnamic aldehyde.

¹H NMR (300 MHz, d₆-DMSO): δ: 8.62 (s, 1H), 7.93-7.88 (m, 2H), 7.68-7.62 (m, 1H), 7.45-7.25 (m, 5H), 6.70 (d, 1H), 6.35-6.26 (m, 1H), 5.71-5.66 (m, 1H), 4.06 (s, 3H), 3.63-3.10 (m, 7H), 2.96-2.84 (m, 1H), 1.96-1.46 (m, 9H)

### Example 87

### 6-[({1-[2-Hydroxy-2-(3-methoxy-quinoxalin-5-yl)-ethyl]-piperidin-3-ylmethyl}-amino)-methyl]-4H-benzo[1,4]oxazin-3-one

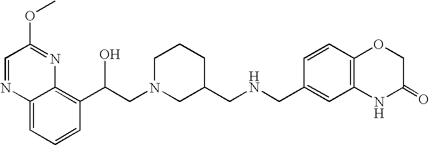

### 87a) {1-[2-Hydroxy-2-(3-methoxy-quinoxalin-5-yl)-ethyl]-piperidin-3-ylmethyl}-carbamic acid tert-butyl ester

To a solution of epoxide (78e) (150 mg) and piperidin-3-ylmethyl-carbamic acid tert-butyl ester (159 mg) in DMF (10 ml) was added lithium perchlorate (95 mg) and stirred under reflux for 3 hours. The mixture was diluted with water (150 ml) water and extracted three times with ethyl acetate. The combined organic layers were washed with brine, dried over magnesium sulfate, filtered and evaporated. The residue was purified by flash chromatography (silica gel, dichloromethane/methanol 9:1) to give the desired product (227 mg).

MS (EI): m/z: 417 [M+H]⁺

### 87b) 2-(3-Aminomethyl-piperidin-1-yl)-1-(3-methoxy-quinoxalin-5-yl)-ethanol

To a solution of Boc-amine (87a) (227 mg) in dichloromethane (10 ml) was added TFA (2 ml) and the mixture stirred for 20 minutes at room temperature. The volatiles were removed and dichloromethane (10 ml) and 2N sodium hydroxide solution (30 ml) added. The aqueous layer was extracted three times with dichloromethane. The combined organic layers were dried over magnesium sulfate, filtered and evaporated to give the desired product (168 mg).

¹H NMR (300 MHz, CDCl₃): δ: 8.41-8.38 (m, 1H), 7.90-7.76 (m, 2H), 7.58-7.50 (m, 1H), 5.78-5.65 (m, 1H), 3.99 (s, 3H), 3.70-3.40 (m, 3H), 3.18-3.00 (m, 1H), 2.95-2.52 (m, 4H), 2.48-2.25 (m, 2H), 2.19-1.99 (m, 1H), 1.97-1.83 (m, 1H), 1.82-1.45 (m, 4H)

### 87c) Title Compound

The compound was prepared as in example 1k from aldehyde (1j).

¹H NMR (300 MHz, d₆-DMSO): δ: 10.74 (s, 1H), 8.60 (s, 1H), 7.89 (d, 2H), 7.67-7.60 (m, 2H), 6.90-6.88 (m, 3H), 5.82-5.79 (m, 1H), 5.11 (bs, 1H), 4.54 (s, 2H), 4.02 (s, 3H), 3.64 (s, 2H), 3.29-3.01 (m, 2H), 3.00-2.80 (m, 1H), 2.78-2.58 (m, 1H), 2.48-2.34 (m, 2H), 2.28-2.10 (m, 1H), 2.04-1.40 (m, 6H), 1.08-0.81 (m, 1H)

### Example 88

### 2-(3-{[(2,3-Dihydro-[1,4]dioxino[2,3-c]pyridin-7-ylmethyl)-amino]-methyl}-piperidin-1-yl)-1-(3-methoxyquinoxalin-5-yl)-ethanol

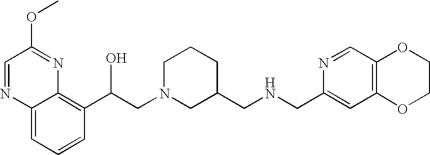

The compound was prepared as in example 1k from aldehyde (30d).

MS (EI): m/z: 466 [M+H]⁺

### Example 89

### 7-Fluoro-6-[({1-[2-hydroxy-2-(3-methoxy-quinoxalin-5-yl)-ethyl]-piperidin-3-ylmethyl}-amino)-methyl]-4H-benzo[1,4]thiazin-3-one

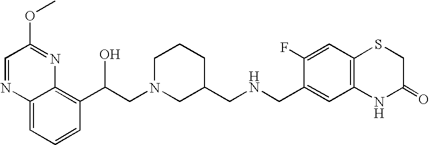

The compound was prepared as in example 1k from aldehyde (24g).

MS (EI): m/z: 512 [M+H]⁺

### Example 90

### 6-[({1-[2-Hydroxy-2-(3-methoxy-quinoxalin-5-yl)-ethyl]-piperidin-3-ylmethyl}-amino)-methyl]-4H-pyrido[3,2-b][1,4]thiazin-3-one

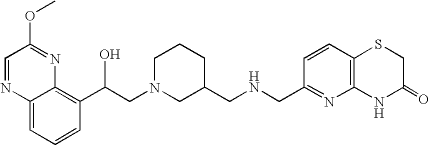

The compound was prepared as in example 1k from aldehyde (17h).

MS (EI): m/z: 495 [M+H]⁺

### Example 91

### 6-[({1-[2-Hydroxy-2-(3-methoxy-quinoxalin-5-yl)-ethyl]-piperidin-3-ylmethyl}-amino)-methyl]-4H-benzo[1,4]thiazin-3-one

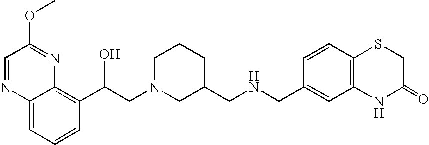

The compound was prepared as in example 1k from aldehyde (6b).

MS (EI): m/z: 494 [M+H]+

### Example 92

### 2-(3-{[(2,3-Dihydro-benzo[1,4]dioxin-6-ylmethyl)-amino]-methyl}-piperidin-1-yl)-1-(3-methoxy-quinoxalin-5-yl)-ethanol

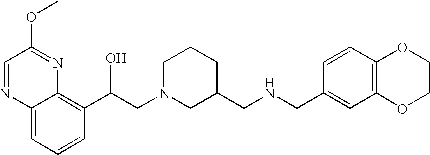

The compound was prepared as in example 1k from 2,3-dihydro-benzo[1,4]dioxine-6-carbaldehyde.

MS (EI): m/z: 465 [M+H]⁺

### Example 93

### 6-[({1-[2-(6-Chloro-3-methoxy-quinoxalin-5-yl)-2-hydroxy-ethyl]-piperidin-3-ylmethyl}-amino)-methyl]-4H-benzo[1,4]oxazin-3-one

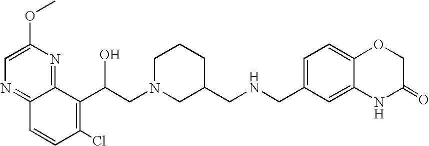

### 93a) 2-Nitro-6-triisopropylsilanyloxy phenyl amine

Chloro triisopropylsilane (62.3 g) was added to a stirred solution of 2-amino-3-nitrophenol (42.9 g) and imidazole (28.4 g) in THF (750 ml) at room temperature. After 18 hours, the resulting mixture was filtered and the filtrate diluted with ethyl acetate (1 l). The organic layer washed with water (2×500 ml), dried over sodium sulfate, filtered and evaporated to give the desired product (91 g) that was used directly for the next step without purification.

¹H NMR (300 MHz, d₆-DMSO): δ: 7.62 (d, 1H), 6.98 (d, 1H), 6.62 (bs, 2H), 6.58-6.54 (m, 1H), 1.41-1.30 (m, 3H), 1.07-1.05 (m, 18H).

MS (EI): m/z: 311 [M+H]⁺

### 93b) 3-Triisopropylsilanyloxy benzene-1,2-diamine

10% palladium on charcoal (8.5 g) was added carefully to a solution of the silyl ether (93a) (91 g) in ethanol (500 ml) and the resulting mixture was hydrogenated for 3 days. The mixture was filtered and the solid washed with ethanol (3×100 ml). The combined ethanol filtrates were evaporated to give the desired product (80.7 g) that was used directly for the next step without purification.

MS (EI): m/z: 281 [M+H]⁺

### 93c) 8-Triisopropylsilanyloxy-1H-quinoxalin-2-one

A 50% solution of ethyl glyoxalate in toluene (60 ml) was added to a solution of diamine (93b) (80.7 g) in ethanol (1 l) at room temperature. The mixture was heated at reflux for 2 hours, cooled to room temperature overnight and then filtered. The solid washed with ice cold ethanol (100 ml) and then dried. The filtrate was evaporated to dryness and acetonitrile was added to the residue. The solid was filtered off, washed with ice-cold acetonitrile (2×100 ml) and combined with the first batch of solid. The combined solids were washed with dichloromethane (2 ml per gram). The desired regioisomer is soluble in dichloromethane whereas the undesired is not. This was performed until all desired regioisomer had dissolved. The dichloromethane washes were evaporated and the residue purified by flash chromatography (silica gel, 0-3% methanol in dichloromethane) to give the desired product (35.6 g).

¹H NMR (300 MHz, CDCl₃): δ: 9.10 (bs, 1H), 8.28 (s, 1H), 7.45 (d, 1H), 7.17-7.13 (m, 1H), 7.00 (d, 1H), 1.44-1.33 (m, 3H), 1.13-1.12 (m, 18H).

MS (EI): m/z: 319 [M+H]⁺

### 93d) 2-Methoxy-8-triisopropylsilanyloxy-quinoxaline

An ice cold stirred solution of quinoxalinone (93c) (48.7 g) in dichloromethane/methanol/acetonitrile (10:1:10, 336 ml) was treated with triethylamine (27.5 ml) followed by a solution of a 2M (trimethylsilyl)diazomethane in hexane (100 ml). The mixture was stirred at room temperature overnight and then evaporated. The crude product was purified by flash chromatography (silica gel, dichloromethane) to give the desired product (26.9 g).

¹H NMR (300 MHz, CDCl₃): δ: 8.45 (s, 1H), 7.62 (d, 1H), 7.41-7.37 (m, 1H), 7.14 (d, 1H), 4.08 (s, 3H) 1.44-1.33 (m, 3H), 1.15-1.13 (m, 18H).

MS (EI): m/z: 333 [M+H]⁺

### 93e) 3-Methoxy-quinoxalin-5-ol

Caesium fluoride (17.98 g) was added to a stirred solution of methoxyquinoxaline (93d) (26.3 g) in THF/methanol (2:1, 750 ml) at room temperature. The mixture was stirred for 30 minutes and then evaporated. The residue was partitioned between diethyl ether (200 ml) and 2N hydrochloric acid (200 ml). The organic layer was separated and the aqueous layer extracted with diethyl ether (3×100 ml). The combined organic layers were dried over magnesium sulfate, filtered and evaporated to give the desired product (15.72 g).

¹H NMR (300 MHz, CDCl₃): δ: 8.49 (s, 1H), 7.56 (d, 1H), 7.48-7.34 (m, 1H), 7.18 (d, 1H), 4.09 (s, 3H).

MS (EI): m/z: 177 [M+H]⁺

### 93f) 6-Chloro-3-methoxy-quinoxalin-5-ol

3-Methoxy-quinoxalin-5-ol (93e) (5 g) was dissolved in acetic acid (200 ml), NCS (4.2 g) was added and the mixture heated to 50° C. over night. Then the mixture was cooled and evaporated. Excess sodium bicarbonate solution was added, the solid collected, washed with water and dried in vacuo at 40° C. over night to give the desired product (5.98 g).

¹H NMR (300 MHz, d₆-DMSO): δ: 10.04 (bs, 1H), 8.67 (s, 1H), 7.59 (d, 1H), 7.11 (d, 1H), 4.11 (s, 3H)

### 93g) Trifluoro-methanesulfonic acid 6-chloro-3-methoxyquinoxalin-5-yl ester

Chloroquinoxalinol (93f) (5.98 g) was suspended in dichloromethane (196 ml), cooled to 0° C., treated with 2,6-lutidine (15 ml), DMAP (520 mg) and trifluoromethane sulfonic acid anhydride (9.5 ml). The mixture was stirred at this temperature for 4 hours and then diluted with saturated ammoniumchloride solution and extracted twice with dichloromethane. The combined organic layers were washed with brine, dried over magnesium sulfate, filtered and evaporated. The residue was purified by flash chromatography (silica gel, ethyl acetate/hexane 2:8) to give the desired product (9.21 g).

¹H NMR (300 MHz, CDCl₃): δ: 8.57 (s, 1H), 7.60 (d, 1H), 7.44 (d, 1H), 4.11 (s, 3H)

### 93h) 7-Chloro-2-methoxy-8-vinyl-quinoxaline

Triflate (93g) (9.21 g) was dissolved in dimethoxyethane (370 ml), tetrakis(triphenylphosphin)palladium (0.93 g) added and the mixture stirred for 20 minutes at room temperature. Then potassium carbonate (3.71 g), water (99 ml) and 2,4,6-trivinyl-cyclotriboroxane pyridin-complex (2.61 g) were added, the mixture stirred at 100° C. for 2 hours and then cooled to room temperature. Water (30 ml) was added and the aqueous layer extracted with ether. The combined organic layers were dried over magnesium sulfate, filtered and evaporated. The residue was purified by flash chromatography (silica gel, ethyl acetate/hexane 1:1, 2:1) to give the desired product (5.62 g).

¹H NMR (300 MHz, CDCl₃): δ: 8.51 (s, 1H), 7.74 (d, 1H), 7.57 (d, 1H), 5.93 (dd, 1H), 5.42 (dd, 1H), 4.07 (s, 3H), 4.06-4.02 (m, 1H)

### 93i) 1-(6-Chloro-3-methoxy-quinoxalin-5-yl)-ethane-1,2-diol (enantiomer 1)

Vinylquinoxaline (93h) (2.8 g) was dissolved in water (94 ml) and tert-butanol (94 ml), treated with AD mix beta (27.2 g) and stirred at 0° C. for 2 days. The mixture was treated with sodium metabisulfite (19.5 g) at 0° C., stirred for 60 minutes at this temperature and then filtered. The filtrate was evaporated, the residue dissolved treated in water and extracted twice with ethyl acetate. The combined organic layers were washed with brine, dried over magnesium sulfate, filtered and evaporated. The residue was purified by flash chromatography (silica gel, ethyl acetate) to give the desired product (1.43 g).

¹H NMR (300 MHz, d₆-DMSO): δ: 8.66 (s, 1H), 7.84-7.79 (m, 2H), 5.60-5.54 (m, 1H), 5.41-5.39 (m, 1H), 4.74-4.70 (m, 1H), 4.08 (s, 3H), 3.78-3.67 (m, 1H), 3.50-3.43 (m, 1H)

### 93j) 7-Chloro-2-methoxy-8-oxiranyl-quinoxaline

A mixture of diol (93i) (1.4 g), triphenyl phosphine (2.16 g) and diethyl azodicarboxylate (1.28 ml) in benzene (20 ml) is refluxed over night. After evaporation of the solvent, the residue was purified by flash chromatography (silica gel, ethyl acetate/hexane 7:3) to give the desired product (796 mg).

¹H NMR (300 MHz, CDCl₃): δ: 8.53 (s, 1H), 7.56 (d, 1H), 7.38 (d, 1H), 4.78-4.76 (m, 1H), 4.06 (s, 3H), 3.25-3.21 (m, 1H), 2.74-2.72 (m, 1H)

### 93k) {1-[2-(6-Chloro-3-methoxy-quinoxalin-5-yl)-2-hydroxy-ethyl]-piperidin-3-ylmethyl}-carbamic acid tert-butyl ester

To a solution of epoxide (93j) (600 mg) and piperidin-3-ylmethyl-carbamic acid tert-butyl ester (652 mg) in DMF (10 ml) was added lithiumperchlorate (324 mg) and the mixture stirred at 170° C. for 3 hours. Water (150 ml) was added to the mixture and the aqueous layer extracted three times with ethyl acetate. The combined organic layers were washed with brine, dried over magnesium sulfate, filtered and evaporated. The residue was purified by flash chromatography (silica gel, dichloromethane/methanol 9:1) to give the desired product (1.10 g).

MS (EI): m/z: 451 [M+H]⁺

### 93i) 2-(3-Aminomethyl-piperidin-1-yl)-1-(6-chloro-3-methoxyquinoxalin-5-yl)-ethanol

Boc-amine (93k) (1.1 g) was dissolved in dichloromethane (20 ml), treated with TFA (2 ml) and stirred for 4 hours at room temperature. The mixture was made alkaline with 2N sodium hydroxide solution and the layers were separated. The aqueous layer was extracted once more with dichloromethane. The combined organic layers were washed with brine, dried over magnesium sulfate, filtered and evaporated. The residue was purified by flash chromatography (silica gel, dichloromethane/methanol 9:1+1% ammonia) to give the desired product (501 mg).

MS (EI): m/z: 351 [M+H]⁺

### 93m) Title Compound

The compound was prepared as in example 1k from aldehyde (1j).

¹H NMR (300 MHz, d₆-DMSO): δ: 10.71 (s, 1H), 8.76 (s, 1H), 7.95-7.81 (m, 2H), 6.98-6.84 (m, 3H), 5.84-5.75 (m, 1H), 5.15 (bs, 1H), 4.57 (s, 2H), 4.10 (s, 3H), 3.65-3.56 (m, 2H), 3.33-3.22 (m, 1H), 3.15-3.04 (m, 1H), 2.99-2.81 (m, 2H), 2.64-2.46 (m, 2H), 2.44-2.26 (m, 2H), 2.19-2.07 (m, 1H), 1.79-1.44 (m, 4H), 1.01-0.84 (m, 1H)

### Example 94

### 2-(3-{[(Benzo[1,3]dioxol-5-ylmethyl)-amino]-methyl}-piperidin-1-yl)-1-(6-chloro-3-methoxy-quinoxalin-5-yl)-ethanol

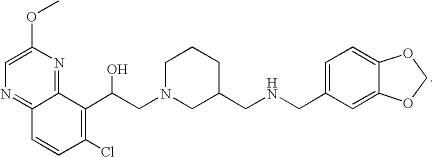

The compound was prepared as in example 1k from benzo[1,3]dioxole-5-carbaldehyde.

MS (EI): m/z: 485 [M+H]⁺

### Example 95

### 1-(6-Chloro-3-methoxy-quinoxalin-5-yl)-2-(3-{[(2,3-dihydro-benzo[1,4]dioxin-6-ylmethyl)-amino]-methyl}-piperidin-1-yl)-ethanol

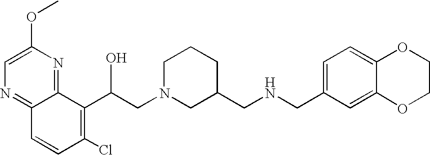

The compound was prepared as in example 1k from 2,3-dihydro-benzo[1,4]dioxine-6-carbaldehyde.

MS (EI): m/z: 499 [M+H]⁺

### Example 96

### 6-[({1-[2-(6-Chloro-3-methoxy-quinoxalin-5-yl)-2-hydroxy-ethyl]-piperidin-3-ylmethyl}-amino)-methyl]-4H-benzo[1,4]thiazin-3-one

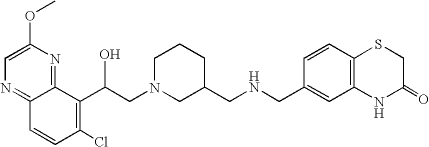

The compound was prepared as in example 1k from aldehyde (6b).

MS (EI: m/z: 528 [M+H]⁺

### Example 97

### 1-(6-Chloro-3-methoxy-quinoxalin-5-yl)-2-(3-{[(2,3-dihydro-[1,4]dioxino[2,3-c]-pyridin-7-ylmethyl)-amino]-methyl}-piperidin-1-yl)-ethanol

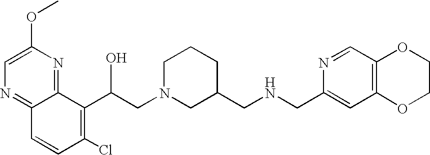

The compound was prepared as in example 1k from aldehyde (30d).

MS (EI): m/z: 500 [M+H]⁺

### Example 98

### 6-[({1-[2-(6-Chloro-3-methoxy-quinoxalin-5-yl)-2-hydroxy-ethyl]-piperidin-3-ylmethyl}-amino)-methyl]-7-fluoro-4H-benzo[1,4]thiazin-3-one

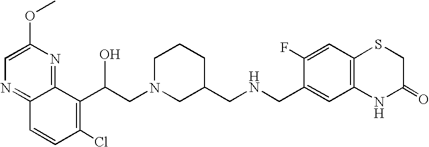

The compound was prepared as in example 1k from aldehyde (24g).

MS (EI): m/z: 546 [M+H]⁺

### Example 99

### 1-(6-Chloro-3-methoxy-quinoxalin-5-yl)-2-{3-[((E)-3-phenyl-allylamino)-methyl]-piperidin-1-yl}-ethanol

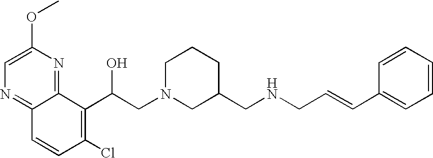

The compound was prepared as in example 1k from cinnamic aldehyde.

MS (EI): m/z: 467 [M+H]⁺

### Example 100

### 6-({8-[2-(6-Chloro-3-methoxy-quinoxalin-5-yl)-2-hydroxy-ethyl]-8-aza-bicyclo[3.2.1]oct-3-ylamino}-methyl)-4H-benzo[1,4]oxazin-3-one

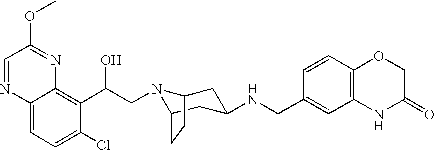

### 100a) {8-[2-(6-Chloro-3-methoxy-quinoxalin-5-yl)-2-hydroxy-ethyl]-8-aza-bicyclo[3.2.1]oct-3-yl}-carbamic acid tert-butyl ester

To a solution of epoxide (93j) (708 mg) and amine (8g) (678 mg) in DMF (20 ml) was added lithium perchlorate (373 mg). The mixture was stirred for 7 days at room temperature. Water (150 ml) was added and the mixture extracted with ethyl acetate. The combined organic layers were dried over magnesium sulfate, filtered and evaporated. The residue was purified by flash chromatography (silica gel, dichloromethane/methanol 19:1) to give the desired product (1.15 g).

MS (EI): m/z: 463 [M+H]⁺

### 100b) 2-(3-Amino-8-aza-bicyclo[3.2.1]oct-8-yl)-1-(6-chloro-3-methoxy-quinoxalin-5-yl)-ethanol

To a solution of Boc-amine (100a) (1.1 g) in dichloromethane (20 ml) was added TFA (10 ml) and the mixture stirred for 2 hours at room temperature. The volatiles were removed and dichloromethane (50 ml) and 2N sodium hydroxide solution (50 ml) added. The aqueous layer was extracted three times with dichloromethane. The combined organic layers were dried over magnesium sulfate, filtered and evaporated. The residue was purified by flash chromatography (silica gel, dichloromethane/methanol 19:1) to give the desired product (634 mg).

MS (EI): m/z: 363 [M+H]⁺

### 100c) Title Compound

The compound was prepared as in example 1k from 2,3-dihydro-benzo[1,4]dioxine-6-carbaldehyde.

MS (EI): m/z: 524 [M+H]⁺

### Example 101

### 6-({8-[2-(6-Chloro-3-methoxy-quinoxalin-5-yl)-2-hydroxy-ethyl]-8-aza-bicyclo[3.2.1]oct-3-ylamino}-methyl)-4H-pyrido[3,2-b][1,4]thiazin-3-one

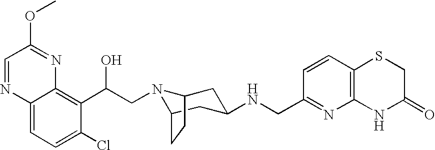

The compound was prepared as in example 1k from aldehyde (17h).

MS (EI): m/z: 541 [M+H]⁺

### Example 102

### 1-(6-Chloro-3-methoxy-quinoxalin-5-yl)-2-(3-[(2,3-dihydro-benzo[1,4]dioxin-6-ylmethyl)-amino]-8-aza-bicyclo[3.2.1]oct-8-yl)-ethanol

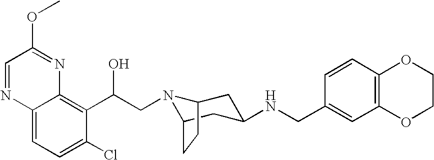

The compound was prepared as in example 1k from 2,3-dihydro-benzo[1,4]dioxine-6-carbaldehyde.

MS (EI): m/z: 511 [M+H]⁺

### Example 103

### 6-({8-[2-(6-Chloro-3-methoxy-quinoxalin-5-yl)-2-hydroxy-ethyl]-8-aza-bicyclo[3.2.1]oct-3-ylamino}-methyl)-4H-benzo[1,4]thiazin-3-one

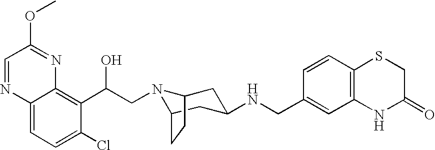

The compound was prepared as in example 1k from aldehyde (6b).

MS (EI): m/z: 540 [M+H]⁺

### Example 104

### 6-({8-[2-(6-Chloro-3-methoxy-quinoxalin-5-yl)-2-hydroxy-ethyl]-8-aza-bicyclo[3.2.1]oct-3-ylamino}-methyl)-7-fluoro-4H-benzo[1,4]thiazin-3-one

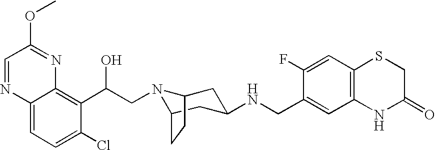

The compound was prepared as in example 1k from aldehyde (24g).

MS (EI): m/z: 558 [M+H]⁺

### Example 105

### 3-{[(3-Oxo-3,4-dihydro-2H-pyrido[3,2-b][1,4]thiazin-6-ylmethyl)-amino]-methyl}-cyclohexanecarboxylic acid (3-methoxy-quinoxalin-5-yl)-amide

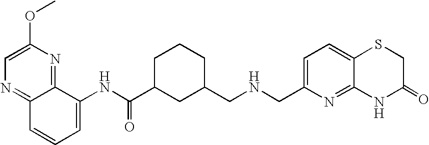

### 105a) 1,1,1-Trifluoromethanesulfonic acid 3-methoxyquinoxalin-5-yl ester

Phenyltrifluoromethanesulfonimide (43.2 g) and triethylamine (16.9 ml) were added to quinoxalinol (93e) (13.24 g) in dry dichloromethane (125 ml) at room temperature and stirred at this temperature for 16 hours. Then saturated sodium carbonate solution (100 ml) was added and the mixture extracted with dichloromethane (5×100 ml). The combined organic extracts were washed with water (4×50 ml), brine (150 ml), dried over sodium sulfate, filtered and evaporated. The crude product was purified by flash chromatography (silica gel, dichloromethane/n-heptane 1:1 to 3:1) to give the desired product (20.2 g).

MS (EI): m/z: 309 [M+H]⁺

### 105b) [3-(3-Methoxy-quinoxalin-5-ylcarbamoyl)-cyclohexylmethyl]-carbamic acid tert-butyl ester

A mixture of amide (46a) (1.5 g), caesium carbonate (2.44 g), tris(dibenzylideneacetone) dipalladium (0) chloroform complex (0.108 g) and 4,5-bis(diphenylphosphino)-9,9-dimethylxanthene (0.208 g) in dry dioxane (50 ml) was sonicated under nitrogen for 10 minutes, during which the solution turned brown. To this solution was added triflate (105a) (1.8 g) and the mixture was heated at 100° C. for 24 hours. After cooling to room temperature, the mixture was centrifuged, the supernatant removed and evaporated. The crude product was purified by flash chromatography (silica gel, ethyl acetate/n-heptane 3:2) to give desired product (1.84 g).

¹H NMR (300 MHz, d₆-DMSO): δ: 9.56 (s, 1H), 8.67 (s, 1H), 8.48 (dd, 1H), 7.72 (dd, 1H), 7.62-7.57 (m, 1H), 7.91-7.83 (m, 1H), 4.17 (s, 3H), 2.94-2.74 (m, 2H), 2.72-2.56 (m, 1H), 2.05-1.89 (m, 2H), 1.87-1.76 (m, 1H), 1.74-1.64 (m, 1H), 1.61-1.45 (m, 1H), 1.37 (s, 9H), 1.23-1.02 (m, 2H), 0.95-0.79 (m, 2H)

### 105c) 3-Aminomethyl-cyclohexanecarboxylic acid (3-methoxy-quinoxalin-5-yl)-amide

Sieves 3A (1.89 g) were suspended in dry dichloromethane (33 ml), cooled with an ice/water bath and treated with a solution of Boc-amine (105b) (1.3 g) in dry dichloromethane (17 ml). Then boron trifluoride etherate (1.97 ml) in dry dichloromethane (17 ml) was added over a period of 45 minutes. The mixture was stirred at room temperature over night. The sieves were filtered off and washed with ethyl acetate, dichloromethane and methanol. The mixture was concentrated and the residue triturated with dichloromethane/methanol 9:1. The precipitate was filtered off and washed with pentane to give the desired product (765 mg).

¹H NMR (300 MHz, d₆-DMSO): δ: 9.59 (s, 1H), 8.68 (s, 1H), 8.48 (dd, 1H), 7.74 (dd, 1H), 7.68 (bs, 2H), 7.63-7.57 (m, 1H), 4.17 (s, 3H), 2.82-2.65 (m, 3H), 2.09-1.96 (m, 2H), 1.92-1.62 (m, 3H), 1.50-1.15 (m, 3H), 1.05-0.89 (m, 1H)

### 105d) Title Compound

The compound was prepared as in example 1k from aldehyde (17h).

¹H NMR (300 MHz, d₆-DMSO): δ: 10.80 (s, 1H), 8.59 (s, 1H), 8.41 (d, 1H), 7.72-7.61 (m, 2H), 7.58-7.46 (m, 1H), 7.03 (d, 1H), 4.05 (s, 3H), 3.65 (s, 2H), 3.45 (s, 2H), 3.20 (s, 2H), 2.70-2.52 (m, 1H), 2.45-2.30 (m, 3H), 1.98-1.86 (m, 1H), 1.84-1.67 (m, 2H), 1.63-1.45 (m, 1H), 1.40-1.00 (m, 3H), 0.94-0.74 (m, 1H)

### Example 106

### 3-{[(7-Fluoro-3-oxo-3,4-dihydro-2H-benzo[1,4]thiazin-6-ylmethyl)-amino]-methyl}-cyclohexanecarboxylic acid (3-methoxy-quinoxalin-5-yl)-amide

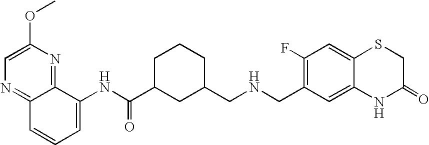

The compound was prepared as in example 1k from aldehyde (24g).

MS (EI): m/z: 510 [M+H]⁺

### Example 107

### 3-{[(2,3-Dihydro-[1,4]dioxino[2,3-c]pyridin-7-ylmethyl)-amino]-methyl}-cyclohexanecarboxylic acid (3-methoxy-quinoxalin-5-yl)-amide

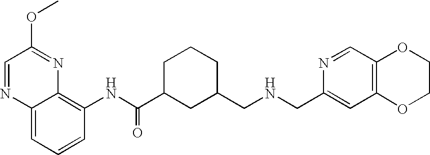

The compound was prepared as in example 1k from aldehyde (30d).

¹H NMR (300 MHz, d₆-DMSO): δ: 9.60 (s, 1H), 8.67 (s, 1H), 8.46 (d, 1H), 8.13 (s, 1H), 7.74 (d, 1H), 7.64-7.55 (m, 1H), 7.08 (s, 1H), 4.41-4.26 (m, 4H), 4.15 (s, 3H), 3.98 (s, 2H), 2.78-2.64 (m, 3H), 2.15-1.94 (m, 2H), 1.88-1.72 (m, 3H), 1.48-1.12 (m, 4H), 1.03-0.84 (m, 1H)

### Example 108

### 3-{[(3-Oxo-3,4-dihydro-2H-pyrido[3,2-b][1,4]thiazin-6-ylmethyl)-amino]-methyl}-cyclohexanecarboxylic acid (2-methoxy-quinolin-8-yl)-amide

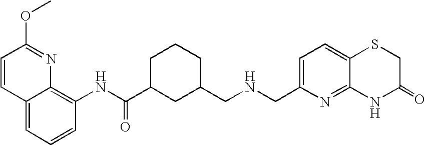

### 108a) 8-Benzyloxyquinoline-2-ol

Benzyl bromide (26.55 g) was added to a stirred solution of 2,8-quinolinediol (25 g) and DBU (30 ml) in 2-propanol (300 ml) at room temperature. The reaction was refluxed for 16 hours, cooled to room temperature and evaporated. The residue was taken up in dichloromethane (250 ml), washed with 0.5M sodium hydroxide solution (2×100 ml), 10% aqueous hydrochloric acid solution (2×100 ml) and water (100 ml), dried over sodium sulfate, filtered and evaporated. The residue was triturated with diethyl ether. The solid was filtered off, washed with diethyl ether and dried to give the desired product (32.3 g).

¹H NMR (300 MHz, d₆-DMSO): δ: 10.82 (s, 1H), 7.87 (d, 1H), 7.58 (d, 2H) 7.39-7.35 (m, 2H), 7.32-7.28 (m, 1H), 7.23-7.19 (m, 2H), 7.09-7.05 (m, 1H), 6.53 (d, 1H), 5.29 (s, 2H).

MS (EI) m/z: 252 [M+H]⁺

### 108b) 8-Benzyloxy-2-chloroquinoline

Quinolinol (108a) (31.6 g) was added to phosphorus oxychloride (225 ml) and the solution was stirred at room temperature for 48 hours. The excess phosphorus oxychloride was evaporated and the residue dissolved in toluene (500 ml). The organic layer was washed with saturated bicarbonate solution (3×150 ml) and water (150 ml), dried over sodium sulfate, filtered and evaporated. The residue was triturated with cyclohexane. The solid was filtered off, washed with cyclohexane and dried to give desired product (29.2 g).

¹H NMR (300 MHz, d₆-DMSO): δ: 8.38 (d, 1H), 7.59-7.51 (m, 5H) 7.43-7.39 (m, 2H), 7.36-7.33 (m, 2H), 5.31 (s, 2H)

MS (EI): m/z: 292 [M+Na]⁺

### 108c) 8-Benzyloxy-2-methoxyquinoline

A solution of chloroquinoline (108b) (28.3 g) in dry toluene (40 ml) was added dropwise to a stirred 25 wt % solution of sodium methoxide (300 ml) at room temperature. The resulting solution was heated at 70° C. for 14 hours, cooled, quenched with ice (300 g) and extracted with toluene (4×150 ml). The combined organic extracts were dried over sodium sulfate, filtered and evaporated to give the desired product (27 g), which was used directly for the next reaction without purification.

MS (EI): m/z: 266 [M+H]⁺

### 108d) 8-Hydroxy-2-methoxyquinoline

8-Benzyloxy-2-methoxyquinoline (108c) (26.8 g) was dissolved in ethanol (300 ml), treated with 10% palladium on charcoal (1.5 g) and hydrogenated under an H₂ atmosphere (20 psi) for 5 hours. The reaction mixture was filtered through Celite®, the solid washed with ethanol and the filtrate evaporated to give the desired product (16.5 g).

MS (EI): m/z: 176 [M+H]⁺

### 108e) 1,1,1-Trifluoromethanesulfonic acid 2-methoxyquinolin-8-yl ester

Phenyltrifluoromethanesulfonimide (45.4 g) and triethylamine (17.6 ml) were added to hydroxyquinoline (108d) (14.5 g) in dry DCM (125 ml) at room temperature and heated at 40° C. for 14 hours. After cooling to room temperature an aqueous potassium carbonate solution (250 ml) was added and the mixture was extracted with dichloromethane (5×250 ml). The combined organic extracts were washed with water (4×150 ml) and brine (150 ml), dried over sodium sulfate, filtered and evaporated. The crude product was purified by flash chromatography (silica gel, dichloromethane/n-heptane 1:1, dichloromethane) to give (23.5 g) of the desired product as a white solid.

MS (EI): m/z: 308 [M+H]⁺

### 108f) [3-(2-Methoxy-quinolin-8-ylcarbamoyl)-cyclohexylmethyl]-carbamic acid tert-butyl ester

A mixture of amide (46a) (1.19 g), caesium carbonate (1.82 g), tris(dibenzylideneacetone) dipalladium (0) chloroform complex (0.081 g) and 4,5-bis(diphenylphosphino)-9,9-dimethylxanthene (0.155 g) in dry dioxane (25 ml) was sonicated under nitrogen for 10 minutes, during which the solution turned brown. To this solution was added triflate (108e) (1.15 g) and the mixture was heated at 100° C. for 24 hours. After cooling to room temperature, the mixture was centrifuged, the supernatant removed and evaporated. The product was purified by flash chromatography (silica gel, ethyl acetate/n-heptane 1:2 to 1:1) to give the desired product (0.791 g).

¹H NMR (300 MHz, d₆-DMSO): δ: 9.61 (s, 1H), 8.48 (dd, 1H), 8.27 (d, 1H), 7.58 (dd, 1H), 7.42-7.36 (m, 1H), 7.10 (d, 1H), 6.89-6.84 (m, 1H), 4.11 (s, 3H), 2.94-2.72 (m, 2H), 2.65-2.51 (m, 1H), 2.10-1.95 (m, 2H), 1.86-1.64 (m, 2H), 1.59-1.39 (m, 2H), 1.37 (s, 9H), 1.10-1.03 (m, 2H), 0.95-0.78 (m, 1H)

### 108g) 3-Aminomethyl-cyclohexanecarboxylic acid (2-methoxy-quinolin-8-yl)-amide

Sieves 3A (1.14 g) were suspended in dry dichloromethane (20 ml), cooled with an ice/water bath and treated with a solution of Boc-amine (108f) (780 mg) in dry dichloromethane (10 ml). Then boron trifluoride etherate (1.22 ml) in dry dichloromethane (10 ml) was added over a period of 45 minutes. The mixture was stirred at room temperature over night. The sieves were filtered off and washed with ethyl acetate, dichloromethane and methanol. The mixture was concentrated and the residue triturated with dichloromethane/methanol 9:1. The precipitate was filtered off and washed with pentane to give the desired product (589 mg).

¹H NMR (300 MHz, d₆-DMSO): δ: 9.91 (s, 1H), 8.48 (dd, 1H), 8.28 (d, 1H), 7.68 (bs, 2H), 7.60 (dd, 1H), 7.44-7.26 (m, 1H), 7.10 (d, 1H), 4.11 (s, 3H), 2.82-2.58 (m, 3H), 2.15-2.00 (m, 2H), 1.95-1.58 (m, 3H), 1.52-1.15 (m, 3H), 1.06-0.86 (m, 1H)

### 108h) Title Compound

The compound was prepared as in example 1k from aldehyde (17h).

¹H NMR (300 MHz, d₆-DMSO): δ: 10.86 (s, 1H), 9.63 (s, 1H), 8.49 (dd, 1H), 8.27 (d, 1H), 7.79-7.72 (m, 1H), 7.58 (dd, 1H), 7.42-7.36 (m, 1H), 7.11-7.07 (m, 2H), 4.46 (d, 1H), 4.07 (s, 3H), 3.70 (s, 2H), 3.53 (s, 3H), 2.50-2.35 (m, 2H), 2.25-2.12 (m, 1H), 2.09-1.98 (m, 1H), 1.89-1.73 (m, 1H), 1.68-1.51 (m, 2H), 1.49-1.32 (m, 1H), 1.27-1.18 (m, 2H), 1.01-0.84 (m, 1H)

### Example 109

### 3-{[(Benzo[1,3]dioxol-5-ylmethyl)-amino]-methyl}-cyclohexanecarboxylic acid (2-methoxy-quinolin-8-yl)-amide

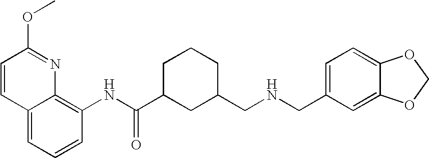

The compound was prepared as in example 1k from benzo[1,3]dioxole-5-carbaldehyde.

¹H NMR (300 MHz, d₆-DMSO): δ: 9.44 (s, 1H), 8.30 (d, 1H), 8.09 (d, 1H), 7.40 (d, 1H), 7.28-7.16 (m, 1H), 6.92 (d, 1H), 6.71-6.55 (m, 3H), 5.80 (s, 2H), 4.21 (d, 1H), 3.89 (s, 3H), 3.45 (s, 2H), 2.29-2.15 (m, 3H), 2.05-1.96 (m, 1H), 1.94-1.74 (m, 1H), 1.70-1.53 (m, 1H), 1.50-1.31 (m, 2H), 1.29-0.95 (m, 3H), 0.90-0.59 (m, 1H)

### Example 110

### 3-{[(2,3-Dihydro-benzo[1,4]dioxin-6-ylmethyl)-amino]-methyl}-cyclohexanecarboxylic acid (2-methoxy-quinolin-8-yl)-amide

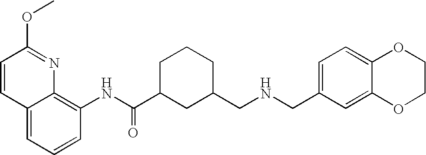

The compound was prepared as in example 1k from 2,3-dihydro-benzo[1,4]dioxine-6-carbaldehyde.

MS (EI): m/z: 462 [M+H]⁺

### Example 111

### 3-{[(2,3-Dihydro-[1,4]dioxino[2,3-c]pyridin-7-ylmethyl)-amino]-methyl}-cyclohexanecarboxylic acid (2-methoxy-quinolin-8-yl)-amide

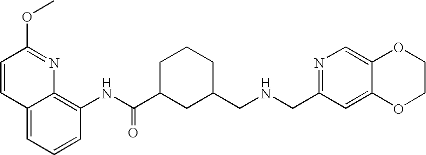

The compound was prepared as in example 1k from aldehyde (30d).

¹H NMR (300 MHz, d₆-DMSO): δ: 9.43 (s, 1H), 8.30 (dd, 1H), 8.07 (d, 1H), 7.87 (s, 1H), 7.39 (dd, 1H), 7.25-7.16 (m, 1H), 6.93 (d, 1H), 6.80 (s, 1H), 4.19-4.05 (m, 4H), 3.86 (s, 3H), 3.60 (s, 2H), 2.47-2.22 (m, 3H), 2.04-1.91 (m, 1H), 1.89-1.75 (m, 1H), 1.68-1.38 (m, 3H), 1.30-0.88 (m, 4H), 0.84-0.63 (m, 1H)

### Example 112

### 3-{[(3-oxo-3,4-dihydro-2H-benzo[1,4]thiazin-6-ylmethyl)-amino]-methyl}-cyclohexanecarboxylic acid (2-methoxy-quinolin-8-yl)-amide

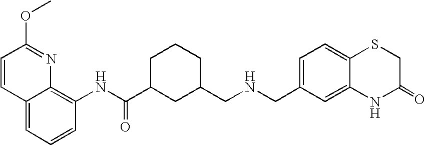

The compound was prepared as in example 1k from aldehyde (6b).

¹H NMR (300 MHz, d₆-DMSO): δ: 10.54 (s, 1H), 9.63 (s, 1H), 8.50 (dd, 1H), 8.28 (d, 1H), 7.58 (dd, 1H), 7.44-7.35 (m, 1H), 7.25 (d, 1H), 7.10 (d, 1H), 7.01-6.90 (m, 2H), 4.06 (s, 3H), 3.65 (s, 2H), 3.44 (s, 2H), 2.67-2.34 (m, 3H), 2.24-2.10 (m, 1H), 2.08-1.95 (m, 1H), 1.90-1.75 (m, 2H), 1.69-1.51 (m, 1H), 1.49-1.28 (m, 2H), 1.26-1.07 (m, 2H), 0.99-0.80 (m, 1H)

The following examples were prepared according to the above described procedures using the appropriate starting materials. The aldehyde used e.g. in example 124 was synthesized as described in WO04058144.

### Example 113

### 1-(6-Chloro-3-methoxy-quinoxalin-5-yl)-2-{3-[(2,3-dihydro-[1,4]dioxino[2,3-c]pyridin-7-ylmethyl)-amino]-8-aza-bicyclo[3.2.1]oct-8-yl}-ethanol

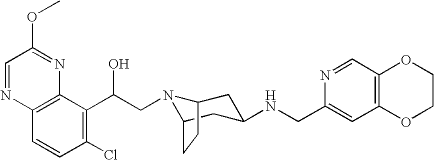

### Example 114

### 2-{3-[(2,3-Dihydro-[1,4]dioxino[2,3-c]pyridin-7-ylmethyl)-amino]-8-aza-bicyclo[3.2.1]oct-8-yl}-1-(2-methoxy-quinolin-8-yl)-ethanol

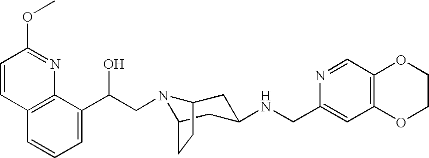

### Example 115

### 6-({8-[2-Hydroxy-2-(2-methoxy-quinolin-8-yl)-ethyl]-8-aza-bicyclo[3.2.1]oct-3-ylamino}-methyl)-4H-pyrido[3,2-b][1,4]thiazin-3-one

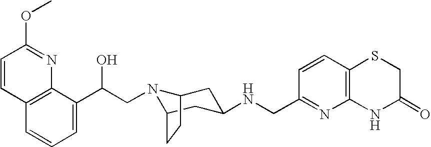

### Example 116

### 6-({8-[2-Hydroxy-2-(2-methoxy-quinolin-8-yl)-ethyl]-8-aza-bicyclo[3.2.1]oct-3-ylamino}-methyl)-4H-benzo[1,4]thiazin-3-one

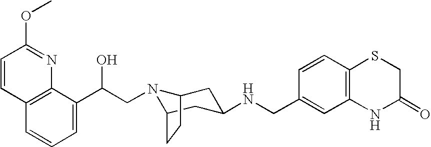

### Example 117

### 6-({8-[2-Hydroxy-2-(2-methoxy-quinolin-8-yl)-ethyl]-8-aza-bicyclo[3.2.1]oct-3-ylamino}-methyl)-4H-benzo[1,4]oxazin-3-one

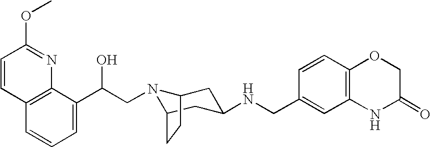

### Example 118

### 2-{3-[(2,3-Dihydro-benzo[1,4]dioxin-6-ylmethyl)-amino]-8-aza-bicyclo[3.2.1]oct-8-yl}-1-(2-methoxy-quinolin-8-yl)-ethanol

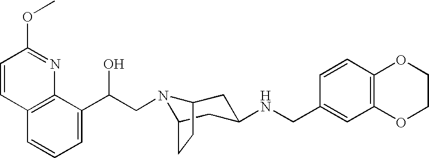

### Example 119

### 6-[({1-[2-Hydroxy-2-(2-methoxy-quinolin-8-yl)-ethyl]-piperidin-3-ylmethyl}-amino)-methyl]-4H-benzo[1,4]thiazin-3-one

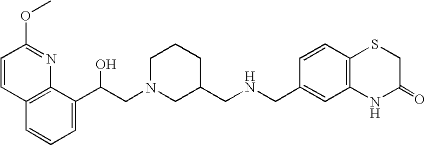

### Example 120

### 6-[({1-[2-Hydroxy-2-(2-methoxy-quinolin-8-yl)-ethyl]-piperidin-3-ylmethyl}-amino)-methyl]-4H-pyrido[3,2-b][1,4]thiazin-3-one

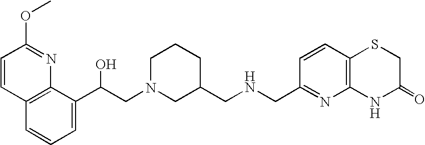

### Example 121

### 6-[({1-[2-Hydroxy-2-(2-methoxy-quinolin-8-yl)-ethyl]-piperidin-3-ylmethyl}-amino)-methyl]-4H-benzo[1,4]oxazin-3-one

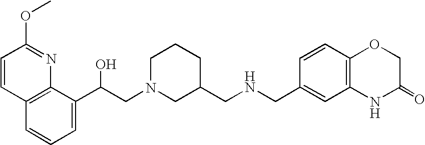

### Example 122

### 2-(3-{[(2,3-Dihydro-[1,4]dioxino[2,3-c]pyridin-7-ylmethyl)-amino]-methyl}-piperidin-1-yl)-1-(2-methoxy-quinolin-8-yl)-ethanol

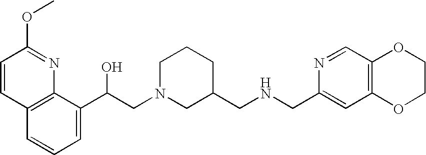

### Examples 123

### 2-(3-{[(2,3-Dihydro-benzo[1,4]dioxin-6-ylmethyl)-amino]-methyl}-piperidin-1-yl)-1-(2-methoxy-quinolin-8-yl)-ethanol

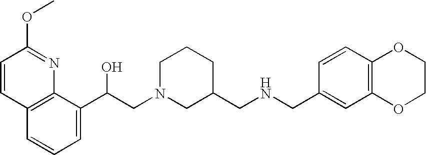

### Example 124

### 6-({8-[2-(6-Chloro-3-methoxy-quinoxalin-5-yl)-2-hydroxy-ethyl]-8-aza-bicyclo[3.2.1]oct-3-ylamino}-methyl)-4H-pyrido[3,2-b][1,4]oxazin-3-one

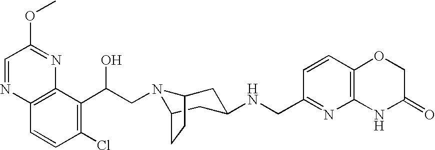

### Example 125

### 6-({8-[2-Hydroxy-2-(3-methoxy-quinolin-5-yl)-ethyl]-8-aza-bicyclo[3.2.1]oct-3-ylamino}-methyl)-4H-pyrido[3,2-b][1,4]oxazin-3-one

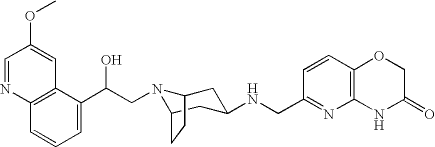

### Example 126

### 6-({8-[2-Hydroxy-2-(3-methoxy-quinoxalin-5-yl)-ethyl]-8-aza-bicyclo[3.2.1]oct-3-ylamino}-methyl)-4H-pyrido[3,2-b][1,4]oxazin-3-one

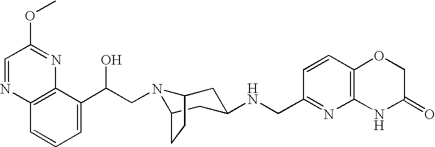

### Example 127

### 6-({8-[2-Hydroxy-2-(2-methoxy-quinolin-8-yl)-ethyl]-8-aza-bicyclo[3.2.1]oct-3-ylamino}-methyl)-4H-pyrido[3,2-b][1,4]oxazin-3-one

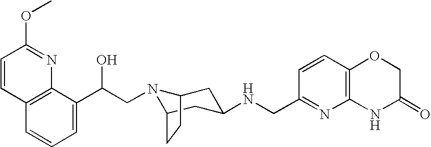

### Example 128

### 6-[({1-[2-Hydroxy-2-(2-methoxy-quinolin-8-yl)-ethyl]-piperidin-3-ylmethyl}-amino)-methyl]-4H-pyrido[3,2-b][1,4]oxazin-3-one

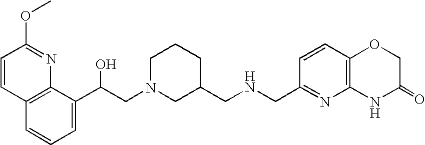

### Example 129

### 6-[({1-[2-Hydroxy-2-(3-methoxy-quinoxalin-5-yl)-ethyl]-piperidin-3-ylmethyl}-amino)-methyl]-4H-pyrido[3,2-b][1,4]oxazin-3-one

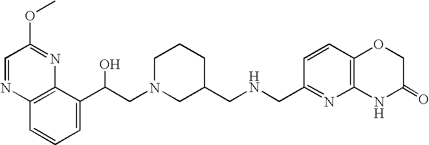

### Example 130

### 6-[({1-[2-(6-Chloro-3-methoxy-quinoxalin-5-yl)-2-hydroxy-ethyl]-piperidin-3-ylmethyl}-amino)-methyl]-4H-pyrido[3,2-b][1,4]oxazin-3-one

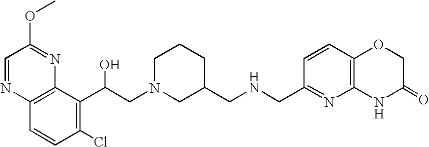

### Example 131

### 6-[({1-[2-Hydroxy-2-(3-methoxy-quinolin-5-yl)-ethyl]-piperidin-3-ylmethyl}-amino)-methyl]-4H-pyrido[3,2-b][1,4]oxazin-3-one

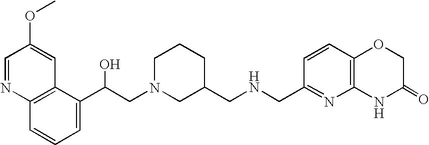

### Example 132

### 6-[({4-[2-Hydroxy-2-(2-methoxy-quinolin-8-yl)-ethyl]-morpholin-2-ylmethyl}-amino)-methyl]-4H-pyrido[3,2-b][1,4]oxazin-3-one

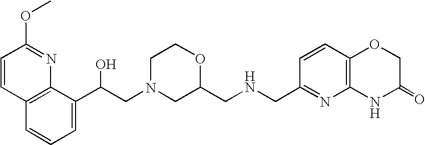

### Example 133

### 6-[({4-[2-Hydroxy-2-(3-methoxy-quinoxalin-5-yl)-ethyl]-morpholin-2-ylmethyl}-amino)-methyl]-4H-pyrido[3,2-b][1,4]oxazin-3-one

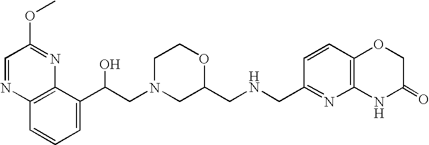

### Example 134

### 6-[({4-[2-(6-Chloro-3-methoxy-quinoxalin-5-yl)-2-hydroxy-ethyl]-morpholin-2-ylmethyl}-amino)-methyl]-4H-pyrido[3,2-b][1,4]oxazin-3-one

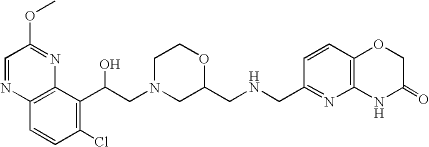

### Example 135

### 6-[({4-[2-Hydroxy-2-(3-methoxy-quinolin-5-yl)-ethyl]-morpholin-2-ylmethyl}-amino)-methyl]-4H-pyrido[3,2-b][1,4]oxazin-3-one

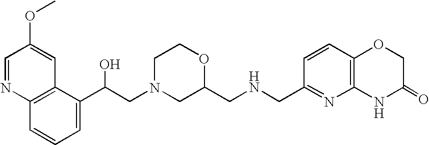

### Example 136

### 6-({8-[2-(3-Chloro-6-methoxy-[1,5]naphthyridin-4-yl)-2-hydroxy-ethyl]-8-aza-bicyclo[3.2.1]oct-3-ylamino}-methyl)-4H-pyrido[3,2-b][1,4]oxazin-3-one

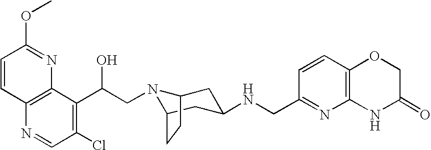

### Example 137

### 6-({8-[2-Hydroxy-2-(6-methoxy-[1,5]naphthyridin-4-yl)-ethyl]-8-aza-bicyclo[3.2.1]oct-3-ylamino}-methyl)-4H-pyrido[3,2-b][1,4]oxazin-3-one

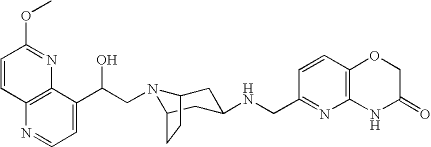

### Example 138

### 3-{[(3-Oxo-3,4-dihydro-2H-pyrido[3,2-b][1,4]oxazin-6-ylmethyl)-amino]-methyl}-cyclohexanecarboxylic acid (3-chloro-6-methoxy-[1,5]naphthyridin-4-yl)-amide

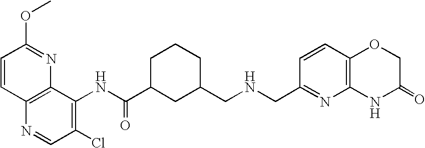

### Example 139

### 3-{[(3-Oxo-3,4-dihydro-2H-pyrido[3,2-b][1,4]oxazin-6-ylmethyl)-amino]-methyl}-cyclohexanecarboxylic acid (6-methoxy-[1,5]naphthyridin-4-yl)-amide

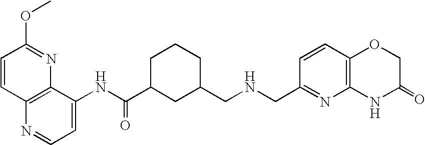

### Example 140

### 6-({8-[2-(7-Chloro-2-methoxy-quinolin-8-yl)-2-hydroxy-ethyl]-8-aza-bicyclo[3.2.1]oct-3-ylamino}-methyl)-4H-pyrido[3,2-b][1,4]oxazin-3-one

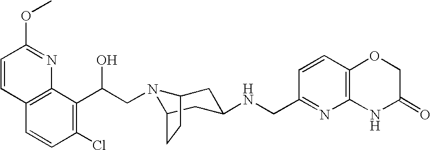

### Example 141

### 6-({8-[2-(7-Chloro-2-methoxy-quinolin-8-yl)-2-hydroxy-ethyl]-8-aza-bicyclo[3.2.1]oct-3-ylamino}-methyl)-4H-benzo[1,4]oxazin-3-one

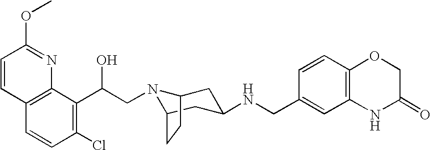

### Example 142

### 6-({8-[2-(7-Chloro-2-methoxy-quinolin-8-yl)-2-hydroxy-ethyl]-8-aza-bicyclo[3.2.1]oct-3-ylamino}-methyl)-4H-pyrido[3,2-b][1,4]thiazin-3-one

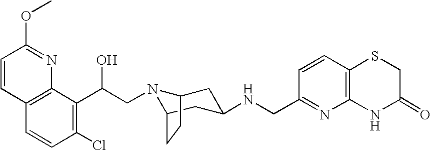

### Example 143

### 6-({8-[2-(7-Chloro-2-methoxy-quinolin-8-yl)-2-hydroxy-ethyl]-8-aza-bicyclo[3.2.1]oct-3-ylamino}-methyl)-4H-benzo[1,4]thiazin-3-one

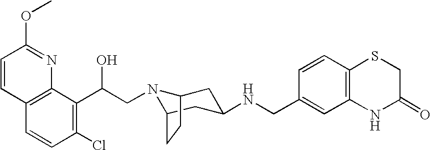

### Example 144

### 6-[({1-[2-(7-Chloro-2-methoxy-quinolin-8-yl)-2-hydroxy-ethyl]-piperidin-3-ylmethyl}-amino)-methyl]-4H-pyrido[3,2-b][1,4]oxazin-3-one

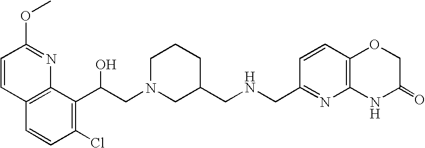

### Example 145

### 6-[({1-[2-(7-Chloro-2-methoxy-quinolin-8-yl)-2-hydroxy-ethyl]-piperidin-3-ylmethyl}-amino)-methyl]-4H-benzo[1,4]oxazin-3-one

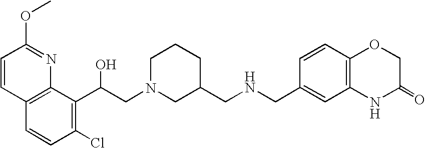

### Example 146

### 6-[({1-[2-(7-Chloro-2-methoxy-quinolin-8-yl)-2-hydroxy-ethyl]-piperidin-3-ylmethyl}-amino)-methyl]-4H-pyrido[3,2-b][1,4]thiazin-3-one

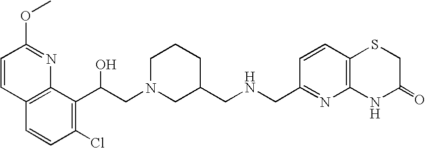

### Example 147

### 6-[({1-[2-(7-Chloro-2-methoxy-quinolin-8-yl)-2-hydroxy-ethyl]-piperidin-3-ylmethyl}-amino)-methyl]-4H-benzo[1,4]thiazin-3-one

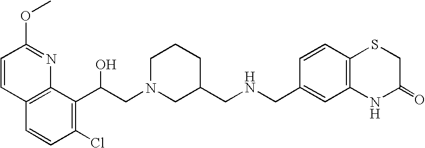

The MIC (μg/ml) of the examples against various organisms was determined: A. baumannii ATCC19606, E. cloacae ATCC23355, E. coli ATCC25922, K. pneumoniae ATCC27736, P. mirabilis ATCC29906, P. aeruginosa ATCC27853, S. maltophilia ATCC13637, S. aureus ATCC43300, S. epidermidis ATCC14990, S. haemolyticus ATCC29970, E. faecalis ATCC29212 and E. faecium ATC19434.

Examples 3, 6, 7-11, 13-17, 19, 20, 22, 24-45, 53, 57-63, 65-82, 84-99 105, 107, 108, 110-112 have an MIC of less than or equal to 2 μg/ml against at least two of the above organisms.

1-11. (canceled)

12. A compound of formula (I):
Q-A-R³ (I)
wherein
Q is a group having the following structure: 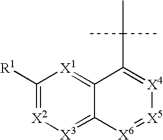
R¹ is a hydrogen atom, a halogen atom, a hydroxy, an amino, a mercapto, an alkyl, a heteroalkyl, an alkyloxy, a heteroalkyloxy, a cycloalkyl, a heterocycloalkyl, an alkylcycloalkyl, a heteroalkylcycloalkyl, a cycloalkyloxy, an alkylcycloalkyloxy, a heterocycloalkyloxy or a heteroalkylcycloalkyloxy group,
X¹, X², X³, X⁴, X⁵ and X⁶ are each independently of the others nitrogen atoms or groups of formula CR²,
R² is a hydrogen atom, a halogen atom, or a hydroxy, amino, alkyl, alkenyl, alkynyl or heteroalkyl group,
R³ is selected from the following groups: 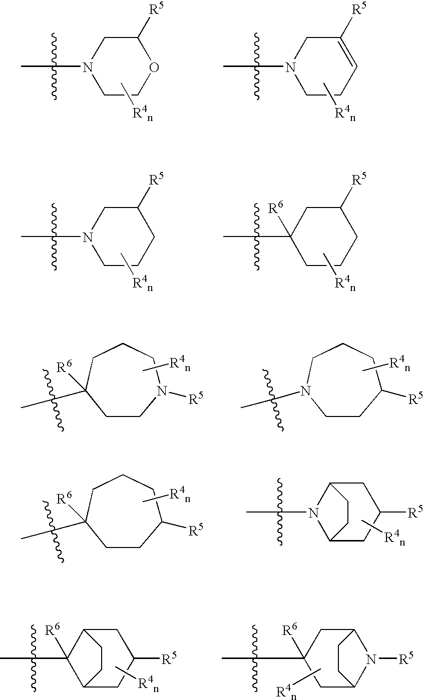
the radicals R⁴, each independently of any other(s), are a halogen atom, a hydroxy, an amino, a nitro or a mercapto group, an alkyl, an alkenyl, an alkynyl, a heteroalkyl, an aryl, a heteroaryl, a cycloalkyl, an alkylcycloalkyl, a heteroalkylcycloalkyl, a heterocycloalkyl, an aralkyl or a heteroaralkyl radical, or two of the radicals R⁴ together form part of an aryl, heteroaryl, cycloalkyl, heterocycloalkyl, alkylcycloalkyl, heteroalkylcycloalkyl, aralkyl or a heteroaralkyl ring system,
R⁵ is an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radical,
R⁶ is a hydrogen atom or R⁷,
R⁷ is a halogen atom, or a hydroxy, alkyl, alkenyl, alkynyl or heteroalkyl group,
n is 0, 1 or 2,
A is selected from the following groups: —NR⁸CO—, —CR⁹R¹⁰CO—, —CR⁹R¹⁰SO₂—, —NR⁸SO₂—, —CR⁹R¹⁰CR¹¹(R¹²)—, —CONR⁸—, —CR⁹R¹⁰NR⁸—, —CR⁹R¹⁰O—, —CR⁹R¹⁰S—, —CR¹¹(OR²)CR¹³R¹⁴, —COR¹³R¹⁴— and —CR⁹R¹⁰CR¹³R¹⁴—,
R⁸ is a hydrogen atom, a trifluoromethyl, a (C₁₋₆)alkyl, a (C₂₋₆)alkenyl, a (C₁₋₆)alkoxycarbonyl, a (C₁₋₆)alkylcarbonyl or an aminocarbonyl group wherein the amino group, if applicable, may be substituted by a (C₁₋₆)alkoxycarbonyl, a (C₁₋₆)alkylcarbonyl, a (C₂₋₆)alkenyloxycarbonyl, a (C₂₋₆)alkenylcarbonyl, a (C₁₋₆)alkyl, a (C₂₋₆)alkenyl and, if applicable, substituted further on by a (C₁₋₆)alkyl or a (C₂₋₆)alkenyl group,
the radicals R⁹, R¹⁰, R¹¹, R¹³ and R¹⁴ are each independently of the others a hydrogen atom, a halogen atom, an azide, a trifluoromethyl, a hydroxy, an amino, a (C₁₋₆)alkyloxy, a (C₁₋₆)alkylthio, a (C₁₋₆)alkyl, a (C₂₋₆)alkenyl, a (C₁₋₆)alkoxycarbonyl, a (C₂₋₆)alkenyloxycarbonyl, a (C₁₋₆)alkylsulphonyl, a (C₂₋₆)alkenylsulphonyl or a (C₁₋₆)aminosulphonyl group wherein the amino group may be, if applicable, substituted by a (C₁₋₆)alkyl or a phenyl group,
R¹² is a hydrogen atom, a trifluoromethyl, a (C₁₋₆)alkyl, a (C₂₋₆)alkenyl, a (C₁₋₆)alkoxycarbonyl, a (C₁₋₆)alkylcarbonyl or an aminocarbonyl group wherein the amino group may be, if applicable, substituted by a (C₁₋₆)alkoxycarbonyl, a (C₁₋₆)alkylcarbonyl, a (C₂₋₆)alkenyloxycarbonyl, a (C₂₋₆)alkenylcarbonyl, a (C₁₋₆)alkyl, a (C₂₋₆)alkenyl group and, if applicable, substituted further on by a (C₁₋₆)alkyl or a (C₂₋₆)alkenyl group,
or a pharmacologically acceptable salt, solvate, hydrate or a pharmacologically acceptable formulation thereof.

13. A compound of claim 12, wherein A is selected from the following groups: —NHCO—, —CH₂CO—, —CH₂SO₂—, —NHSO₂—, —CH₂CH(OH)—, —CH(OH)CH₂—, —CH₂CH₂—, —CONH—, —CH₂N(C₁-C₄alkyl)-, —CH₂O— or —CH₂S—.

14. A compound of claim 12, wherein Q is selected from the following groups:

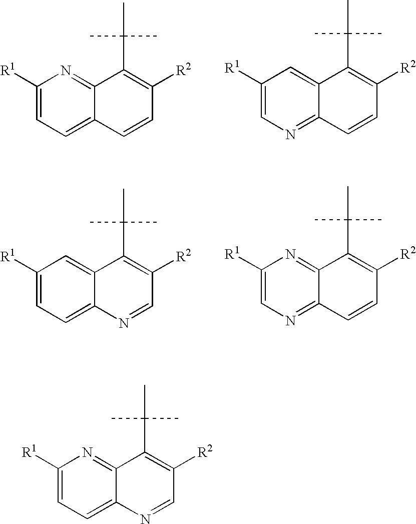

15. A compound of claim 12, wherein R¹ is a methoxy group.

16. A compound of claim 12, wherein R² is a hydrogen atom or a halogen atom.

17. A compound of claim 12, wherein R⁵ is a group of formula —B—Y, B being an alkylene, an alkenylene, an alkynylene, a —NH— or a heteroalkylene group and Y being an aryl, a heteroaryl, an aralkyl, a heteroaralkyl, a cycloalkyl, a heterocycloalkyl, an alkylcycloalkyl or a heteroalkylcycloalkyl group.

18. A compound of claim 17, wherein B is a group of formula —CH₂CH(OH)—, —CH₂NHCH₂—, —NHCH₂CH₂—, —NH—, —CH₂NHCH₂CH₂—, —CH₂CO— or —NHCH₂—.

19. A compound of claim 17, wherein Y has one of the following structures:

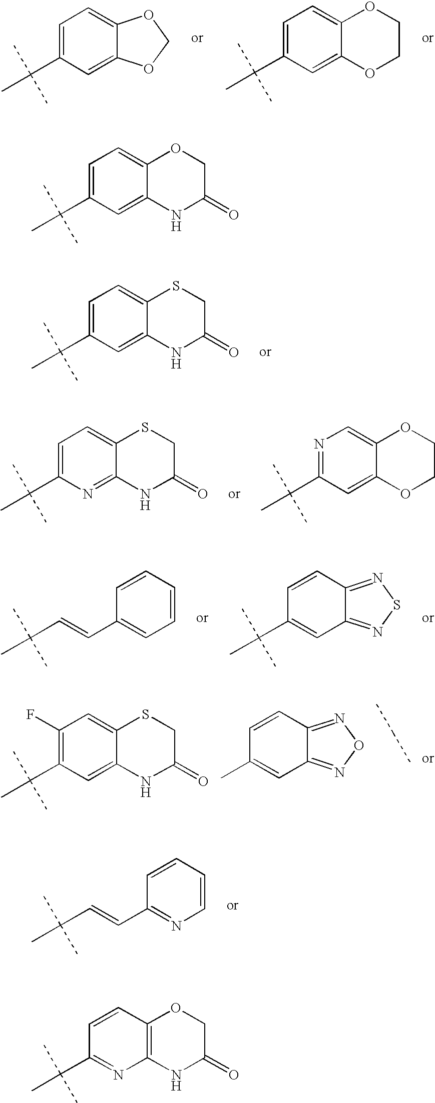

20. A compound of claim 12, wherein R³ is selected from the following groups:

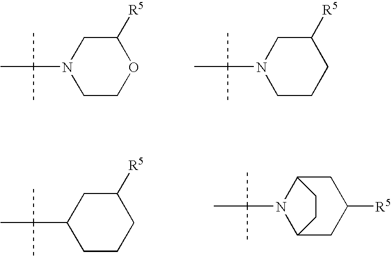

21. A pharmaceutical composition comprising a compound of claim 12.

22. A pharmaceutical composition of claim 12 further comprising one or more carrier substances and/or adjuvants.

23. A method for treating a patient suffering from or susceptible to a bacterial infection, comprising administering to the subject a compound of claim 12.

24. The method of claim 23 wherein the patient is suffering from a bacterial infection.

25. The method of claim 23 wherein the patient is identified as suffering from a bacterial infection and selected for administration of the compound and the compound is administered to the selected patient.
